(19) United States
(12) Patent Application Publication
(10) Pub. No.: US 2007/0111933 A1
(43) Pub. Date: May 17, 2007

(54) Diagnosis and treatment methods related to aging, especially of liver

(76) Inventors: John J. Kopchick, Athens, OH (US); Bruce Kelder, Athens, OH (US); Keith Boyce, Wexford, PA (US); Andres Kriete, Pittsburgh, PA (US)

Correspondence Address: BROWDY AND NEIMARK, P.L.L.C.;624 NINTH STREET, NW, SUITE 300, WASHINGTON, DC, 20001-5303, US

(21) Appl. No.: 10/558,877

(22) PCT Filed: Jun. 2, 2004
(86) PCT No.: PCT/US04/17322

§ 371 Date: Sep. 26, 2006

Related U.S. Application Data
(60) Provisional application No. 60/474,606, filed on Jun. 2, 2003.

(51) Int. Cl. A61K 38/17; A61K 48/00

(52) U.S. Cl. 514/12; 514/44

(57) ABSTRACT

Mouse genes differentially expressed in comparisons of older and younger livers by gene chip analysis have been identified, as have corresponding human genes and proteins. The human molecules, or antagonists thereof, may be used for protection against faster-than-normal biological aging, or to achieve slower-than-normal biological aging. The human molecules may also be used as markers of biological aging.

This application claims the benefit, under 35 USC 119(e), of U.S. Provisional application 60/474,606, filed Jun. 2, 2003, which is hereby incorporated by reference in its entirety.

### CROSS-REFERENCE TO RELATED APPLICATIONS

Anti-Aging Applications. Mice with a disrupted growth hormone receptor/binding protein gene enjoy an increased lifespan. In U.S. Prov. Appl. 60/485,222, filed Jul. 8, 2003 (Kopchick8) mouse genes differentially expressed in comparisons of gene expression in growth hormone receptor/binding protein gene-disrupted mouse livers and normal mouse livers were identified, as were corresponding human genes and proteins. It was suggested that the human molecules, or antagonists thereof, could be used for protection against faster-than-normal biological aging, or to achieve slower-than-normal biological aging. It was also taught that the human molecules may also be used as markers of biological aging.

In provisional application Ser. No. 60/566,068, filed Apr. 29, 2004 (our docket Kopchick14-USA), our research group used a gene chip to study the genetic changes in the muscle of C57Bl/6 mice that occur at various intervals of the aging process. Differential hybridization techniques were used to identify mouse genes that are differentially expressed in mice, depending upon their age. The level of gene expression of approximately 10,000 mouse genes ( from the Amersham Codelink UniSet Mouse I Bioarray, product code: 300013)in the muscle of mice with average ages of 35, 49, 77, 118, 133, 207, 403, 558 and 725 days was determined. In essence, complementary RNA derived from mice of different ages was screened for hybridization with oligonucleotide probes each specific to a particular mouse gene, each gene in turn representative of a particular mouse gene cluster (Unigene). Mouse genes which were differentially expressed (younger vs. older), as measured by different levels of hybridization of the respective cRNA samples with the particular probe corresponding to that mouse gene, were identified. Related human genes and proteins were identified by sequence comparisons to the mouse gene or protein.

Anti-Diabetes Applications. In U.S. Provisional Appl. Ser. No. 60/458,398 (our docket Kelder1-USA), filed Mar. 31, 2003, members of our research group describe the identification of genes differentially expressed in normal vs. hyperinsulinemic, hyperinsulinemic vs. type II diabetic, or normal vs. type II diabetic mouse liver. Forward- and reverse-substracted cDNA libraries were prepared, clones were isolated, and differentially expressed cDNA inserts were sequenced and compared with sequences in publicly available sequence databases. The corresponding mouse and human genes and proteins were identified.

The purpose of our research group's provisional application Ser. No. 60/460,415 (our docket: Kopchick6-USA), filed Apr. 7, 2003, was similar, but complementary RNA, derived from RNA of mouse liver, was screened against a mouse gene chip. See also 60/506,716, filed Sep. 30, 2003 (Kopchick6.1).

Gene chip analyses have also been used to identify genes differentially expressed in normal vs. hyperinsulinemic, hyperinsulinemic vs. type II diabetic, or normal vs. type II diabetic mouse pancreas, see U.S. Provisional Appl. 60/517,376, filed Nov. 6, 2003 (Kopchick12) and muscle, see U.S. Provisional Appl. 60/547,512, filed Feb. 26, 2004 (Kopchick15).

Other differential hybridization applications. The use of differential hybridization to identify genes and proteins is also described in our research group's Ser. No. PCT/US00/12145 (Kopchick 3A-PCT), Ser. No. PCT/US00/12366 (Kopchick4A-PCT), and Ser. No. 60/400,052 (Kopchick5).

All of the foregoing applications are hereby incorporated by reference in their entirety.

### BACKGROUND OF THE INVENTION

1. Field of the Invention

The invention relates to various nucleic acid molecules and proteins, and their use in (1) diagnosing aging, or adverse conditions associated with the aging process, and (2) protecting mammals (including humans) against the aging process or adverse conditions associated with the aging process.

2. Description of the Background Art

The mechanisms that cause aging (the decline in survival and reproductive ability with advancing age) have puzzled our society and scientific community for centuries. The two major theories center on the question of whether normal aging is an evolutionarily-genetically preprogrammed pathway of internal changes or is a normal consequence of existence where there is an accumulation of molecular and cellular damages. Hypotheses of such accumulated damage include free radical-oxidative damage, defective mitochondria, somatic mutations, progressive shortening of telomeres, programmed cell death, impaired cell proliferation and numerous others (1). The current belief is that aging is not a programmed process in that, to date, no genes are known to have evolved specifically to cause damage and aging. The one factor that has been shown to extend the lifespan in organisms from yeast to mice has been a reduction in caloric intake (2, 3). Recent data suggests that caloric restriction may also be relevant for primates, including humans (4-6). Unfortunately, it is unlikely that most people will be able to maintain the strict dietary control required to reap the benefits of this finding. Therefore, since the mechanism(s) by which caloric restriction extends lifespan are unknown, the elucidation of such mechanisms could lead to the development of alternative strategies to yield similar benefits.

Numerous groups are presently engaged in identifying genes and pathways that are involved in the aging process. A growing list of genes that extend adult longevity have been identified and a large proportion of these genes are involved with hormonal signals. Many of these genes and the corresponding endocrine systems are conserved among a wide variety of eukaryotes. What is becoming clear, at least in lower animal species, is that those pathways that provide advantages to development and growth early in life may impart negative consequences in later life. The clearest example of a genetic pathway affecting adult lifespan has been described in the nematode, Caenorhabditis elegans. When food is abundant, C. elegans develops directly to the reproductive adult through four larval stages in three days. Under adverse conditions such as caloric restriction or high population density, C. elegans enters the Dauer diapause, a non-feeding, stress-resistant larval state. Genetic analysis has identified that mutation of single genes involved in dauer formation (Daf) greatly extend the adult lifespan (7). These genes involve the highly-conserved insulin/IGF-like signal transduction pathway. Ligand binging to the daf-2 insulin-like receptor results in a kinase signaling cascade to phosphorylate the forkhead transcription factor, daf-16. This phosphorylation sequesters daf-16 to the cytoplasm and results in reproductive maturity and aging. In the absence of ligand and signal transduction, the unphosphorylated, daf-16 localizes to the nucleus and regulates the transcription of its target genes that promote dauer formation, stress resistance and extended longevity (8). A similar pathway has been described in Drosophilia melanogaster. Mutation of the gene encoding insulin-like receptor (InR) or the gene encoding insulin-receptor substrate (chico) also extends the normal life-span (9, 10). Vertebrate homologues of daf-16 down-regulate genes promoting cell progression, induce genes involved in DNA-damage repair and up-regulate genes that reduce intracellular reactive oxygen species (ROS) (11, 12). A second C. elegans gene, clk-1, has also been linked to the reduction of ROS and an extended life-span. While the effect of daf-2 mutants result in a reduction of mitochondrial ROS, clk-1 mutants reduce extramitochondrially produced ROS. Since the majority of cellular ROS is produce in the mitochondria during the process of electron transport, it is not surprising that clk-1 mutants have only a moderately extended life-span. C. elegans containing daf-2/clk-1 double mutations, however, exhibit a very long life-span (13).

Decreased IGF-1 signaling may also extend longevity in mice. Four mouse models with deficiencies in pituitary endocrine action have demonstrated retarded aging. In the Prop1 and Pit1 models, pituitary production of growth hormone (GH), prolactin (PRL) and thyroid stimulating hormone (TSH) are ablated. These mice have reduced growth rates, reduced adult body size and live 40 to 60% longer than normal mice (14, 15). Unfortunately, it is not possible to determine which of the ablated hormones is responsible for the increased longevity of the models.

A more straightforward model was developed that targeted the deletion of the growth hormone receptor (GHR-KO) (16). This mouse line was derived from a founder animal by homologous recombination resulting in deletion and gene substitution of most of the fourth exon and part of the fourth intron of the GHR/BP gene. These mice also exhibit reduced body size and extended life-span and more directly implicates the GH/IGF-1 axis (17, 17a). Recently, evidence for a direct role of IGF-1 receptor signaling in affecting the aging process was provided by the targeted disruption of the IGR-1 receptor (Igf1r) (18). Heterozygous females, but not males, possess 50% fewer receptors for IGF-1, live 33% longer than wild-type females and also display greater resistance to oxidative stress. Tyrosine phosphorylation of the intracellular signaling molecule, Shc, was also decreased in the Igf1r±females. Mice containing the targeted deletion of p66shc also have increased resistance to oxidative stress and a 30% increase in life span (19). While the IGF-1 axis appears to be involved in the aging process, the mechanism by which it does so remains unknown. However, these findings demonstrate that it is possible to identify specific genetic pathways that affect the aging process. The finding that caloric restriction of these mouse models can further extend their life-span suggests that multiple-pathways exist that affect the aging process (20). Therefore, research to identify these pathways and the genes involved in the aging process is of great importance.

The role of growth hormone in aging is further discussed in Vance, M L, “Can Growth Hormone Prevent Aging,” New Engl. J. Med., 348: 779-80 (Feb. 27, 2003).

Gene-Chip Based Identification of Genes Involved in Aging of Liver

Several groups have begun to utilize DNA microarrays to measure differences in gene expression caused by the aging process. However, these experiments are extremely limited in regards to the number of aging time points or experimental conditions.

Cao, S. X., et al., “Genomic profiling of short- and long-term caloric restriction effects in the liver of aging mice”, Proc. Natl. Acad. Sci. USA, 98:10630-10635 (2001) used Affymetrix microarray technology to study the changes in expression levels of 11,000 genes in liver tissue of 7 month-old mice compared to 27 month-old mice. In this analysis, the expression of 20 genes increased at least 1.7-fold with age while the expression of 26 genes decreased at least 1.7-fold with age. We have compared the differentially expressed genes described by Cao et al., to those that we have found to be differentially expressed using the Amersham platform. Of the 20 up-regulated genes, 10 had links from Affymetrix to Amersham through Unigene. Only one of Cao's up-regulated genes, Heat shock protein (L07577/NM_{—}010410) was identified as differentially expressed in our analysis (increased 2.2-fold from weeks 2 to 4). Of Cao's 26 down-regulated genes, 10 had links from Affymetrix to Amersham through Unigene. Only one of these down-regulated genes (Mouse TIS21 gene, M64292/NM_{—}007570) was identified as differentially expressed in our analysis. However, we found the expression of this gene to increase 2.07-fold with age.

Tollet-Egnell, P., et al., “Gene expression profile of the aging process in rat liver: normalizing effects of growth hormone replacement, Mol. Endocrinol., 15(2):308-18 (2001) used microarray technology to study the effect of aging and growth hormone treatment on the expression of 3,000 different genes in the rat liver. The proteins which were over-expressed in the older rat were glucose-6-phosphate isomerase (x1.8), pyruvate kinase (x4.8), hepatic product spot 14 (2.4x), fatty acid synthase (1.9x), staryl CoA desaturase (1.7x), enoyl CoA hyydratase (1.7x), peroxisome proliferator activated receptor-α (1.7x), 3-ketoacyl-CoA thiolase (1.7x), 3-keto-acyl-CoA peroxisomal thiolase (1.9x), CYP4A3 (3.3x), glycerol-3-phosphate dehydrogenase (1.7x), NAPDH-cytochrome P450 oxidoreductase (4.7x). CUP2C7 (1.9x), CYP3A2 (2.8x), Δ-aminoevulinate synthase (2.3x). The under-expressed proteins were glucose-6-phosphatase (0.3x), farnesyl pyrophosphate synthase (0.5x), carnitine octanoyltransferase (0.5x), mitochrondrial genome (16S ribosomal RNA)(0.3x), mitochondrial cytochrome c oxidase II (0.4x), mitochondrial NADH dehydrogenase SU 5 (0.3x), mitochondrial cytochrome b (0.4x), mitochondrial NADH dhydrogenase SU 3 (0.5x), NADH-ubiquinone oxidoreductase (SU CI-SGDH and SU 39 kDa) (both 0.5x), ubiquinol-cytochrome c reductase (Rieske iron-sulfur protein and core 1) (both 0. 5x), CYP2C12 (0.4x), cystathione γ-lyase (0.3x), biphenyl hydrolase-related protein (0.5x), glutathione S-transferase (class pi)(0.3x), α-1 macroglobulin (0.5x), BRAK related protein (0.3x), α-2u-globulin (0.4x), cAMP-dependent transcription factor mATF4 (0.5x), DAP-like kinase (0.5x), PCTAIRE-1 (0.5x), collagen α-1 (0.4x), histone H2A (0.5x), and S-100 protein a (0.5x).

Of the genes up-regulated in the older rat according to Tollet-Egnall, two have mouse cognates which we found to be up-regulated in the mouse liver. These were fatty acid synthase and stearyl CoA desaturase. A third, aminoevulinate synthase, has a mouse cognate which we found to be down-regulated in the older mouse. Two genes found by Tollet-Egnall to be down-regulated in the older rat were found by us to have cognates down-regulated in the older mouse: carnitine octanoyltransferase and CYP2C12.

See also Dozmorov I, Bartke A, Miller R A., “Array-based expression analysis of mouse liver genes: effect of age and of the longevity mutant Propldf”, J. Gerontol., 56A: B52-57 (2001). Liver mRNA levels were measured in Ames dwarf mice (homozygous for the df allele at the Propl locus; live 40% to 70% longer than nonmutant siblings) and in control mice at ages 5, 13 and 22 months. “The analysis showed seven genes where the effects of age reach p<0.01 in normal mice and six others with possible age effects in dwarf mice, but none of these met Bonferroni-adjusted significance thresholds. Thirteen genes showed possible effects of the df/df genotype at p<0.01. One of these, insulin-like growth factor 1 (IGF-1), was statistically significant even after adjustment for multiple comparisons; and genes for two IGF-binding proteins, a cyclin, a heat shock protein, p38 mitogen-activated protein kinase, and an inducible cytochrome P450 were among those implicated by the survey. In young control mice, half of the expressed genes showed SDs that were more than 58% of the mean, and a simulation study showed that genes with this degree of interanimal variation would often produce false-positive findings when conclusions were based on ratio calculations alone (i.e., without formal significance testing). Many genes in our data set showed apparent young-to-old or normal-to-dwarf ratios above 2, but the large majority of these proved to be genes where high interanimal variation could create high ratios by chance alone, and only a few of the genes with large ratios achieved p<0.05. The proportion of genes showing relatively large changes between 5 and 13 months, or from 13 to 22 months of age, was not diminished by the df/df genotype, providing no support for the idea that the dwarf mutation leads to global delay or deceleration of the pace of age-dependent changes in gene expression.”

Gene-Chip Based Identification of Genes Involved in Aging of Other Organs and Tissues

Gene expression profiling has been performed on skeletal muscle tissue of mice at 5 verses 30 months of age with or without caloric restriction (21). In this analysis, the expression of 113 genes was found to be changes by at least two-fold in 5-month old mice compared to 30-month old mice. Caloric restriction of comparable mice caused a reversal of the altered gene expression of 33 genes. Similar analyses have also been performed on mouse brain and heart (22, 23).

Weindruch, et al., “Microarray profiling of gene expression in aging and its alteration by caloric restriction in mice” in Symposium: Calorie Restriction: effects on Body Composition, Insulin Signaling and Aging 918S-923S (2001) (21) compared expression in gastrocnemius muscle from 5- and 30-month old C57BL/6 mice, with and without caloric restriction. In this analysis, the expression of 113 genes was found to be changed by at least two-fold in 5-month old mice compared to 30-month old mice. Caloric restriction of comparable mice caused a reversal of the altered gene expression of 33 genes.

Of the 6347 genes surveyed in the oligonucleotide microarray, only 58 (0.9%) displayed a greater than 2 fold increase in gene expression as a function of aging, whereas 55 (0.9%) displayed a greater than 2 fold decrease. Of the genes positively correlated with aging, 16% could be assigned to stress responses. The largest differential expression between young and aged animals (3.8 fold) was the mitochondrial sarcomeric creatine kinase.

Of the genes negatively correlated with aging, 13% were involved in energy metabolism. A noteworthy number were genes encoding biosynthetic enzymes (cytochrome P450 IIC12, squalene synthase, stearoyl-CoA desaturase, EF-1-gamma. Another down regulator was a CpG binding protein, MeCP2.

Weindruch further reported that age-related changes in gene expression profile were “remarkably attenuated” by caloric restriction.

What appears to be the same experiment is discussed in Lee, et al., “Gene expression profile of aging and its retardation by caloric restriction,” Science, 285: 1390 (Aug. 27, 1999). This papers lists the individual genes which were differentially expressed by more than 2-fold, and classifies them as energy metabolism, neuronal factors, protein metabolism, stress response, biosynthesis, calcium metabolism or DNA repair genes.

Welle, et al., “Skeletal muscle gene expression profiles in 20-29 year old and 65-71 year old women,” Exper. Gerontol., 39: 369-77 (2004) and available electronically as doi:10.1016/j.exger.2003.11.011 studied gene expression and physical condition in seven young and eight older women. With respect to physical condition, the measured or calculated parameters were total body mass, lean body mass, left leg lean mass (by biopsy), maximum isometric left knee extension force, left knee extension force/left keg lean mass, Peak VO₂/lean body mass, and Peak VO₂/left leg lean mass.

There were 1178 “probe sets” (representing 1053 different Unigene clusters) for which differential expression was detected; 550 for which expression was higher in older women, and 628 the inverse effect. The differences ranged from 1.2 to 4 fold; most (78A %) were less than 1.5 fold. The complete list of differentially expressed genes is given in the Rochester Muscle database website, www.urmc.rochester.edu/smd/crc/swindex (“.html” omitted, in accordance with USPTO requirements, so that the publication of this application will not create an active hyperlink).

The gene most highly overexpressed in older muscle was p21 (cyclin-dependent kinase inhibitor 1A) (4.01 fold). This one of several genes (see Welle Table 2) which are potentially related to DNA damage and repair. Welle also thought it noteworthy how many of the differentially expressed genes were ones that encode proteins which bind to pre-mRNAs or mRNAs (see Welle Table 3).

See also Lee et al., Science, 285 :1390-93 (1999) and Nature Genetics 25: 294-7 (2000) (bioarray study of changes in mouse cerebellum and neocortex to detect age-associated genes).

Non-Gene Chip Differential/Subtractive Hybridization Studies

The papers collected in this section deal principally with type II diabetes, which is an aging-related disease.

Sreekumar, et al., “Gene expression profile in skeletal muscle of type 2 diabetes and the effect of insulin treatment,” Diabetes 51: 1913 (June 2002) surveyed 6,451 genes, and identified 85 genes for which there was an alteration in skeletal muscle transcription in diabetic patients after withdrawal of insulin treatment. Subsequent insulin treatment resulted in further changes in transcription of 74 of the 85 genes (15 increased, 59 decreased), and also resulted in alteration of 29 additional gene transcripts.

Mootha, et al., “PCG-1α responsive genes involved in oxidative phosphorylation are coordinatively downregulated in human diabetes,” Nature Genetics 34(3); 267 (July 2003), used DNA microarrays to detect changes in the expression of sets of related genes, rather than of individual genes. They classified over 22,000 genes into 149 data sets; some of these data sets overlapped. They looked for a statistical correlation between the overall rank order of the genes in differential expression, and the groups to which the genes belonged. Expression was compared pairwise among three groups: males with normal glucose tolerance; males with impaired glucose tolerance; and males with type 2 diabetes. The set with the highest enrichment score (the one whose members ranked highly most often relative to chance expectation) was an internally curated set of 106 genes involved in oxidative phosphorylation. While the average decrease for the individual genes was modest (˜20%), it was also consistent, being observed in 89% (94/106) of the genes in question. This paper is reviewed by Toye and Gauguier, “Genetics and functional genomics of type 2 diabetes mellitus”, Genome Biology, 4: 241 (2003).

Patti, et al., “Coordinated reduction of genes of oxidative metabolism in humans with insulin resistance and diabetes: Potential role of PGC1 and NRF1”, Proc. Nat. Acad. SCi. (USA), 100(14): 8466 (Jul. 8, 2003) used microarrays to analyze skeletal muscle expression of genes in nondiabetic insulin-resistant subjects at high risk for diabetes (based on family history of diabetes and Mexican-American ethnicity) and diabetic Mexican-American subjects. Of 7,129 sequences represented on the microarray, 187 were differentially expressed between control and diabetic subjects. However, no single gene remained significantly differentially expressed after controlling for multiple comparison false discovery by using the Benjamini-Hochberg method, see Benjamini, et al., J. R. Stat. Soc. Sert. B. 57:289-300 (1995); Dudait, et al., Stat. Sin. 12: 111-139 (2002). Consequently, Patti et al. sought to identify groups of related genes with similar patterns of differential expression using MAPP FINDER and ONTOEXPRESS. According to MAPP FINDER, the top-ranked cellular component terms were mitochondrion, mitochondrial membrane, mitochondrial inner membrane, and ribosome, and the top-ranked process term was ATP biosynthesis. According to ONTOEXPRESS, the over-represented groups were energy generation, protein biosynthesis/ribosomal proteins, RNA binding, ribosomal structural protein, and ATP synthase complex.

Huang, Xudong, “Identification of abnormally expressed genes in skeletal muscle contributing to insulin resistance and type 2 diabetes”, Thesis, document id: 9576 Lunds University 2002, reported differential expression of the mitochondrially-encoded ND1 gene in human diabetic patients and of the nuclear-encoded cathepsin L gene in mice.

Standaert, et al., “Skeletal muscle insulin resistance in obesity-associated type 2 diabetes in monkeys is linked to a defect in insulin activation of protein kinase C-zeta/lambda/iota Diabetes 51: 2936 (October 2002), the authors concluded that defective activation of atypical PKCs played an important role in the pathogenesis of peripheral insulin resistance in both obese prediabetic and diabetic monkeys. They attributed this linkage to the apparent requirement for aPKCs during insulin-stimulated glucose transport.

Srommer, et al., Am. J. Physiol., “Skeletal muscle insulin resistance after trauma: insulin signaling and glucose transport”, 275(2 Pt. 1): E3518(August 1998) concluded that insulin resistance in skeletal muscle after surgical trauma is associated with reduced glucose transport but not with impaired glucose signaling to PI 3-kinase or its downstream target, Akt.

Zhang, et al., Kidney International, 56 :549-558 (1999) identified genes up-regulated in 5/6 nephrectomized (subtotal renal ablation) mouse kidney by a PCR-based subtraction method. Ten known and nine novel genes were identified. The ultimate goal was to identify genes involved in glomerular hyperfiltration and hypertrophy.

Melia, et al., Endocrinol., 139:688-95 (1998) applied subtractive hybridization methods for the identification of androgen-regulated genes in mouse kidney. The treatment mice were dosed with dihydrotestosterone, an androgen. Kidney androgen-regulated protein gene was used as a positive control, as it is known to be up-regulated by DHT.

See also Holland, et al., Abstract 607, “Identification of Genes Possibly Involved in Nephropathy of Bovine Growth Hormone Transgenic Mice” (Endocrine Society Meeting, Jun. 22, 2000) and Coschigano, et al., Abstract 333, “Identification of Genes Potentially Involved in Kidney Protection During Diabetes” (Endocrine Society Meeting, Jun. 22, 2000).

The following differential hybridization articles may also be of interest: Wada, et al., “Gene expression profile in streptozotocin-induced diabetic mice kidneys undergoing glomerulosclerosis”, Kidney Int, 59:1363-73 (2001); Song, et al., “Cloning of a novel gene in the human kidney homologous to rat munc13S: its potential role in diabetic nephropathy”, Kidney Int., 53:1689-95 (1998); Page, et al., “Isolation of diabetes-associated kidney genes using differential display”, Biochem. Biophys. Res. Comm., 232:49-53 (1997); Peradi, “Subtractive hybridization claims: An efficient technique to detect overexpressed mRNAs in diabetic nephropathy,” Kidney Int. 53:926-31 (1998); Condorelli, EMBO J., 17:3858-66 (1998);

See also Nadler, S. T., Stoehr, J. P., Schueler, K. L., Tanimoto, G., Yandell, B. S., Attie, A. D. (2000) “The expression of adipogenic genes is decreased in obesity and diabetes mellitus”, Proc Natl Acad. Sci U S A 97:11371-11376; Lan H, Rabaglia M E, Stoehr J P, Nadler S T, Schueler K L, Zou F, Yandell B S, Attie A D. (2003) “Gene expression profiles of nondiabetic and diabetic obese mice suggest a role of hepatic lipogenic capacity in diabetes susceptibility”, Diabetes 52:688-700.

See also WO00/66784 (differential hybridization screening for brown adipose tissue); PCT/US00/12366, filed May 5, 2000 (differential hybridization screening for liver).

Other Anti-Aging Studies

For genes thought to have aging inhibitory activity, see generally International Longevity Center, Workshop Reports, “Longevity Genes: From Primitive Organisms to Humans,” and “Is there an ‘Anti-Aging’ Medicine?”.

Patents of possible interest include the following:

Lin, U.S. Pat. No. 6,303,768 (2001) (“Methuselah gene”)

Lippman, U.S. Pat. No. 4,695,590 (“Method for retarding aging”)

West, U.S. Pat. No. 6,368,789 (2002) (“Screening methods to identify inhibitors of telomerase activity”)

Measurement of Biological Aging

Patents of possible interest include the following:

Kojima, U.S. Pat. No. 5,000,188 (1991) (an apparatus for measuring the physiological age of a subject).

Dimri, U.S. Pat. No. 5,795,728 (1998) (“Biomarkers of cell senescence”)

Jia, U.S. Pat. No. 6,326,209 (2001) (“Measurement and quantification of 17 ketosteroid-sulfates as a biomarker of biological age”)

Articles of interest include Kayo, et al., Proc. nat. Acad. Sci. (USA) 98:5093-98 (2001); Han, et al., Mch. Ageing Dev. 115:157-74 (2000); Dozmorov, et al., J. gerontol. A Biol. Sci. Med. Sci. 56:B72-B80 (2001); Dozmorov, et al., Id., 57: B99-B108 (2002); Miller, et al., Mol. Endocrinol., 16: 2657-66 (2002).

Apoptosis and CIDE-A

Apoptosis is a form of programmed cell death that occurs in an active and controlled manner that eliminates unwanted cells. Apoptotic cells undergo an orchestrated cascade of morphological changes such as membrane blebbing, nuclear shrinkage, chromatin condensation, and formation of apoptotic bodies which there undergo phagocytosis by neighboring cells. One of the hallmarks of cellular apoptosis is the cleavage of chromosomal DNA into discrete oligonucleosomal size fragments. This orderly removal of unwanted cells minimizes the release of cellular components that may affect neighboring tissue. In contrast, membrane rupture and release of cellular components during necrosis often leads to tissue inflammation.

The process of apoptosis is highly conserved and involves the activation of the caspase cascade. Cohen, G M. (1997) Caspases: the executioners of apoptosis. Biochem. J. 326:1-16; Budihardjo, I., Oliver, H., Lutter, M., Luo, X., Wang, X. (1999) Biochemical pathways of caspase activation during apoptosis. Annnu. Rev. Cell. Dev. Biol. 15:269-290; Jacobson, N. D., Weil, M., Raff, M. C. (1997) Programmed cell death in animal development. Cell 88:347-354. Caspases are a family of serine proteases that are synthesized as inactive proenzymes. Their activation by apoptotic signals such as CLD95 (Fas) death receptor activation or tumor necrosis factor results in the cleavage of specific target proteins and execution of the apoptotic program. Apoptosis may occur by either an extrinsic pathway involving the activation of cell surface death receptors (DR) or by an intrinsic mitochondrial pathway. Yoon, J-H. Gores G. J. (2002) Death receptor-mediated apoptosis and the liver. J. Hepatology 37:400-410.

These pathways are not mutually exclusive and some cell types require the activation of both pathways for maximal apoptotic signaling. In type-I cells, death receptor activation leads to the recruitment and activation of caspases-8/10 and the rapid cleavage and activation of caspase-3 in a mitochondrial-independent manner. Hepatocytes are members of the Type-II cells in which mitochondria are essential for DR-mediated apoptosis Scaffidi, C., Fulda, S., Srinivasan, A., Friesen, C., Li, F., Tomaselli, K. J., Debatiri, K. M., Krammer, P. H., Peter, M. E. (1998) Two CD95 (APO-1/Fas) signaling pathways. EMBO J. 17:1675-1687. In this pathway, the pro-apoptotic protein Bid is truncated activated caspases-8/10 and translocates to the mitochondria. Luo, X., Budihardjo, I., Zou, H., Slaughter, C., Wang, X. (1998) Bid, a Bc12 interacting protein, mediates cytochrome c release from mitochondria in response to activation of cell surface death receptors. Cell 94:481-490; Li, H., Zhu, H., Xu, C. J., Yuan, J. (1998) Cleavage of BID by caspase 8 mediates the mitochondrial damage in the Fas pathway of apoptosis. Cell 94:491-501. This translocation leads to mitochondrial cytochrome c release and eventual activation of caspases-3 and 7 via cleavage by activated caspase-9.

One of the substrates for activated caspase-3 is the DNA fragmentation factor (DFF). DFF is composed of a 45 kDa regulatory subunit (DFF45) and a 40 kDA catalytic subunit (DFF40). Liu, X., Zou, H., Slaughter, C., Wang, X. (1997) DFF, a heterodimeric protein that functions downstream of caspase-3 to trigger DNA fragmentation during apoptosis. Cell 89:175-184. DFF45 cleavage by activated caspase-3 results in its dissociation from DFF40 and allows the caspase-activated DNAse (CAD) activity of DFF40 to cleave chromosomal DNA into oligonucleosomal size fragments. Liu, X., Li, P., Widlak, P., Zou, H., Luo, X., Garrard, W. T., Wang, X. (1998) The 40-kDa subunit of DNA fragmentation factor induces DNA fragmentation and chromatin condensation during apoptosis. Proc. Natl. Acad. Sci. USA. 95:8461-8466; Halenbeck, R., MacDonald, H., Roulston, A., Chen, T. T., Conroy, L., Williams, L. T. (1998) CPAN, a human nuclease regulated by the caspase-sensitive inhibitor DFF45. Curr Biol. 8:537-540; Nagata, S. (2000) Apoptotic DNA fragmentation. Exp. Cell Res. 256:12-8.

Recently, a novel family of cell-death-inducing DFF45-like effectors (CIDEs) have been identified that includes CIDE-A, CIDE-B and CIDE-3/FSP2. Inohara, N., Koseki, T., Chen, S., Wu, X., Nunez, G. (1998) CIDE, a novel family of cell death activators with homology to the 45 kDa subunit of the DNA fragmentation factor. EMBO J. 17:2526-2533; Danesch, U., Hoeck, W., Ringold, G. M. (1992) Cloning and transcriptional regulation of a novel adipocyte-specific gene, FSP27. CAAT-enhancer-binding protein (C/EBP) and C/EBP-like proteins interact with sequences required for differentiation-dependent expression. J. Biol. Chem. 267:7185-7193; Liang, L., Zhao, M., Xu, Z., Yokoyama, K. K., Li, T. (2003) Molecular cloning and characterization of CIDE-3, a novel member of the cell-death-inducing DNA-fragmentation-factor (DFF45)-like effector family. Biochem. J. 370:195-203.

The CIDEs contain an N-terminal domain that shares homology with the N-terminal region of DFF45 and may represent a regulatory region via protein interaction. See Inohara, supra; Lugovskoy, A. A., Zhou, P., Chou, J. J., McCarty, J. S. Li, P., Wagner, G. (1999) Solution structure of the CIDE-N domain of CIDE-B and a model for CIDE-N/CIDE-N interactions in the DNA fragmentation pathway of apoptosis. Cell 9:747-755. The family members also share a C-terminal domain that is necessary and sufficient for inducing cell death and DNA fragmentation; see Inohara supra. The overexpression of CIDE-A induces cell death that can be inhibited by DFF45. However, CIDE-A-induced apoptosis in not inhibited by caspase-8 inhibitors thereby suggesting the presence of additional, caspase-independent, pathway(s) for the induction of apoptosis, see Inohara supra. Previous reports have indicated that human and mouse CIDE-A is expressed in several tissues such as brown adipose tissue (BAT) and heart and is localized to the mitochondria, Zhou, Z., Yon Toh, S., Chen, Z., Guo, K., Ng, C. P., Ponniah, S., Lin, S. C., Hong, W., Li, P. (2003) Cidea-deficient mice have lean phenotype and are resistant to obesity. Nat. Genet. 35:49-56. In addition to the ability to induce apoptosis, CIDE-A can interact and inhibit UCP1 in BAT and may therefore play a role in regulating energy balance, see Zhou supra.

Previous reports have indicated that CIDE-A is not expressed in either adult human or mouse liver tissue, see Inohara supra, Zhou supra. We report here that CIDE-A is not only expressed in adult mouse liver tissue at older ages but is prematurely expressed in hyperinsulinemic and type-II diabetic mouse liver tissue. CIDE-A expression also correlates with liver steatosis in diet-induced obesity, hyperinsulinemia and type-II diabetes. These observations suggest an additional pathway of apoptotic cell death in NAFLD and that CIDE-A may play a role in this serious disease and potentially liver dysfunction associated with type-II diabetes.

### SUMMARY OF THE INVENTION

Our attention recently has focused on the generation of liver mRNA expression profiles and the identification of genes involved in the aging process. We have therefore explored the genetic changes in the liver of C57Bl/6 mice that occur during the aging process, observing the gene expression patterns that occur at many different time points.

Gene chips have been used to identify mouse genes that are differentially expressed in mice, depending upon their age. We have utilized the Amersham product code: 300013 Codelink UniSet Mouse I Bioarray to determine the level of gene expression-of approximately 10,000 mouse genes in the liver of mice with average ages of 35, 49, 77, 118, 133, 207, 403, 558 and 725 days.

In essence, complementary RNA derived from mice of different ages was screened for hybridization with oligonucleotide probes each specific to a particular mouse database DNA, as identified, by database accession number, by the gene manufacturer. Each database DNA in turn was also identified by the gene chip manufacturer as representative of a particular mouse gene cluster (Unigene).

In most cases, this database DNA sequence was a full length genomic DNA or cDNA sequence, and are therefore either identical to, or encode the same protein as does, a natural full-length genomic DNA protein coding sequence. Those which don't at least present a partial sequence of a natural gene or its cDNA equivalent.

For the sake of simplicity, all of these mouse database DNA sequences, whether full-length or partial, and whether cDNA or genomic DNA, are referred to herein as “mouse genes”. When only the genomic sequence is intended, we will refer specifically to “genomic DNA” or “gDNA”.

The sequences in the protein databases are determined either by directly sequencing the protein or, more commonly, by sequencing a DNA, and then determining the translated amino acid sequence in accordance with the Genetic Code. All of the mouse sequences in the mouse polypeptide database are referred to herein as “mouse proteins” regardless of whether they are in fact full length sequences.

Mouse genes which were substantially differentially expressed (younger vs. older), as measured by different levels of hybridization of the respective cRNA samples with the particular probe corresponding to that mouse gene, were identified.

Favorable behavior is when expression decreases with age. Substantially favorable behavior is when the ratio of younger value to older value is at least two fold. Unfavorable behavior is when expression increases with age. Substantially unfavorable behavior is when the ratio of older value to younger value is at least two fold.

A mouse gene is considered to be “favorable” (more precisely, “wholly favorable”) for the purpose of the Master Tables, especially subtable 1A, if, for at least one of the time comparisons set forth in the Examples, it exhibited substantially favorable behavior, and if, for all the other comparisons, it at least did not exhibit substantially unfavorable behavior. Note that the classification of a gene as favorable for purpose of the Master Table does not mean that it must have exhibited substantially favorable behavior for all of the comparisons set forth in the Examples.

A mouse gene is considered to be “unfavorable” (more precisely, “wholly unfavorable) for the purpose of the Master Tables, especially subtable 1B, if, for at least one of the time comparisons set forth in the Examples, it exhibited substantially unfavorable behavior, and if, for all the other comparisons, it at least did not exhibit substantially favorable behavior.

A mouse gene is considered to be “mixed” (in effect, both partially favorable and partially unfavorable) for the purpose of the Master Tables, especially subtable 1C, if for at least one of the time comparisons set forth in the Examples it exhibited substantially favorable behavior and if for at least one of the other such comparisons it exhibited substantially unfavorable behavior.

The expression of a gene may first rise, then fall, with increasing age. Or it may first fall, and then rise. These are just the two simplest of several possible “mixed” expression patterns.

Thus, we can subdivide the “favorables” into wholly and partially favorables. Likewise, we can subdivide the unfavorables into wholly and partially unfavorables. The genes/proteins with “mixed” expression patterns are, by definition, both partially favorable and partially unfavorable. In general, use of the wholly favorable or wholly unfavorable genes/proteins is preferred to use of the partially favorable or partially unfavorable ones.

It is evident from the foregoing that mixed genes/proteins are those exhibiting a combination of favorable and unfavorable behavior. A mixed gene/protein can be used as would a favorable gene/protein if its favorable behavior outweighs the unfavorable. It can be used as would an unfavorable gene/protein if its unfavorable behavior outweighs the favorable. Preferably, they are used in conjunction with other agents that affect their balance of favorable and unfavorable behavior. Use of mixed genes/proteins is, in general, less desirable than use of purely favorable or purely unfavorable genes/proteins.

It will be appreciated that the comparisons set forth in the Examples are not exhaustive and that it is possible that a mouse gene which, on the basis of those comparisons, was classified as a “favorable” gene in the Master Table may turn out, if additional time points are considered, to sometimes exhibit substantially unfavorable behavior. Nonetheless, such a gene will still be considered a “favorable” gene for the purpose of the Master Table and the claims referring to the Master Table. Likewise, a gene which, on the basis of those comparisons, was classified as an “unfavorable” gene in the Master Table may prove, under more detailed examination, to sometimes exhibit substantially favorable behavior. Nonetheless, it will retain “unfavorable” classification for the purpose of the Master Table and the claims referring thereto.

The “favorable”, “unfavorable” and “mixed” mouse proteins are thus those listed in the Master Table as encoded by the listed “favorable”, “unfavorable” and “mixed” mouse genes, respectively, or which otherwise correspond to those mouse genes.

Related human genes (database DNAs) and proteins were identified by searching a database comprising human DNAs or proteins for sequences corresponding to (i.e., homologous to, i.e., which could be aligned in a statistically significant manner to) the mouse gene or protein. The “favorable”, “unfavorable” and “mixed” human genes and proteins are those which correspond to the listed “favorable”, “unfavorable” and “mixed” mouse genes and proteins, respectively. More than one human protein may be identified as corresponding to a particular mouse chip probe and to a particular mouse gene.

Note that the terms “human genes” and “human proteins” are used in a manner analogous to that already discussed in the case of “mouse genes” and “mouse proteins”, e.g., the “genes” include both gDNA and cDNA, and both full and partial sequences.

As used herein, the term “corresponding” does not mean identical, but rather implies the existence of a statistically significant sequence similarity, such as one sufficient to qualify the human protein or gene as a homologous protein or DNA as defined below. The greater the degree of relationship as thus defined (i.e., by the statistical significance of each alignment used to connect the mouse chip DNA, and the corresponding mouse gene/cDNA, to the human protein or gene, measured by an E value), the more close the correspondence. The connection may be direct (mouse gene to human protein) or indirect (e.g., mouse gene to human gene, human gene to human protein). By “mouse gene”, we mean the mouse gene from which the gene chip DNA in question was derived.

In general, the human genes/proteins which most closely correspond, directly or indirectly, to the mouse genes are preferred, such as the one(s) with the highest, top two highest, top three highest, top four highest, top five highest, and top ten highest E values for the final alignment in the connection process. The human genes/proteins deemed to correspond to our mouse genes are identified in the Master Tables.

Note that it is possible to identify homologous full-length human genes and proteins, if they are present in the database, even if the query mouse DNA or protein sequence is not a full-length sequence.

If there is no homologous, full-length human gene or protein in the database, but there is a partial one, the latter may nonetheless be useful. For example, a partial protein may still have biological activity, and a molecule which binds the partial protein may also bind the full-length protein so as to antagonize a biological activity of the full-length protein. Likewise, a partial human gene may encode a partial protein which has biological activity, or the gene may be be useful in the design of a hybridization probe or in the design of a therapeutic antisense DNA.

The partial genes and protein sequences may of course also be used in the design of probes intended to identify the full length gene or protein sequence.

Agents which bind the “favorable” and “unfavorable” nucleic acids (e.g., the agent is a substantially complementary nucleic acid hybridization probe), or the corresponding proteins (e.g., an antibody vs. the protein) may be used to estimate the biological age of a human subject, or to predict the rate of biological aging in a human subject (i.e, to evaluate whether a human subject is at increased or decreased risk for faster-than-normal biological aging.) A subject with one or more elevated “unfavorable” and/or one or more depressed “favorable” genes/proteins is at increased risk, and one with one or more elevated “favorable” and/or one or more depressed “unfavorable” genes/proteins is at decreased risk.

The assay may be used as a preliminary screening assay to select subjects for further analysis, or as a formal diagnostic assay.

The identification of the related genes and proteins may also be useful in protecting humans against faster-than-normal or even normal aging (hereinafter, “the disorders”). They may be used to reduce a rate of biological aging in the subject, and/or delay the time of onset, or reduce the severity, of an undesirable age-related phenotype in said subject, and/or protect against an age-related disease.

Thus, Applicants contemplate:

(1) use of the “favorable” mouse DNAs (or fragments thereof) of the Master Tables (below) to isolate or identify related human DNAs;

(2) use of human DNAs, related to favorable mouse DNAs, to express the corresponding human proteins;

(3) use of the corresponding human proteins (and mouse proteins, if biologically active in humans), to protect against the disorder(s);

(4) use of the corresponding mouse or human proteins, or nucleic acid probes derived from the mouse or human genes, in diagnostic agents, in assays to measure or predict biological aging or the rate thereof; and

(5) use of the corresponding human or mouse genes therapeutically in gene therapy, to protect against the disorder(s).

Moreover Applicants contemplate:

(1) use of the “unfavorable” mouse DNAs (or fragments thereof) of the Master Tables to isolate or identify related human DNAs;

(2) use of the complement to the “unfavorable” mouse DNAs or related human DNAs, as antisense molecules to inhibit expression of the related human DNAs;

(3) use of the mouse or human DNAs to express the corresponding mouse or human proteins;

(4) use of the corresponding mouse or human proteins, in diagnostic agents, to measure biological aging or the rate thereof;

(5) use of the corresponding mouse or human proteins in assays to determine whether a substance binds to (and hence may neutralize) the protein; and

(6) use of the neutralizing substance to protect against the disorder(s).

Thus, DNAs of interest include those which specifically hybridize to the aforementioned mouse or human genes, and are thus of interest as hybridization assay reagents or for antisense therapy. They also include synthetic DNA sequences which encode the same polypeptide as is encoded by the database DNA, and thus are useful for producing the polypeptide in cell culture or in situ (i.e., gene therapy). Moreover, they include DNA sequences which encode polypeptides which are substantially structurally identical or conservatively identical in amino acid sequence to the mouse and human proteins identified in the Master Table 1, subtables 1A or 1C, and DNA sequences which encode human proteins which are members of human protein classes set forth in master table 2, subtables 2A or 2C. Finally, they include DNA sequences which peptide (including antibody) antagonists of the proteins of Master Table 1, subtables 1B or 1C, or of human proteins which are members of human protein classes set forth in master table 2, subtables 2B or 2C.

Related human DNAs also may be identified by screening human cDNA or genomic DNA libraries using the mouse gene of the Master Table, or a fragment thereof, as a probe.

If the mouse gene of Master Table 1 is not full-length, and there is no closely corresponding full-length mouse gene in the sequence databank, then the mouse DNA may first be used as a hybridization probe to screen a mouse CDNA library to isolate the corresponding full-length sequence. Alternatively, the mouse DNA may be used as a probe to screen a mouse genomic DNA library.

The human protein cell death activator CIDE-A is of particular interest because of its highly dramatic change in liver expression with age.

The agents of the present invention may be used alone or in conjunction with each other and/or known anti-aging or anti-age-related disease agents. It is of particular interest to use the agents of the present invention in conjunction with an agent disclosed in one of the related applications cited above.

### BRIEF DESCRIPTION OF THE DRAWINGS

FIG. 1 CIDE-A Expression is elevated in older normal mice. CIDE-A expression is plotted for normal C57Bl/6J mouse ages 35, 49, 56, 77, 133, 207, 403, 558 and 725 days. Expression is low for the first few data points, then rises sharply at 403 days, and again at 558 days. There is a drop off at 725 days, but expression remains above the 403 day level.

FIG. 2 CIDE-A Expression is elevated at an earlier age in diabetic mice. In diabetic mice, the CIDE-A expression at 133 days is more than double that at 77 days, while in normal mice, the increase over the same interval is slight.

FIG. 3. Steatosis in liver of high-fat diet fed mice. Mice were weaned directly onto either a normal diet or a high-fat diet and maintained on the respective diets for up to 26 weeks. The mice were sacrificed and liver tissue isolated. Percent liver white space was determined.

### DETAILED DESCRIPTION OF THE PREFERRED EMBODIMENTS OF THE INVENTION

Full-Length vs. Partial Length Genes/Proteins

A “full length” gene is here defined as a (1) a naturally occurring DNA sequence which begins with an initiation codon (almost always the Met codon, ATG), and ends with a stop codon in phase with said initiation codon (if introns, if any, are ignored), and thereby encodes a naturally occurring polypeptide with biological activity, or a naturally occurring precursor thereof, or (2) a synthetic DNA sequence which encodes the same polypeptide as that which is encoded by (1). The gene may, but need not, include introns.

A “full-length” protein is here defined as a naturally occurring protein encoded by a full-length gene, or a protein derived naturally by post-translational modification of such a protein. Thus, it includes mature proteins, proproteins, preproteins and preproproteins. It also includes substitution and extension mutants of such naturally occurring proteins.

Subjects

For mice, infancy is defined as the period 0 to 21 days after birth. Sexual maturity is reached, on average, at 42 days after birth. The average lifespan is 832 days.

In humans, infancy is defined as the period between birth and two years of age. Sexual maturity in males can occur between 9 and 14 years of age while the average age at first menstrual period for females 15-44 years old is 12.6 years. The average human lifespan is 73 years for males and 79 years for females. The maximum verified human lifespan was 122 years, five months and 14 days.

Chronological and Biological Aging

“Aging” is a process of gradual and spontaneous change, resulting in maturation through childhood, puberty, and young adulthood and then primarily a decline in function through middle and late age. Aging thus has both the positive component of development/maturation and the negative component of decline.

“Senescence” refers strictly to the undesirable changes that occur as a result of post-maturation aging. Some of the changes which occur in post-maturation aging are not deleterious to health (e.g., gray hair, baldness), and some may even be desirable (e.g., increased wisdom and experience). In contrast, the memory impairment that occurs with age is considered senescence. However, we will hereafter use “aging” per se to refer to “senescence”, and use “maturation” to refer to pre-maturation development.

There is increased mortality with age after maturation. There is also a progressive decrease in physiological capacity with age, but the rate of physiological decline varies from organ to organ and from individual to individual. The physiological decline results in a reduced ability to respond adaptively to environmental stimuli, and increased susceptibility and vulnerability to disease.

“Aging is the accumulation of diverse adverse changes that increase the risk of death. These changes can be attributed to development, genetic defects, the environment, disease, and the inborn aging process. The chance of death at a given age serves as a measure of the number of accumulated changes, that is, of physiologic age, and the rate of change of this measure, as the rate of aging.” Harman, Ann. N.Y. Acad. Sci. 854:1-7 (1998).

Preferably, the agents of the present invention inhibit aging for at least a subpopulation of mature (post-puberty) adult subjects.

The term “healthy aging” (sometimes called “successful aging”) refers to post-maturation changes in the body that occur with increasing age even in the absence of an overt disease. However, increased age is a risk factor for many diseases (“age-related diseases”), and hence “total aging” includes both the basal effects of healthy aging and the effects of any age-related disease. (Most literature uses the term “normal aging” as a synonym for “healthy aging”, but a minority use it to refer to “total aging”. To minimize confusion, we will try to avoid the term “normal aging”, but if we use it, it is as a synonym for “healthy aging”.) Some scientists have suggested that normal aging changes should be defined as those which are universal, degenerative, progressive and intrinsic.

Preferably, the agents of the present invention inhibit healthy aging for at least a subpopulation of mature (post-puberty) adult subjects.

In both aging and senescence, many physiologic functions decline, but normal decline is not usually considered the same as disease. The distinction between normal decline and disease is often but not always clear and may be due only to statistical distribution. Glucose intolerance is considered consistent with healthy aging, but diabetes is considered a disease, although a very common one. Cognitive decline is nearly universal with advanced age and is considered healthy aging; however, cognitive decline consistent with dementia, although common in late life, is considered a disease (as in the case of Alzheimer's, a conclusion supported by analysis of brain tissue at autopsy). A decline in maximal heart rate is typical of healthy aging. In contrast, coronary heart disease is an age-related disease. A decline in bone density is considered healthy aging, but when it drops to 2.5 SD below the young adult mean, it is called osteoporosis. Generally speaking, the changes typical of healthy aging are gradual, while those typical of a disorder can be rapid.

The term average (median) “lifespan” is the chronological age to which 50% of a given population survive. The maximum lifespan potential is the maximum age achievable by a member of the population. As a practical matter, it is estimated as the age reached by the longest lived member (or former member) of the population. The (average) life expectancy is the number of remaining years that an individual of a given age can expect to live, based on the average remaining lifespans of a group of matched individuals.

The most widely accepted method of measuring the rate of aging is by reference to the average or the maximum lifespan. If a drug treatment achieves a statistically significant improvement in average or maximum lifespan in the treatment group over the control group, then it is inferred that the rate of aging was retarded in the treatment group. Similarly, one can compare long-term survival between the two groups.

Preferably, the agents of the present invention have the effect of increasing the average lifespan and/or the maximum lifespan for at least a subpopulation of mature (post-puberty) adult subjects. This subpopulation may be defined by sex and/or age. If defined in part by age, then it may be defined by a minimum age (e.g., at least 30, at least 40, at least 50, at least 55, at least 60, at least 65, at least 70, at least 75, at least 80, at least 90, etc.) or by a maximum age (not more than 40, not more than 50, not more than 55, not more than 60, not more than 65, not more than 70, not more than 75, not more than 80, not more than 90, not more than 100, etc.), or by a rational combination of a minimum age and a maximum age so as to define a preferred close-ended age range, e.g., 55-75.

The subpopulation may additionally be defined by race, e.g., caucasian, negroid or oriental, and/or by ethnic group, and/or by place of residence (e.g., North America, Europe).

The subpopulation may additionally be defined by non-age risk factors for age-associated diseases, e.g., by blood pressure, body mass index, etc.

Preferably, the subpopulation in which an agent of the present invention is reasonably expected to be effective is large, e.g., in the United States, preferably at least 100,000 individuals, more preferably at least 1,000,000 individuals, still more preferably at least 10,000,000, even more preferably at least 20,000,000, most preferably at least 40,000,000.

By way of comparison, according to the 2000 U.S. LO Census, the U.S. population, by age, was

| Age | Pop (mil) |
|---|---|
| 15-19 | 20.2 |
| 20-24 | 19.0 |
| 25-29 | 19.4 |
| 30-34 | 20.5 |
| 35-39 | 22.7 |
| 40-44 | 22.4 |
| 45-49 | 20.1 |
| 50-54 | 17.6 |
| 55-59 | 13.5 |
| 60-64 | 10.8 |
| 65-69 | 9.5 |
| 70-74 | 8.9 |
| 75-79 | 7.4 |
| 80-84 | 4.9 |
| 85+ | 4.2 |

For any given chronological age, statisticians can define the probability of living to a particular later age. These expectancies can be calculated for the entire age cohort, or broken down by sex, race, country of residence, etc. Individuals who live longer than expected can be said, after the fact, to have biologically aged more slowly than their peers. One definition of biological age is that it is a measure of one's position in one's life span, i.e., biological age =position in own life span (as fraction in range 0.1) X average life span for species. This simple definition carries with it the implicit assumption that the rate of biological aging is constant. It also has the practical problem of determining one's own life span before death. We will present a more practical definition shortly.

The problem with lifespan studies is that they are extremely time-consuming. A maximum lifespan study in mice can take 4-5 years. A maximum lifespan study in dogs or cats would take 15-20 years, in monkeys, 30-40 years, and in humans, over 100 years. Even if the human study group were of sexagenarians, it would take 40-60 years to complete the study.

Hence, scientists have sought to identify biological markers (biomarkers) of biological aging, that is, characteristics that can be measured while the subjects are still alive, which correlate to lifespan. These biological markers can be used to calculate a “biological age” (syn. “Physiological age”); it is the chronological age at which an average member of the population (or relevant subpopulation) would have the same value of a biomarker of biological aging (or the same value of a composite measure of biomarkers of biological aging) as does the subject. This is the definition that will be used in this disclosure, unless otherwise stated.

The effect of aging varies from system to system, organ to organ, etc. For example, between ages 30 and 70 years, nerve conduction velocity decreases by only about 10%, but renal function decreases on average by nearly 40%. Thus, there isn't just one biological age for a subject. By a suitable choice of biomarker, one may obtain a whole organism, or a system-, organ- or tissue-specific measure of biological aging, e.g., one can say that a person has the nervous system of a 30 year old but the renal system of a 60 year old. Biomarkers may measure changes at the molecular, cellular, tissue, organ, system or whole organism levels.

Generally speaking, in the absence of some form of intervention (drugs, diet, exercise, etc.), biological ages will increase with time. The agents of the present invention preferably reduce the time rate of change of a biological age of the subject. The term “a biological age” could refer to the overall biological age of the subject, to the biological age of a particular system, organ or tissue of that subject, or to some combination of the foregoing. More preferably, the agents of the present cannot only reduce the rate of increase of a biological age of the subject, but can actually reduce a biological age of the subject.

A simple biologic marker (biomarker) is a single biochemical, cellular, structural or functional indicator of an event in a biologic system or sample. A composite biomarker is a mathematical combination of two or more simple biomarkers. (Chronological age may be one of the components of a composite biomarker.)

A plausible biomarker of biological age would be a biomarker which shows a cross-sectional and/or longitudinal correlation with chronological age. Nakamura suggests that it is desirable that a biomarker show (a) significant cross-sectional correlation with chronological cage, (b) significant longitudinal change in the same direction as the cross-sectional correlation, (c) significant stability of individual differences, and (d) rate of age-related change proportional to differences in life span among related species. Cp. Nakamura, Exp Gerontol. 29(2):151-77 (1994), using desiderata (a)-(c). A superior biomarker of biological age would be a better predictor of lifespan than is chronological age (preferably for a chronological age at which 90% of the population is still alive).

The biomarker preferably also satisfies one or more of the following desiderata: a statistically significant age-related change is apparent in humans after a period of at most a few years; not affected dramatically by physical conditioning (e.g., exercise), diet, and drug therapy (unless it is possible to discount these confounding influences, e.g., by reference to a second marker which measures them); can be tested repeatedly without harming the subject; works in lab animals as well as humans; simple and inexpensive to use; does not alter the result of subsequent tests for other biomarkers if it is to be used in conjunction with them; monitors a basic process that underlies the aging process, not the effects of disease.

Preferably, if the biomarker works in lab animals, there is a statistically significant difference in the value of the biomarker between groups of food-restricted and normally-fed animals. It has been shown in some mammalian species that dietary restriction without malnutrition (e.g., caloric decrease of up to 40% from ad libitum feeding) increases lifespan.

A biomarker of aging may be used to predict, instead of lifespan, the “Healthy Active Life Expectancy” (HALE) or the “Quality Adjusted Life Years” (QALY), or a similar measure which takes into account the quality of life before death as well as the time of death itself. For HALE, see Jagger, in Outcomes Assessment for Healthcare in Elderly People, 67-76 (Farrand Press: 1997). For QALY, see Rosser R M. A health index and output measure, in Stewart S R and Rosser R M (eds) Quality of Life: Assessment and Application. Lancaster: MTP, 1988.

A biomarker of aging may be used to predict, instead of lifespan, the timing and/or severity of a change in one or more age-related phenotypes as described below.

A biomarker of aging may be used to estimate, rather than overall biological age for a subject, a biological age for a specific body system or organ. The determination of the biological age of the liver, and the inhibition of biological aging of the liver, are of particular interest.

Body systems include the nervous system (including the brain, the sensory organs, and the sense receptors of the skin), the cardiovascular system (includes the heart, the red blood cells and the reticuloendothelial system), the respiratory system, the gastrointestinal system, the endocrine system (pituitary, thyroid, parathyroid and adrenal glands, gonads, pancreas, and parganglia), the musculoskeletal system, the urinary system (kidneys, bladder, ureters, urethra), the reproductive system and the immune system (bone marrow, thymus, lymph nodes, spleen, lymphoid tissue, white blood cells, and immunoglobuline). A biomarker may be useful in estimating the biological age of a system because the biomarker is a chemical produced by that system, because it is a chemical whose activity is primarily exerted within that system, because it is indicative of the morphological character or functional activity of that system, etc. A given biomarker may be thus associated with more than one system. In a like manner, a biomarker may be associated with the biological age, and hence the state, of a particular organ or tissue.

The prediction of lifespan, or of duration of system or organ function at or above a particular desired level, may require knowledge of the value of at least one biomarker of aging at two or more times, adequately spaced, rather than of the value at a single time. See McClearn, Biomarkers of Age and Aging, Exp. Gerontol., 32:87-94 (1997).

The levels (or changes in levels) of the human proteins identified in this specification, and their corresponding mRNAs, may be used as simple biomarkers (direct or inverse) of biological aging. They may be used in conjunction with each other, or other simple biomarkers, in a composite biomarker.

Once several plausible simple biomarkers have been identified, a composite biomarker may be obtained by standard mathematical techniques, such as multiple regression, principal component analysis, cluster analysis, neural net analysis, and so forth. As a preliminary to such analysis, the values may be standardized, e.g., by converting the raw scores into z-scores based on the distributions for each simple biomarker.

For example, principal component analysis can be used to analyze the variation of lifespan with different observables, and the factor score coefficients from the first principal component can be used to derive an equation for estimating a biological age score. Nakamura, Exp Gerontol. 29(2):151-77 (1994). This approach was used to obtain the following BAS (for healthy Japanese women aged 28-80): BAS=−4.37 −0.998FEV_{1.0}+0.022SBP +0.133MCH +0.018GLU −1.505 A/G RATIO, where FEV_{1.0} is the forced expiratory volume in 1 sec. (Liters), SBP is the systolic blood pressure (mm Hg), MCH is the mean corpuscular hemoglobin (pg), GLU is glucose (mg/dl), and A/G RATIO is the ratio of albumin to globulin. The relative importance of these five biomarkers was 33.7%, 25.1%, 17.1%, 14.8% and 8.9%, respectively. Ueno, et al., “Biomarkers of Aging in Women and the Rate of Longitudinal Changes,” J. Physiol. Anthropol. 22(1): 37-46 (January 2003).

It should be noted that particularly when evaluating the overall biological age of the subject, it is not necessarily most desirable to weight all systems or all organs equally. One may find it more desirable to give greater weight to the system or organ with the highest biological age in calculating the overall biological age, because it is presumably more likely to deteriorate or fail, resulting in death. Appropriate statistical analysis can be used to find the weighting scheme resulting in the best prediction of lifespan.

In the H-SCAN (Hoch Company) test, a composite of 12 simple biomarkers is used to measure human aging:

Sensory

1. Highest audible pitch (kHz)
2. Visual accommodation (diopters)
3. Vibrotactile sensitivity (dB)
   Motor
4. Muscle Movement time (sec)
5. Muscle Movement time with decision (sec)
6. Alternate button tapping time (sec)
   Cognitive
7. Memory, length of sequence
8. Auditory reaction time (sec)
9. Visual reaction time (sec)
10. Visual Reaction time with decision (sec)
   Pulmonary
11. Forced vital capacity (liters)
12. Forced expiratory Volume-1 sea (liters)
   See Hochschild, R., Journal of Gerontology [Biolcgical Science] 45(6):B187-214; 1990).
According to a website discussing the H-SCAN test, “Biomarkers of aging are characteristics of an organism that correlate in large groups with chronological age and mortality. Of particular value in human applications are biomarkers of aging that also correlate with the duality of life in later life in the sense that they involve functions that are crucial to carrying out the activities of daily living . . . . A single biomarker of aging is limited by the fact that it measures only one isolated characteristic and is hardly representative of the diversity of functional and structural concomitants of aging . . . . Biological age, in contrast to chronological age, is an individual's hypothetical age calculated from scores obtained on a battery of tests of biomarkers of aging. As a first step in the calculation, the age of which each biomarker score is typical is determined by comparison with scores obtained by a large representative group of persons (or organisms) spanning a range of ages. Then one of a variety of averaging techniques is employed (optionally with standardization steps) to obtain a single index of age, as described in detail by Hochschild. This index varies with, and therefore must be expressed with reference to, the measured biomarkers and the mathematical method of combining scores.”http://www.longevityinstituteone.com/
Abbo, U.S. Pat. No. 6,547,729 teaches determining the biological age (he calls it “performance age”) of a subject by (1) for a sample population, determining a regression curve relating some set of observed values for an “indicator” of the functionality of a bodily system to the chronological age of the observed individuals, (2) solving the regression equation to obtain a predicted performance age, given the value of the indicator for the subject. The regression can be based on more than one indicator, i.e., it can be a multiple regression. The sample population can be defined by sex, age range, ethnic composition, and geographic location. The bodily system may be a molecular, cellular, tissue or organ system. The following indicators are suggested by Abbo: nervous system (memory tests, reaction time, serial key tapping, digit recall test, letter fluency, category fluency, nerve conduction velocity), arteries (pulse wave velocity; ankle-brachial index), skeletal system (bone mineral density); lungs (forced vital capacity), heart (ejection fraction; length of time completed on a treadmill stress test), kidneys (creatinine clearance), proteins (glycosylation of hemoglobin), endocrine glands (load level of bioactive testosterone; level of dehydroepiandrosterone sulfate, ratio of urinary 17-ketosteroids/17-hydroxycorticosteroids; growth hormone; IGF-1).

Preferably, the agents of the invention have a favorable effect on the value of at least one simple biomarker of biological aging, such as any of the plausible biomarkers mentioned anywhere in this specification, other than the level of one of the proteins of the present invention. More preferably, they have a favorable effect on the value of at least two such simple biomarkers of biological aging. Even more preferably, at least one such pair is of markers which are substantially non-correlated (R²<0.5)

Desirably, if more than one simple biomarker is favorably affected, the biomarkers in question reflect different levels of organization, and/or different body components at the same level of organization. For example, a visual reaction time with decision test is on the whole organism level, while a measurement of telomere length is on the a cellular level.

A biomarker may, but need not, be an indicator related to one of the postulated causes or contributing factors of aging. It may, but need not, be an indicator of the acute health of a particular body system or organ.

A biomarker may measure behavior, cognitive or sensory function, or motor activity, or some combination thereof.

It may measure the level of a type of cell (e.g., a T cell subset, such as CD4, CD4 memory, CD4 naive, and CD4 cells expressing P-glycoprotein) or of a particular molecule (e.g., growth hormone, IGF-1, insulin, DHEAS, an elongation factor, melatonin) or family of structurally or functionally related molecules in a particular body fluid (especially blood) or tissue. For example, lower serum IGF-1 levels are correlated with increasing age, and IGF-1 is produced by many different tissues. On the other hand, growth hormone is produced by the pituitary gland.

A biomarker may measure an indicator of stress (particularly oxidative stress) and resistance thereto. It has been theorized that free radicals damage biomolecules, leading to aging.

A biomarker may measure protein glycation or other protein modification (e.g., collagen crosslinking). It has been theorized that such modifications contribute to aging.

The biomarker may measure changes in the lengths of telomeres or in the rate of cell division. It has been theorized that telomere shortening beyond a critical length leads the cell to stop proliferating. Average telomere length therefore provides a biomarker as to how may divisions the cell as previously undergone and how many divisions the cell can undergo in the future.

Suggested biomarkers have also included resting heart rate, resting blood pressure, exercise heart rate, percent body fat, flexibility, grip strength, push strength, abdominal strength, body temperature, and skin temperature.

The present invention does not require that all of the biomarkers identified above be validated as indicative of biological age, or that they be equally useful as measures of biological age.

There is an overlap between biomarkers of aging and indicators of functional status. An indicator of functional status is an indicator that defines a functional ability (e.g., physiological, cognitive or physical function). An indicator of functional status may also be related to the increase in morbidity and mortality with chronological age. Such indicators preferably predict physiological, cognitive and physical function in an age-coherent way, and do so better than chronological age. Preferably, they can predict the years of remaining functionality, and the trajectory toward organ-specific illness in the individual. Also, they are preferably minimally invasive.

Suggested indicators include anthropometric data (body mass index, body composition, bone density, etc.), functional challenge tests (glucose tolerance, forced vital capacity), physiological tests (cholesterol/HDL, glycosylated hemoglobin, homocysteine, etc.) and proteomic tests.

A number of mouse models for human aging exist. See Troen, supra, Table 3. The drugs identified by the present invention may be further screened in one or more of these models.

Age-Related Phenotype

An age-related phenotype is an observable change which occurs with age. An age-related phenotype may, but need not, also be a biomarker of biological aging

Preferably, the agent of the present invention favorably affects at least one age-related phenotype. More preferably, it favorably affects at least two age-related phenotypes, more preferably phenotypes of at least two different body systems.

The age-related phenotype may be a system level phenotype, such as a measure of the condition of the nervous system, respiratory system, immune system, circulatory system, endocrine system, reproductive system, gastrointestinal system, or musculoskeletal system.

The age-related phenotype may be an organ level phenotype, such as a measure of the condition of the brain, eyes, ears, lungs, spleen, heart, pancreas, liver, ovaries, testicles, thyroid, prostate, stomach, intestines, or kidney.

The age-related phenotype may be a tissue level phenotype, such as a measure of the condition of the muscle, skin, connective tissue, nerves, or bones.

The age-related phenotype may be a cellular level phenotype, such as a measure of the condition of the cell wall, mitochondria or chromosomes.

The age-related phenotype may be a molecular level phenotype, such as a measure of the condition of nucleic acids, lipids, proteins, oxidants, and anti-oxidants.

The age-related phenotype may be manifested in a biological fluid, such as blood, urine, saliva, lymphatic fluid or cerebrospinal fluid. The biochemical composition of these fluid may be an overall, system level, organ level, tissue level, etc. phenotype, depending on the specific biochemical and fluid involved.

PHYSIOLOGICAL AGING OF THE HUMAN BODY BY SYSTEMS

| | |
|---|---|
| SKIN, HAIR, | Loss of subcutaneous fat, Thinning of skin, |
| NAILS | Decreased collagen, Nails brittle and flake, |
| | Mucous membranes drier, Less sweat glands, |
| | Temperature regulation difficult, Hair |
| | pigment decreases, Hair thins. Eyelids baggy |
| | and wrinkled. |
| EYES AND | Eyes deeper in sockets; Conjunctiva thinner |
| VISION | and yellow; Quantity of tears decreases; Iris |
| | fades; Pupils smaller, let in less light; |
| | Night and depth vision less; “Floaters” can |
| | appear |
| | Lens enlarges; Lens becomes less transparent, |
| | can actually become clouded, results in |
| | cataracts; Accommodation decreases, results |
| | in presbyopia; Impaired color vision, also - |
| | especially greens and blues- because cones |
| | degenerate; Predisposed to glaucoma |
| | (Increased pressure in eye, decreased |
| | absorption of intraocular fluid; can result |
| | in blindness); |
| | Macular degeneration becoming more frequent |
| | (This is the patch of retina where lens |
| | focuses light, Ultimately results in |
| | blindness) |
| EARS AND | Irreversible, sensorineural loss |
| HEARING LOSS | (presbycusis) with age (Men more affected |
| | than women, Loss occurs in higher range of |
| | sound, By 60 years, most adults have trouble |
| | hearing above 4000 Hz, Normal speech |
| | 500-2000 Hz) |
| RESPIRATORY | Lungs become more rigid, Pulmonary function |
| SYSTEM | decreases, Number and size of alveoli |
| | decreases, Vital capacity declines, Reduction |
| | in respiratory fluid, Bony changes in chest |
| | cavity |
| CARDIOVASCULAR | Heart smaller and less elastic with age, By |
| SYSTEM | age 70 cardiac output reduced 70%, Heart |
| | valves become sclerotic, Heart muscle more |
| | irritable, More arrhythmias, Arteries more |
| | rigid, Veins dilate |
| GASTROINTESTINAL | Reduced GI secretions, Reduced GI motility, |
| SYSTEM | Decreased weight of liver, |
| | Reduced regenerative capacity of liver, Liver |
| | metabolizes less efficiently |
| RENAL SYSTEM | After 40 renal function decreases, By 90 lose |
| | 50% of function, Filtration and reabsorption |
| | reduced, Size and number of nephrons |
| | decrease, |
| | Bladder muscles weaken, Less able to clear |
| | drugs from system, Smaller kidneys and |
| | bladder |
| REPRODUCTIVE | Reduced testosterone level, Testes atrophy |
| SYSTEM | and soften, Decrease in sperm production, |
| (MALE) | Seminal fluid decreases and more viscous, |
| | Erections take more time, Refractory period |
| | after ejaculation may lengthen to days |
| REPRODUCTIVE | Declining estrogen and progesterone levels, |
| SYSTEM | Ovulation ceases, Introitus constricts and |
| (FEMALE) | loses elasticity, Vagina atrophies - shorter |
| | and drier, |
| | Uterus shrinks, Breasts pendulous and lose |
| | elasticity |
| NEUROLOGICAL | Neurons of central and peripheral nervous |
| SYSTEM | system degenerate, Nerve transmission slows, |
| | Hypothalamus less effective in regulating |
| | body temperature, Reduced REM sleep, |
| | decreased deep sleep, After age 50, lose 1% |
| | of neurons each year |
| MUSCULOSCELETAL | Adipose tissue increases with age, Lean body |
| SYSTEM | mass decreases, Bone mineral content |
| | diminished, Decrease in height from narrow |
| | vertebral spaces, Less resilient connective |
| | tissue, Synovial fluid more viscous, |
| | May have exaggerated curvature of spine |
| IMMUNE | Decline in immune function, Trouble |
| SYSTEM | differentiating between self and non-self - |
| | more auto-immune problems, Decreases |
| | antibody response, |
| | Fatty marrow replaced red marrow, Vitamin |
| | B12 absorption might decrease - decreased |
| | hemoglobin and hematocrit |
| ENDOCRINE | Decreased ability to tolerate stress - best |
| SYSTEM | seen in glucose metabolism, |
| | Estrogen levels decrease in women, Other |
| | hormonal decreases include testosterone, |
| | aldosterone, cortisol, progesterone |

Adapted from http://www.texashste.com/html/ger_pap1.ppt

The aging human liver appears to preserve its morphology and function relatively well. The liver appears to progressively decrease in both mass and volume. It also appears browner (a condition called “brown atrophy”), as a result of accumulation of lipofuscin (ceroid) within hepatocytes. Increases occur in the number of macrohepatocytes, and in polyploidy, especially around the terminal hepatic veins. The number of mitochondria declines, and both the rough and smooth endoplasmic recticulum diminish. The number of lysozymes increase.

The liver is the premiere metabolic organ of the body. With regard to metabolism, hepatic glycerides and cholesterol levels increase with age, at least up to age 90. On the other hand, phospholipids, aminotransferases, and serum bilirubin appear to remain normal. There are contradictory reports as to the effect of aging on albumin, serum gamma-glutamyltransferase, and hepatic alkaline phosphatase. It is worth noting that it has been shown that the content of cytochrome oxidase exhibits a progressive decline which correlates with age-associated decline in mtRNA synthesis in brain, liver, heart, lungs and skeletal muscle.

See generally Anaantharaju, Feller and Chedid, “Aging Liver: A Review,” Gerontology, 48: 343-53 (2002).

Quality of Life

Clinicians are interested, not only in simple prolongation of lifespan, but also in maintenance of a high quality of life (QOL) over as much as possible of that lifespan. QOL can be defined subjectively in terms of the subject's satisfaction with life, or objectively in terms of the subject's physical and mental ability (but not necessarily willingness) to engage in “valued activities”, such as those which are pleasurable or financially rewarding.

Flanagan has defined five domains of QOL, capturing 15 dimensions of life quality. The five domains, and their component dimensions, are physical and material well being (Material well-being and financial security; Health and personal safety)” Relations with other people (relations with spouse; Having and rearing children; Relations with parents, siblings, or other relatives; Relations with friends) Social, community, civic activities (Helping and encouraging others; Participating in local and governmental affairs), Personal development, fulfillment (Intellectual development; Understanding and planning; Occupational role career; Creativity and personal expression), and recreation (Socializing with others; Passive and observational recreational activities; Participating in active recreation). See Flanagan J C,. “A research approach to improving our quality of life.” Am Psychol 33:138-147 (1978).

“Health-related quality of life” (HRQL or HRQOL) is an individual's satisfaction or happiness with domains of life insofar as they affect or are affected by “health”.

In a preferred embodiment, a pharmaceutical agent of the present invention is able to achieve a statistically significant improvement in the expected quality of life, measured according to a commonly accepted measure of QOL, in a treatment group over a control group.

While there is general acceptance of the notion that QOL is important, quantifying QOL is not especially straightforward. Also, QOL can only be measured in humans. Measurements of QOL can be objective (e.g., employment status, marital status, home ownership) or subjective (the subject's opinion of his or her life), or some combination of the two.

A simple approach to measuring subjective QOL is to simply have the subjects rate their overall quality of life on a scale, e.g., of 7 points. One can also use more elaborate measure, such as the Older Adult Health and Mood Questionaire (a 22 item test for assessing depression). Objective QOL can be measured by, e.g., an activities checklist.

There is a relationship between QOL assessment and so-called ADL or IADL measures, which assess the need for assistance.

The Katz Index of Independence in Activities of Daily Living (Katz ADL) measures adequacy of independent performance of bathing, dressing, toileting, transferring, continence, and feeding. See Katz, S., “Assessing Self-Maintenance: Activities of Daily Living, Mobility and Instrumental Activities of Daily Living, Journal of the American Geriatrics Society, 31(12); 72L-726 (1983); Katz S., Down, T. D., Cash, H. R. et al. Progress in the Development of the Index of ADL. Gerontologist,l0: 20-30 (1970).

Performance of a more sophisticated nature is measured by the “Instrumental Activities of Daily Living” (IADL) scale. This inquires into ability to independently use the telephone, shop, prepare food, carry out housekeeping, do laundry, travel locally, take medication and handle finances. See Lawtori, M P and Brody, E M, Gerontologist, 9:179-86 (1969).

The 36 question Medical Outcomes Study Short Form (SF-36) (Medical Outcomes Trust, Inc., 20 Park Plaza, Suite 1014, Boston, Mass. 02116) assesses eight health concepts: 1) limitations in physical activities because of health problems; 2) limitations in social activities because of physical or emotional problems; 3) limitations in usual role activities because of physical health problems; 4) bodily pain; 5) general mental health (psychological distress and well-being); 6) limitations in usual role activities because of emotional problems; 7) vitality (energy and fatigue); and 8) general health perceptions.

A low score on an ADL, IADL or SF-36 test is likely to be associated with a low QOL, but a high score does not guarantee a high QOL because these tests do not explore performance of “valued activities”, only of more basic activities. Nonetheless, these tests can be considered commonly accepted measures of QOL for the purpose of this invention.

Age-Related Diseases

Age-related (senescent) diseases include certain cancers, atherosclerosis, diabetes (type 2), osteoporosis, hypertension, depression, Alzheimer's, Parkinson's, glaucoma, certain immune system defects, kidney failure, and liver steatosis. In general, they are diseases for which the relative risk (comparing a subpopulation over age 55 to a suitably matched population under age 55) is at least 1.1.

Preferably, the agents of the present invention protect against one or more age-related diseases for at least a subpopulation of mature (post-puberty) adult subjects.

Diabetes

Type II diabetes is of particular interest. A deficiency of insulin in the body results in diabetes mellitus, which affects about 18 million individuals in the United States. It is characterized by a high blood glucose (sugar) level and glucose spilling into the urine due to a deficiency of insulin. As more glucose concentrates in the urine, more water is excreted, resulting in extreme thirst, rapid weight loss, drowsiness, fatigue, and possibly dehydration. Because the cells of the diabetic cannot use glucose for fuel, the body uses stored protein and fat for energy, which leads to a buildup of acid (acidosis) in the blood. If this condition is prolonged, the person can fall into a diabetic coma, characterized by deep labored breathing and fruity-odored breath.

There are two types of diabetes mellitus, Type I and Type II. Type II diabetes is the predominant form found in the Western world; fewer than 8% of diabetic Americans have the type I disease.

Type I diabetes. In Type I diabetes, formerly called juvenile-onset or insulin-dependent diabetes mellitus, the pancreas cannot produce insulin. People with Type I diabetes must have daily insulin injections. But they need to avoid taking too much insulin because that can lead to insulin shock, which begins with a mild hunger. This is quickly followed by sweating, shallow breathing, dizziness, palpitations, trembling, and mental confusion. As the blood sugar falls, the body tries to compensate by breaking down fat and protein to make more sugar. Eventually, low blood sugar leads to a decrease in the sugar supply to the brain, resulting in a loss of consciousness. Eating a sugary food can prevent insulin shock until appropriate medical measures can be taken.

Type I diabetics are often characterized by their low or absent levels of circulating endogenous insulin, i.e., hypoinsulinemia (1). Islet cell antibodies causing damage to the pancreas are frequently present at diagnosis. Injection of exogenous insulin is required to prevent ketosis and sustain life.

Type II diabetes. Type II diabetes, formerly called adult-onset or non-insulin-dependent diabetes mellitus (NIDDM), can occur at any age. The pancreas can produce insulin, but the cells do not respond to it.

Type II diabetes is a metabolic disorder that affects approximately 17 million Americans. It is estimated that another 10 million individuals are “prone” to becoming diabetic. These vulnerable individuals can become- resistant to insulin, a pancreatic hormone that signals glucose (blood sugar) uptake by fat and muscle. In order to maintain normal glucose levels, the islet cells of the pancreas produce more insulin, resulting in a condition called hyperinsulinemia. When the pancreas can no longer produce enough insulin to compensate for the insulin resistance, and thereby maintain normal glucose levels, hyperglycemia (elevated blood glucose) results, and type II diabetes is diagnosed.

Early Type II diabetics are often characterized by hyperinsulinemia and resistance to insulin. Late Type II diabetics may be normoinsulinemic or hypoinsulinemic. Type II diabetics are usually not insulin dependent or prone to ketosis under normal circumstances.

Little is known about the disease progression from the normoinsulinemic state to the hyperinsulinemic state, and from the hyperinsulinemic state to the Type II diabetic state.

As stated above, type II diabetes is a metabolic disorder that is characterized by insulin resistance and impaired glucose-stimulated insulin secretion (2, 3, 4). However, Type II diabetes and atherosclerotic disease are viewed as consequences of having the insulin resistance syndrome (IRS) for many years (5). The current theory of the pathogenesis of Type II diabetes is often referred to as the “insulin resistance/islet cell exhaustion” theory. According to this theory, a condition causing insulin resistance compels the pancreatic islet cells to hypersecrete insulin in order to maintain glucose homeostasis. However, after many years of hypersecretion, the islet cells eventually fail and the symptoms of clinical diabetes are manifested. Therefore, this theory implies that, at some point, peripheral hyperinsulinemia will be an antecedent of Type II diabetes. Peripheral hyperinsulinemia can be viewed as the difference between what is produced by the beta cell minus that which is taken up by the liver. Therefore, peripheral hyperinsulinemia can be caused by increased β cell production, decreased hepatic uptake or some combination of both It is also important to note that it is not possible to determine the origin of insulin resistance once it is established since the onset of peripheral hyperinsulinemia leads to a condition of global insulin resistance.

Multiple environmental and genetic factors are involved in the development of insulin resistance, hyperinsulinemia and type II diabetes. An important risk factor for the development of insulin resistance, hyperinsulinemia and type II diabetes is obesity, particularly visceral obesity (6, 7, 8). Type II diabetes exists world-wide, but in developed societies, the prevalence has risen as the average age of the population increases and the average individual becomes more obese.

Role of the Liver in the Development of Diabetes

Insulin stimulates the liver to store glucose in the form of glycogen. A large fraction of glucose absorbed from the small intestine is immediately taken up by hepatocytes, which convert it into the storage polymer glycogen. Hepatic uptake of insulin is a function of the number and efficiency of the liver's insulin receptors, and the factors which affect them are not well understood.

In the liver, insulin activates the enzyme hexokinase, which phosphorylates glucose, trapping it within the cell. Insulin also activates several of the enzymes that are directly involved in glycogen synthesis, including phosphofructokinase and glycogen synthase. However, insulin also acts to inhibit the activity of glucose-6-phosphatase.

When the liver is saturated with glycogen, any additional glucose taken up by hepatocytes is shunted into pathways leading to synthesis of fatty acids, which are exported from the liver as lipoproteins. The lipoproteins are ripped apart in the circulation, providing free fatty acids for use in other tissues, including adipocytes, which use them to synthesize triglyceride.

In the absence of insulin, glycogen synthesis in the liver ceases and enzymes responsible for breakdown of glycogen become active.

As noted above, peripheral hyperinsulinemia can be viewed as the difference between what insulin is produced by the β cell minus that which is taken up by the liver. Therefore, peripheral hyperinsulinemia can be caused by increased β cell production, decreased hepatic uptake or some combination of both.

Effect of Diabetes on the Liver

Diabetes is associated with nonalcoholic steatohepatitis (NASH), also known as nonalcoholic fatty liver disease (NAFLD). In NASH, fat builds up in the liver and eventually causes scar tissue (cirrhosis of the liver).

Non-alcoholic fatty liver disease (NAFLD) is now recognized as one of the most common causes of liver disease and is estimated to affect 10 to 24% of the general population. The hither prevalence of NAFLD in persons with obesity, hyperinsulinemia or type-II diabetes suggests that diet and insulin resistance may play a pivotal role in the development of this syndrome. NAFLD is a clinicopathologic syndrome with a wide spectrum of liver damage ranging from simple steatosis to steatohepatitis (NASH) to advanced fibrosis and cirrhosis. Hepatic steatosis is caused by lipid accumulation within hepatocytes and is a relatively benign condition. However steatosis combined with necro-inflammatory activity may progress to end-stage liver disease. It appears that the disease progression requires cellular injury and inflammation in a steatotic environment. While the cause of the injury is not understood, it is clear that hepatic apoptosis is a prominent feature of non-alcoholic steatosis as well as other liver diseases. See generally Alba, L. M., Lindor, K. (2003) Review article: Non-alcoholic fatty liver disease., Aliment Pharmacol. Them. 17:977-986; Ludwig, J., Viggiano, T. R., McGill, D. B., Oh, B. J. (1980) Nonalcoholic steatohepatitis: Mayo Clinic experiences with a hitherto unnamed disease. Mayo Clin. Proc. 55:434-438; Chitturi, S., Abeygunasekera, S., Farrel, G. C., Holmes-Walker, J., Hui, J. M., Fung, C., Karim, R., Lin, R., Samarasinghe, D., Liddle, C., Weltman, M., George, J. (2002) NASH and insulin resistance: Insulin hypersecretion and specific association with the insulin resistance syndrome. Hepatology 35:373-379; Feldstein, A. E., Canbay, A., Angulo, P., Taniai, M., Burgart, L. J., Lindor, K. D., Gores, G. J. (2003) Hepatocyte apoptosis and fas expression are prominent features of human nonalcoholic steatohepatitis. Gastroenterology 125:437-443; Higuch, H., Gores, G. J. (2003) Mechanisms of liver injury: an overview. Curr. Mol. Med. 3:483-490.

Drugs used for the treatment of diabetes, such as Rezulin (troglitazone), can cause liver damage.

Diseases Characterized by Accelerated Aging

Several human diseases display some features of accelerated aging. These include Werner's syndrome (classic early-onset progeria), Hutchinson-Gilford syndrome (adult progeria), and Down's syndrome (trisomy 21). Troen, Biology of Aging, Mt. Sinai J. Med., 70(1): 3 (January 2003). Thus, the present invention may be useful in the treatment (curative or ameliorative) of individuals with these diseases.

Direct and Indirect Utility of Identified Nucleic Acid Sequences and Related Molecules

The mouse or human genes may be used directly. For diagnostic or screening purposes, they (or specific binding fragments thereof) may be labeled and used as hybridization probes. For therapeutic purposes, they (or specific binding fragments thereof) may be used as antisense reagents to inhibit the expression of the corresponding gene, or of a sufficiently homologous gene of another species.

If the database DNA appears to be a full-length cDNA or gDNA, that is, that it encodes an entire, functional, naturally occurring protein, then it may be used in the expression of that protein. Likewise, if the corresponding human gene is known in full-length, it may be used to express the human protein Such expression may be in cell culture, with the protein subsequently isolated and administered exogenously to subjects who would benefit therefrom, or in vivo, i.e., administration by gene therapy. Naturally, any DNA encoding the same protein may be used for the same purpose, or a DNA which encodes a fragment or a mutant of that naturally occurring protein which retains the desired activity may be used for the purpose of producing the active fragment or mutant. The encoded protein of coarse has utility therapeutically and, in labeled or immobilized form, diagnostically.

The genes may also be used indirectly, that is, to identify other useful DNAs, proteins, or other molecules. We have attempted to determine whether the mouse genes disclosed herein have significant similarity to any known human DNA, and whether, in any of the six possible combinations of reference frame and strand, they encode a protein similar to a known human protein. If so, then it follows that the known human protein, and DNAs encoding that protein, may be used in a similar manner. In addition, if the known human protein is known to have additional homologues, then those homologous proteins, and DNAs encoding them, may be used in a similar manner.

There thus are several ways that a human protein homologue of interest can be identified by database searching, including:
1) a DNA→DNA (BlastN) search for human database DNAs closely related to the mouse gene identifies a known human gene, and the sequence of the human-protein is deduced by the Genetic Code;
2) a DNA→Protein (BlastX) search for human database proteins closely related to the translated DNA of the mouse gene identifies a known human protein; and
3) the sequence of the mouse protein is known or is deduced by the Genetic Code, and a Protein→Protein (BlastP) search for closely related database proteins identifies a known human protein.

Once a known human gene is identified, it may be used in further BlastN or BlastX searches to identify other human genes or proteins. Once a known human protein is identified, it may be used in further BlastP searches to identify other human proteins. Searches may also take cognizance, intermediately, of known genes and proteins other than mouse or human ones, e.g., use the mouse sequence to identify a known rat sequence and then the rat sequence to identify a human one.

If we have identified a mouse gene (gDNA or CDNA), and it encodes a mouse protein which appears similar to a human protein, then that human protein may be used (especially in humans) for purposes analogous to the proposed use of the mouse protein in mice. Moreover, a specific binding fragment of an appropriate strand of the corresponding human gene (gDNA or cDNA) could be labeled and used as a hybridization probe (especially against samples of human mRNA or cDNA).

In determining whether the disclosed genes (GDNA or cDNA) have significant similarities-to known DNAs (and their translated AA sequences to known proteins) one would generally use the disclosed gene as a query sequence in a search of a sequence database. The results of several such searches are set forth in the Examples. Such results are dependent, to some degree, on the search parameters. Preferred parameters are set forth in Example 1. The results are also dependent on the content of the database. While the raw similarity score of a particular target (database) sequence will not vary with content (as long as it remains in the database), its informational value (in bits), expected value, and relative ranking can change. Generally speaking, the changes are small.

It will be appreciated that the nucleic acid and protein databases keep growing. Hence a later search may identify high scoring target sequences which were not uncovered by an earlier search because the target sequences were not previously part of a database.

Hence, in a preferred embodiment, the cognate DNAs and proteins include not only those set forth in the examples, but those which would have been highly ranked (top ten, more preferably top three, even more preferably top two, most preferably the top one) in a search run with the same parameters on the date of filing of this application.

If the mouse or human database DNA appears to be a partial DNA (that is, partial relative to a cDNA or gDUA encoding the whole naturally occurring protein), it may be used as a hybridization probe to isolate the full-length DNA. If the partial DNA encodes a biologically functional fragment of the cognate protein, it may be used in a manner similar to the full length DNA, i.e., to produce the functional fragment.

If we have indicated that an antagonist of a protein or other molecule is useful, then such an antagonist may be obtained by preparing a combinatorial library, as described below, of potential antagonists, and screening the library members for binding to the protein or other molecule in question. The binding members may then be further screened for the ability to antagonize the biological activity of the target. The antagonists may be used therapeutically, or, in suitably labeled or immobilized form, diagnostically.

If the mouse or human database DNA is related to a known protein, then substances known to interact with that protein (e.g., agonists, antagonists, substrates, receptors, second messengers, regulators, and so forth), and binding molecules which bind them, are also of utility. Such binding molecules can likewise be identified by screening a combinatorial library.

Isolation of Full Length DNAs Using Partial DNAs as Probes

If it is determined that a DNA of the present invention is a partial DNA, and the cognate full length DNA is not listed in a sequence database, the available DNA may be used as a hybridization probe to isolate the full-length DNA from a suitable DNA library (CDNA or gDNA).

Stringent hybridization conditions are appropriate, that is, conditions in which the hybridization temperature is 5-10 deg. C. below the Tm of the DNA as a perfect duplex.

Identification and Isolation of Homologous Genes Using a DNA Probe

It may be that the sequence databases available do not include the sequence of any homologous gene (gDNA or CDNA), or at least of the homologous gene for a species of interest. However, given the DNAs set forth above, one may readily obtain the homologous gene.

The possession of one DNA (the “starting DNA”) greatly facilitates the isolation of homologous DNAs. If only a partial DNA is known, this partial DNA may first be used as a probe to isolate the corresponding full length DNA for the same species, and that the latter may be used as the starting DNA in the search for homologous DNAs.

The starting DNA, or a fragment thereof, is used as a hybridization probe to screen a cDNA or genomic DNA library for clones containing inserts which encode either the entire homologous protein, or a recognizable fragment thereof. The minimum length of the hybridization probe is dictated by the need for specificity. If the size of the library in bases is L, and the GC content is 50%, then the probe should have a length of at least 1, where L=4¹. This will yield, on average, a single perfect match in random DNA of L bases. The human cDNA library is about 10⁸ bases and the human genomic DNA library is about 10¹⁰ bases.

The library is preferably derived from an organism which is known, on biochemical evidence, to produce a homologous protein, and more preferably from the genomic DNA or mRNA of cells of that organism which are likely to be relatively high producers of that protein. A cDNA library (which is derived from an mRNA library) is especially preferred.

If the organism in question is known to have substantially different codon preferences from that of the organism whose relevant cDNA or genomic DNA is known, a synthetic hybridization probe may be used which encodes the same amino acid sequence but whose codon utilization is more similar to that of the DNA of the target organism. Alternatively, the synthetic probe may employ inosine as a substitute for those bases which are most likely to be divergent, or the probe may be a mixed probe which mixes the codons for the source DNA with the preferred codons (encoding the same amino acid) for the target organism.

By routine methods, the Tm of a perfect duplex of starting DNA is determined. One may then select a hybridization temperature which is sufficiently lower than the perfect duplex Tm to allow hybridization of the starting DNA (or other probe) to a target DNA which is divergent from the starting DNA. A 1% sequence divergence typically lowers the Tm of a duplex by 1-2° C., and the DNAs encoding homologous proteins of different species typically have sequence identities of around 50-80%. Preferably, the library is screened under conditions where the temperature is at least 20° C., more preferably at least 50° C., below the perfect duplex Tm Since salt reduces the Tm, one ordinarily would carry out the search for DNAs encoding highly homologous proteins under relatively low salt hybridization conditions, e.g., <1M NaCl. The higher the salt concentration, and/or the lower the temperature, the greater the sequence divergence which is tolerated.

For the use of probes to identify homologous genes in other species, see, e.g., Schwinn, et al., J. Biol. Chem., 265:8183-89 (1990) (hamster 67-bp CDNA probe vs. human leukocyte genomic library; human 0.32 kb DNA probe vs. bovine brain cDNA library, both with hybridization at 42° C. in 6×SSC); Jenkins et al., J. Biol. Chem., 265:19624-31 (1990) (Chicken 770-bp cDNA probe vs. human genomic libraries; hybridization at 40° C. in 50% formamide and 5×SSC); Murata et al., J. Exp. Med., 175:341-51 (1992) (1.2-kb mouse cDNA probe v. human eosinophil cDNA library; hybridization at 65° C. in 6×SSC); Guyer et al., J. Biol. Chem., 265:17307-17 (1990) (2.95-kb human genomic DNA probe vs. porcine genomic DNA library; hybridization at 42° C. in 5×SSC). The conditions set forth in these articles may each be considered suitable for the purpose of isolating homologous genes.

Corresponding (Homologous) Proteins and DNAs

In the case of a gene chip, the manufacturer of the gene chip determines which DNA to place at each position on the chip. This DNA may correspond in sequence to a genomic DNA, a cDNA, or a fragment of genomic or cDNA, and may be natural, synthetic or partially natural and partially synthetic in origin. The manufacturer of the gene chip will normally identify the DNA for a mouse gene chip as corresponding to a particular mouse gene, in which case it will be assumed that the alignments of chip DNA to mouse gene satisfies the correspondence (homology) criteria of the invention.

Usually, the gene chip manufacturer will provide a sequence database accession number for the mouse DNA. If so, to identify the corresponding mouse protein, we will first inspect the database record for that mouse DNA. Often, the mouse protein accession number will appear in that record or in a linked record. If it doesn't, the corresponding mouse protein can be identified by performing a BlastX search on a mouse protein database with the mouse database DNA sequence as the query sequence. Even if the protein sequence is not in the database, if the DNA sequence comprises a full-length coding sequence, the corresponding protein can be identified by translating the coding sequence in accordance with the Genetic Code.

A human protein can be said to be identifiable as corresponding (homologous) to a gene chip DNA if it is identified as corresponding (homologous ) to the mouse gene (gDNA or cDNA, whole or partial) identified by the gene chip manufacturer as corresponding (homologous)to that gene chip DNA.

In turn, it is identifiable as corresponding (homologous) to said identified mouse gene, if
(1) it can be aligned by BlastX directly to that mouse gene, and/or
(2) it is encoded by a human gene, or can be aligned to a human gene by BlastX, which in turn can be aligned by BlastN to said mouse gene and/or
(3) it can be aligned by BlastP to a mouse protein, the latter being encoded by said mouse gene, or aligned to said mouse gene BlastX,
   where any alignment by BlastN, BlastP or BlastX is in accordance with the default parameters set forth below, and the expected value (E) of each alignment (the probability that such an alignment would have occurred by chance alone) is less than e-10. (Note that because this is a negative exponent, a value such as e-50 is less than e-10.)

A human gene is corresponding (homologous) to a mouse gene chip DNA, and hence to said identified mouse gene (or cDNA) and protein, if it encodes a corresponding (homologous) human protein as defined above, or it can be aligned by BlastN to said mouse gene.

Desirably, two or all three of these conditions (1)-(3) are satisfied for the corresponding (homologous) human genes and proteins.

Preferably, for at least one of conditions (1)-(3), the E value is less than e-50, more preferably less than e-60, still more preferably less than e-70, even more preferably less than e-80, considerably more preferably less than e-90, and most preferably less than e-100. Desirably, it is true for two or even all three of these conditions.

In constructing Master table 1, we generally used a BlastX (mouse gene vs. human protein) alignment E value cutoff of e-50. However, if there were no human proteins with that good an alignment to the mouse DNA in question, or if there were other reasons for including a particular human protein (e.g., a known functionality supportive of the observed differential cognate mouse protein expression), them a human protein with a score worse (i.e., higher) than e-50 may appear in Master Table 1.

BlastN and BlastX report very low expected values as “0.0”. This does not truly mean that the expected value is exactly zero (since any alignment could occur by chance), but merely that it is so infinitesimal that it is not reported. The documentation does not state the cutoff value, alignments with explicit E values as low as e-178 (624 bits) have been reported as such, while a score of 636 bits was reported as “0.0”.

If the manufacturer of the gene chip identifies the gene chip DNA as corresponding to an EST, or other DNA which is not a full-length mouse gene or cDNA, a longer (possibly full length) mouse gene or cDNA may be identified by a BlastN search of the mouse DNA database. Alternatively, the identified DNA may be used to conduct a BlastN search of a human DNA database, or a BlastX search of a mouse or human protein database.

Thus, more generally, a human protein can be said to be identifiable as corresponding (homologous) to a gene chip DNA, or to a DNA identified by the manufacturer as corresponding to that gene chip DNA, if
(1′) it can be aligned directly to the gene chip or corresponding manufacturer identified DNA by BlastX. and/or
(2′) it can be aligned to a human gene/cDNA by BlastX, whose genomic DNA (gDNA) or cDNA (DNA complementary to messenger RNA) in turn can be aligned to the gene chip or corresponding manufacturer identified DNA by BlastN, and/or
(3′) it can be aligned to a mouse gene/cDNA by BlastX, whose gDNA or cDNA in turn can be aligned to the gene chip or corresponding manufacturer identified DNA by BlastN, and/or
(4′) it can be aligned to a mouse protein by BlastP, which in turn can be aligned to the gene chip or corresponding manufacturer identified DNA by BlastX, and/or
(5′) it can be aligned to a mouse protein by BlastP, which in turn can be aligned to a mouse gene/cDNA by BlastX, whose gDNA or cDNA can in turn be aligned to the gene chip or corresponding manufacturer identified DNA by BlastN;
   where any alignment by BlastN, BlastP, or BlastX is in accordance with the default parameters set forth below, and the expected value (E) of each alignment (the probability that such an alignment would have occurred by chance alone) is less than e-10. (Note that because this is a negative exponent, a value such as e-50 is less than e-10.) Preferably, two, three, four or all five of conditions (1′)-(5′) are satisfied.

Preferably, for at least one of conditions (1′)-(5′), for at least the final alignment (i.e., vs. the human protein), the E value is less than e-50, more preferably less than e-60, still more preferably less than e-70, even more preferably less than e-80, considerably more preferably less than e-90, and most preferably less than e-100.

Desirably, one or more of these standards of preference are met for two, three, four or all five of conditions (1′)-(5′). In particular, for those conditions in which the gene chip or corresponding manufacturer identified DNA is indirectly connected to the human protein by virtue of two or more successive alignments, the E value is preferably, so limited for all of said alignments in the connecting chain.

A human gene corresponds (is homologous) to a gene chip DNA or manufacturer identified corresponding DNA if it encodes a corresponding (homologous) human protein as defined above, or if it can be aligned either directly to that DNA, or indirectly through a mouse gene which can be aligned to said DNA, according to the conditions set forth above.

Master table 1 assembles a list of human protein corresponding (homologous) to each of the mouse DNAs/proteins identified as related to the chip DNA. These human proteins form a set and can be given a percentile rank, with respect to E value, within that set. The human proteins of the present invention preferably are those scorers with a percentile rank of at least 50%, more preferably at least 60%, still more preferably at least 70%, even more preferably at least 80%, and most preferably at least 90%.

For each mouse gene in Master Table 1, there is a particular human protein which provides the best alignment match as measured by BlastX, i.e., the human protein with the best score (lowest e-value). These human proteins form a subset of the set above and can be given a percentile rank within that subset, e.g., the human proteins with scores in the top 10% of that subset have a percentile rank of 90% or higher.

The human proteins of the present invention preferably are those best scorer subset proteins with a percentile rank within the subset of at least 50%, more preferably at least 60%, still more preferably at least 70%, even more preferably at least 80%, and most preferably at least 90%.

BlastN and BlastX report very low expected values as “0.0”. This does not truly mean that the expected value is exactly zero (since any alignment could occur by chance), but merely that it is so infinitesimal that it is not reported. The documentation does not state the cutoff value, but alignments with explicit E values as low as e-178 (624 bits) have been reported as nonzero values, while a score of 636 bits was reported as “0.0”.

Functionally homologous human proteins are also of interest. A human protein may be said to be functionally homologous to the mouse gene if the human protein has at least one biological activity in common with the mouse protein encoded by said mouse gene.

The human proteins of interest also include those that are substantially and/or conservatively identical (as defined below) to the homologous and/or functionally homologous human proteins defined above.

Degree of Differential Expression

The degree of differential expression may be expressed as the ratio of the higher expression level to the lower expression level. Preferably, this is at least 2-fold, and more preferably, it is higher, such as at least 3-fold, at least 4-fold, at least 5-fold, at least 6-fold, at least 7-fold, at least 8-fold, at least 9-fold, or at least 10-fold.

Most preferably, the human protein of interest corresponds to a mouse gene for which the degree of differential expression places it among the top 10% of the mouse genes in the appropriate subtable.

Relevance of Favorable and Unfavorable Genes

If a gene is down-regulated in more favored mammals, or up-regulated in less favored mammals, (i.e., an “unfavorable gene”) then several utilities are apparent.

First, the complementary strand of the gene, or a portion thereof, may be used in labeled form as a hybridization probe to detect messenger RNA and thereby monitor the level of expression of the gene in a subject. Elevated levels are indicative of progression, or propensity to progression, to a less favored state, and clinicians may take appropriate preventative, curative or ameliorative action.

Secondly, the messenger RNA product (or equivalent cDNA), the protein product, or a binding molecule specific for that product (e.g., an antibody which binds the product), or a downstream product which mediates the activity (e.g., a signaling intermediate) or a binding molecule (e.g., an antibody) therefor, may be used, preferably in labeled or immobilized form, as an assay reagent in an assay for said nucleic acid product, protein product, or downstream product (e.g., a signaling intermediate). Again, elevated levels are indicative of a present or future problem.

Thirdly, an agent which down-regulates expression of the gene may be used to reduce levels of the corresponding protein and thereby inhibit further damage. This agent could inhibit transcription of the gene in the subject, or translation of the corresponding messenger RNA. Possible inhibitors of transcription and translation include antisense molecules and repressor molecules. The agent could also inhibit a post-translational modification (e.g., glycosylation, phosphorylation, cleavage, GPI attachment) required for activity, or post-translationally modify the protein so as to inactivate it. Or it could be an agent which down- or up-regulated a positive or negative regulatory gene, respectively.

Fourthly, an agent which is an antagonist of the messenger RNA product or protein product of the gene, or of a downstream product through which its activity is manifested (e.g., a signaling intermediate), may be used to inhibit its activity.

This antagonist could be an antibody, a peptide, a peptoid, a nucleic acid, a peptide nucleic acid (PNA) oligomer, a small organic molecule of a kind for which a combinatorial library exists (e.g., a benzodiazepine), etc. An antagonist is simply a binding molecule which, by binding, reduces or abolishes the undesired activity of its target. The antagonist, if not an oligomeric molecule, is preferably less than 1000 daltons, more preferably less than 500 daltons.

Fifthly, an agent which degrades, or abets the degradation of, that messenger RNA, its protein product or a downstream product which mediates its activity (e.g., a signaling intermediate), may be used to curb the effective period of activity of the protein.

If a gene is up-regulated in more favored mammals, or down-regulated in less favored animals then the utilities are converse to those stated above.

First, the complementary strand of the gene, or a portion thereof, may be used in labeled form as a hybridization probe to detect messenger RNA and thereby monitor the level of expression of the gene in a subject. Depressed levels are indicative of damage, or possibly of a propensity to damage, and clinicians may take appropriate preventative, curative or ameliorative action.

Secondly, the messenger RNA product, the equivalent cDNA, protein product, or a binding molecule specific for those products, or a downstream product, or a signaling intermediate, or a binding molecule therefor, may be used, preferably in labeled or immobilized form, as an assay reagent in an assay for said protein product or downstream product. Again, depressed levels are indicative of a present or future problem.

Thirdly, an agent which up-regulates expression of the gene may be used to increase levels of the corresponding protein and thereby inhibit further progression to a less favored state. By way of example, it could be a vector which carries a copy of the gene, but which expresses the gene at higher levels than does the endogenous expression system. Or it could be an agent which up- or down-regulates a positive or negative regulatory gene.

Fourthly, an agent which is an agonist of the protein product of the gene, or of a downstream product through which its activity (of inhibition of progression to a less favored state) is manifested, or of a signaling intermediate may be used to foster its activity.

Fifthly, an agent which inhibits the degradation of that protein product or of a downstream product or of a signaling intermediate may be used to increase the effective period of activity of the protein.

Mutant Proteins

The present invention also contemplates mutant proteins (peptides) which are substantially identical (as defined below) to the parental protein (peptide). In general, the fewer the mutations, the more likely the mutant protein is to retain the activity of the parental protein. The effect of mutations is usually (but not always) additive. Certain individual mutations are more likely to be tolerated than others.

A protein is more likely to tolerate a mutation which
(a) is a substitution rather than an insertion or deletion;
(b) is am insertion or deletion at the terminus, rather than internally, or, if internal, is at a domain boundary, or a loop or turn, rather than in an alpha helix or beta strand;
(c) affects a surface residue rather than an interior residue;
(d) affects a part of the molecule distal to the binding site;
(e) is a substitution of one amino acid for another of similar size, charge, and/or hydrophobicity, and does not destroy a disulfide bond or other crosslink; and
(f) is at a site which is subject to substantial variation among a family of homologous proteins to which the protein of interest belongs.
   These considerations can be used to design functional mutants.
   Surface vs. Interior Residues

Charged amino acid residues almost always lie on the surface of the protein. For uncharged residues, there is less certainty, but in general, hydrophilic residues are partitioned to the surface and hydrophobic residues to the interior. Of course, for a membrane protein, the membrane-spanning segments are likely to be rich in hydrophobic residues.

Surface residues may be identified experimentally by various labeling techniques, or by 3-D structure mapping techniques like X-ray diffraction and NMR. A 3-D model of a homologous protein can be helpful.

Binding Site Residues

Residues forming the binding site may be identified by (1) comparing the effects of labeling the surface residues before and after complexing the protein to its target, (2) labeling the binding site directly with affinity ligands, (3) fragmenting the protein and testing the fragments for binding activity, and (4) systematic mutagenesis (e.g., alanine-scanning mutagenesis) to determine which mutants destroy binding. If the binding site of a homologous protein is known, the binding site may be postulated by analogy.

Protein libraries may be constructed and screened that a large family (e.g., 10⁸) of related mutants may be evaluated simultaneously. Hence, the mutations are preferably conservative modifications as defined below.

“Substantially Identical”

A mutant protein (peptide) is substantially identical to a reference protein (peptide) if (a) it has at least 10% of a specific binding activity or a non-nutritional biological activity of the reference protein, and (b) is at least 50% identical in amino acid sequence to the reference protein (peptide). It is “substantially structurally identical” if condition (b) applies, regardless of (a)

Percentage amino acid identity is determined by aligning the mutant and reference sequences according to a rigorous dynamic programming algorithm which globally aligns their sequences to maximize their similarity, the similarity being scored as the sum of scores for each aligned pair according to an unbiased PAM250 matrix, and a penalty for each internal gap of −12 for the first null of the gap and −4 for each additional null of the same gap. The percentage identity is the number of matches expressed as a percentage of the adjusted (i.e., counting inserted nulls) length of the reference sequence.

A mutant DNA sequence is substantially identical to a reference DNA sequence if they are structural sequences, and encoding mutant and reference proteins which are substantially identical as described above.

If instead they are regulatory sequences, they are substantially identical if the mutant sequence has at least 10% of the regulatory activity of the reference sequence, and is at least 50% identical in nucleotide sequence to the reference sequence. Percentage identity is determined as for proteins except that matches are scored +5, mismatches −4, the gap open penalty is −12, and the gap extension penalty (per additional null) is −4.

More preferably, the sequence is not merely substantially identical, but rather is at least 51%, 66%, 75%, 80%, 85%, 90%, 95%, 96%, 97%, 98% or 99% identical in sequence to the reference sequence.

DNA sequences may also be considered “substantially identical” if they hybridize to each other under stringent conditions, i.e., conditions at which the Tm of the heteroduplex of the one strand of the mutant DNA and the more complementary strand of the reference DNA is not in excess of 10° C. less than the Tm of the reference DNA homoduplex. Typically this will correspond to a percentage identity of 85-90%.

“Conservative Modifications”

“Conservative modifications” are defined as
(a) conservative substitutions of amino acids as hereafter defined; or
(b) single or multiple insertions (extension) or deletions (truncation) of amino acids at the termini.

Conservative modifications are preferred to other modifications. Conservative substitutions are preferred to other conservative modifications

“Semi-Conservative Modifications” are modifications which are not conservative, but which are (a) semi-conservative substitutions as hereafter defined; or (b) single or multiple insertions or deletions internally, but at interdomain boundaries, in loops or in other segments of relatively high mobility. Semi-conservative modifications are preferred to nonconservative modifications. Semi-conservative substitutions are preferred to other semi-conservative modifications.

Non-conservative substitutions are preferred to other non-conservative modifications.

The term “conservative” is used here in an a priori sense, i.e., modifications which would be expected to preserve 3D structure and activity, based on analysis of the naturally occurring families of homologous proteins and of past experience with the effects of deliberate mutagenesis, rather than post facto, a modification already known to conserve activity. Of course, a modification which is conservative a priori may, and usually is, also conservative post facto.

Preferably, except at the termini, no more than about five amino acids are inserted or deleted at a particular locus, and the modifications are outside regions known to contain binding sites important to activity.

Preferably, insertions or deletions are limited to the termini.

A conservative substitution is a substitution of one amino acid for another of the same exchange group, the exchange groups being defined as follows
I Gly, Pro, Ser, Ala (Cys) (and any nonbiogenic, neutral amino acid with a hydrophobicity not exceeding that of the aforementioned a.a.'s)
II Arg, Lys, His (and any nonbiogenic, positively-charged amino acids)
III Asp, Glu, Asn, Gln (and any nonbiogenic negatively-charged amino acids)
IV Leu, Ile, Met, Val (Cys) (and any nonbiogenic, aliphatic, neutral amino acid with a hydrophobicity too high for I above)
V Phe, Trp, Tyr (and any nonbiogenic, aromatic neutral amino acid with a hydrophobicity too high for I above).

Note that Cys belongs to both I and IV.

Residues Pro, Gly and Cys have special conformational roles. Cys participates in formation of disulfide bonds. Gly imparts flexibility to the chain. Pro imparts rigidity to the chain and disrupts a helices. These residues may be essential in certain regions of the polypeptide, but substitutable elsewhere.

One, two or three conservative substitutions are more likely to be tolerated than a larger number.

“Semi-conservative substitutions” are defined herein as being substitutions within supergroup I/II/III or within supergroup IV/V, but not within a single one of groups I-V. They also include replacement of any other amino acid with alanine. If a substitution is not conservative, it preferably is semi-conservative.

“Non-conservative substitutions” are substitutions which are not “conservative” or “semi-conservative”.

“Highly conservative substitutions” are a subset of conservative substitutions, and are exchanges of amino acids within the groups Phe/Tyr/Trp, Met/Leu/Ile/Val, His/Arg/Lys, Asp/Glu and Ser/Thr/Ala. They are more likely to be tolerated than other conservative substitutions. Again, the smaller the number of substitutions, the more likely they are to be tolerated.

“Conservatively Identical”

A protein (peptide) is conservatively identical to a reference protein (peptide) it differs from the latter, if at all, solely by conservative modifications, the protein (peptide) remaining at least seven amino acids long if the reference protein (peptide) was at least seven amino acids long.

A protein is at least semi-conservatively identical to a reference protein (peptide) if it differs from the latter, if at all, solely by semi-conservative or conservative modifications.

A protein (peptide) is nearly conservatively identical to a reference protein (peptide) if it differs from the latter, if at all, solely by one or more conservative modifications and/or a single nonconservative substitution.

It is highly conservatively identical if it differs, if at all, solely by highly conservative substitutions. Highly conservatively identical proteins are preferred to those merely conservatively identical. An absolutely identical protein is even more preferred.

The core sequence of a reference protein (peptide) is the largest single fragment which retains at least 10% of a particular specific binding activity, if one is specified, or otherwise of at least one specific binding activity of the referent. If the referent has more than one specific binding activity, it may have more than one core sequence, and these may overlap or not.

If it is taught that a peptide of the present invention may have a particular similarity relationship (e.g., markedly identical) to a reference protein (peptide), preferred peptides are those which comprise a sequence having that relationship to a core sequence of the reference protein (peptide), but with internal insertions or deletions in either sequence excluded. Even more preferred peptides are those whose entire sequence has that relationship, with the same exclusion, to a core sequence of that reference protein (peptide).

Library

The term “library” generally refers to a collection of chemical or biological entities which are related in origin, structure, and/or function, and which can be screened simultaneously for a property of interest.

Libraries may be classified by how they are constructed (natural vs. artificial diversity; combinatorial vs. noncombinatorial), how they are screened (hybridization, expression, display), or by the nature of the screened library members (peptides, nucleic acids, etc.).

In a “natural diversity” library, essentially all of the diversity arose without human intervention. This would be true, for example, of messenger RNA extracted from a non-engineered cell.

In a “synthetic diversity” library, essentially all of the diversity arose deliberately as a result of human intervention. This would be true for example of a combinatorial library; note that a small level of natural diversity could still arise as a result of spontaneous mutation. It would also be true of a noncombinatorial library of compounds collected from diverse sources, even if they were all natural products.

In a “non-natural diversity” library, at least some of the diversity arose deliberately through human intervention.

In a “controlled origin” library, the source of the diversity is limited in same way. A limitation might be to cells of a particular individual, to a particular species, or to a particular genus, or, more complexly, to individuals of a particular species who are of a particular age, sex, physical condition, geographical location, occupation and/or familial relationship. Alternatively or additionally, it might be to cells of a particular tissue or organ. Or it could be cells exposed to particular pharmacological, environmental, or pathogenic conditions. Or the library could be of chemicals, or a particular class of chemicals, produced by such cells.

In a “controlled structure” library, the library members are deliberately limited by the production conditions to particular chemical structures. For example, if they are oligomers, they may be limited in length and monomer composition, e.g. hexapeptides composed of the twenty genetically encoded amino acids.

Hybridization Library

In a hybridization library, the library members are nucleic acids, and are screened using a nucleic acid hybridization probe. Bound nucleic acids may then be amplified, cloned, and/or sequenced.

Expression Library

In an expression library, the screened library members are gene expression products, but one may also speak of an underlying library of genes encoding those products. The library is made by subcloning DNA encoding the library members (or portions thereof) into expression vectors (or into cloning vectors which subsequently are used to construct expression vectors), each vector comprising an expressible gene encoding a particular library member, introducing the expression vectors into suitable cells, and expressing the genes so the expression products are produced.

In one embodiment, the expression products are secreted, so the library can be screened using an affinity reagent, such as an antibody or receptor. The bound expression products may be sequenced directly, or their sequences inferred by, e.g., sequencing at least the variable portion of the encoding DNA.

In a second embodiment, the cells are lysed, thereby exposing the expression products, and the latter are screened with the affinity reagent.

In a third embodiment, the cells express the library members in such a manner that they are displayed on the surface of the cells, or on the surface of viral particles produced by the cells. (See display libraries, below).

In a fourth embodiment, the screening is not for the ability of the expression product to bind to an affinity reagent, but rather for its ability to alter the phenotype of the host cell in a particular detectable manner. Here, the screened library members are transformed cells, but there is a first underlying library of expression products which mediate the behavior of the cells, and a second underlying library of genes which encode those products.

Display Library

In a display library, the library members are each conjugated to, and displayed upon, a support of some kind. The support may be living (a cell or virus), or nonliving (e.g., a bead or plate).

If the support is a cell or virus, display will normally be effectuated by expressing a fusion protein which comprises the library member, a carrier moiety allowing integration of the fusion protein into the surface of the cell or virus, and optionally a lining moiety. In a variation on this theme, the cell coexpresses a first fusion comprising the library member and a linking moiety L1, and a second fusion comprising a linking moiety L2 and the carrier moiety. L1 and L2 interact to associate the first fusion with the second fusion and hence, indirectly, the library member with the surface of the cell or virus.

Soluble Library

In a soluble library, the library members are free in solution. A soluble library may be produced directly, or one may first make a display library and then release the library members from their supports.

Encapsulated Library

In an encapsulated library, the library members are inside cells or liposomes. Generally speaking, encapsulated libraries are used to store the library members for future use; the members are extracted in some way for screening purposes. However, if they differentially affect the phenotype of the cells, they may be screened indirectly by screening the cells.

cDNA Library

A cDNA library is usually prepared by extracting RNA from cells of particular origin, fractionating the RNA to isolate the messenger RNA (mRNA has a poly(A) tail, so this is usually done by oligo-dT affinity chromatography), synthesizing complementary DNA (cDNA) using reverse transcriptase, DNA polymerase, and other enzymes, subcloning the cDNA into vectors, and introducing the vectors into cells. Often, only mRNAs or cDNAs of particular sizes will be used, to make it more likely that the cDNA encodes a functional polypeptide.

A cDNA library explores the natural diversity of the transcribed DNAs of cells from a particular source. It is not a combinatorial library.

A cDNA library may be used to make a hybridization library, or it may be used as an (or to make) expression library.

Genomic DNA Library

A genomic DNA library is made by extracting DNA from a particular source, fragmenting the DNA, isolating fragments of a particular size range, subcloning the DNA fragments into vectors, and introducing the vectors into cells.

Like a cDNA library, a genomic DNA library is a natural diversity library, and not a combinatorial library. A genomic DNA library may be used the same way as a cDNA library.

Synthetic DNA Library

A synthetic DNA library may be screened directly (as a hybridization library), or used in the creation of an expression or display library of peptides/proteins.

Combinatorial Libraries

The term “combinatorial library” refers to a library in which the individual members are either systematic or random combinations of a limited set of basic elements, the properties of each member being dependent on the choice and location of the elements incorporated into it. Typically, the members of the library are at least capable of being screened simultaneously. Randomization may be complete or partial; some positions may be randomized and others predetermined, and at random positions, the choices may be limited in a predetermined manner. The members of a combinatorial library may be oligomers or polymers of some kind, in which the variation occurs through the choice of monomeric building block at one or more positions of the oligomer or polymer, and possibly in terms of the connecting linkage, or the length of the oligomer or polymer, too. Or the members may be nonoligomeric molecules with a standard core structure, like the 1,4-benzodiazepine structure, with the variation being introduced by the choice of substituents at particular-variable sites on the core structure. Or the members may be nonoligomeric molecules assembled like a jigsaw puzzle, but wherein each piece has both one or more variable moieties (contributing to library diversity) and one or more constant moieties (providing the functionalities for coupling the piece in question to other pieces).

Thus, in a typical combinatorial library, chemical building blocks are at least partially randomly combined into a large number (as high as 10¹⁵) of different compounds, which are then simultaneously screened for binding (or other) activity against one or more targets.

In a “simple combinatorial library”, all of the members belong to the same class of compounds (e.g., peptides) and can be synthesized simultaneously. A “composite combinatorial library” is a mixture of two or more simple libraries, e.g., DNAs and peptides, or peptides, peptoids, and PNAs, or benzodiazepines and carbamates. The number of component simple libraries in a composite library will, of course, normally be smaller than the average number of members in each simple library, as otherwise the advantage of a library over individual synthesis is small.

Libraries of thousands, even millions, of random oligopeptides have been prepared by chemical synthesis (Houghten et al., Nature, 354:84-6(1991)), or gene expression (Marks et al., J Mol Biol, 222:581-97(1991)), displayed on chromatographic supports (Lam et al., Nature, 354:82-4(1991)), inside bacterial cells (Colas et al., Nature, 380:548-550(1996)), on bacterial pili (Lu, Bio/Technology, 13:366-372(1990)), or phage (Smith, Science, 228:1315-7(1985)), and screened for binding to a variety of targets including antibodies (Valadon et al., J Mol Biol, 261:11-22(1996)), cellular proteins (Schmitz et al., J Mol Biol, 260:664-677(1996)), viral proteins (Hong and Boulanger, Embo J, 14:4714-4727(1995)), bacterial proteins (Jacobsson and Frykberg, Biotechniques, 18:878-885(1995)), nucleic acids (Cheng et al., Gene, 171:1-8(1996)), and plastic (Siani et al., J Chem Inf Comput Sci, 34:588-593(1994)).

Libraries of proteins (Ladner, U.S. Pat. No. 4,664,989), peptoids (Simon et al., Proc Natl Acad Sci U S A, 89:9367-71(1992)), nucleic acids (Ellington and Szostak, Nature, 246:818(1990)), carbohydrates, and small organic molecules (Eichler et al., Med Res Rev, 15:481-96(1995)) have also been prepared or suggested for drug screening purposes.

The first combinatorial libraries were composed of peptides or proteins, in which all or selected amino acid positions were randomized. Peptides and proteins can exhibit high and specific binding activity, and can act as catalysts. In consequence, they are of great importance in biological systems.

Nucleic acids have also been used in combinatorial libraries. Their great advantage is the ease with which a nucleic acid with appropriate binding activity can be amplified. As a result, combinatorial libraries composed of nucleic acids can be of low redundancy and hence, of high diversity.

There has also been much interest in combinatorial libraries based on small molecules, which are more suited to pharmaceutical use, especially those which, like benzodiazepines, belong to a chemical class which has already yielded useful pharmacological agents. The techniques of combinatorial chemistry have been recognized as the most efficient means for finding small molecules that act on these targets. At present, small molecule combinatorial chemistry involves the synthesis of either pooled or discrete molecules that present varying arrays of functionality on a common scaffold. These compounds are grouped in libraries that are then screened against the target of interest either for binding or for inhibition of biological activity.

The size of a library is the number of molecules in it. The simple diversity of a library is the number of unique structures in it. There is no formal minimum or maximum diversity. If the library has a very low diversity, the library has little advantage over just synthesizing and screening the members individually. If the library is of very high diversity, it may be inconvenient to handle, at least without automatizing the process. The simple diversity of a library is preferably at least 10, 10E2, 10E3, 10E4, 10E6, 10E7, 10E8 or 10E9, the higher the better under most circumstances. The simple diversity is -usually not more than 10E15, and more usually not more than 10E10.

The average sampling level is the size divided by the simple diversity. The expected average sampling level must be high enough to provide a reasonable assurance that, if a given structure were expected, as a consequence of the library design, to be present, that the actual average sampling level will be high enough so that the structure, if satisfying the screening criteria, will yield a positive result when the library is screened. Thus, the preferred average sampling level is a function of the detection limit, which in turn is a function of the strength of the signal to be screened.

There are more complex measures of diversity than simple diversity. These attempt to take into account the degree of structural difference between the various unique sequences. These more complex measures are usually used in the context of small organic compound libraries, see below.

The library members may be presented as solutes in solution, or immobilized on some form of support. In the latter case, the support may be living (cell, virus) or nonliving (bead, plate, etc.). The supports may be separable (cells, virus particles, beads) so that binding and nonbinding members can be separated, or nonseparable (plate). In the latter case, the members will normally be placed on addressable positions on the support. The advantage of a soluble library is that there is no carrier moiety that could interfere with the binding of the members to the support. The advantage of an immobilized library is that it is easier to identify the structure of the members which were positive.

When screening a soluble library, or one with a separable support, the target is usually immobilized. When screening a library on a nonseparable support, the target will usually be labeled.

Oligonucleotide Libraries

An oligonucleotide library is a combinatorial library, at least some of whose members are single-stranded oligonucleotides having three or more nucleotides connected by phosphodiester or analogous bonds. The oligonucleotides may be linear, cyclic or branched, and may include non-nucleic acid moieties. The nucleotides are not limited to the nucleotides normally found in DNA or RNA. For examples of nucleotides modified to increase nuclease resistance and chemical stability of aptamers, see Chart 1 in Osborne and Ellington, Chem. Rev., 97: 349-70 (1997). For screening of RNA, see Ellington and Szostak, Nature, 346: 818-22 (1990).

There is no formal minimum or maximum size for these oligonucleotides. However, the number of conformations which an oligonucleotide can assume increases exponentially with its length in bases. Hence, a longer oligonucleotide is more likely to be able to fold to adapt itself to a protein surface. On the other hand, while very long molecules can be synthesized and screened, unless they provide a much superior affinity to that of shorter molecules, they are not likely to be found in the selected population, for the reasons explained by Osborne and Ellington (1997). Hence, the libraries of the present invention are preferably composed of oligonucleotides having a length of 3 to 100 bases, more preferably 15 to 35 bases. The oligonucleotides in a given library may be of the same or of different lengths.

Oligonucleotide libraries have the advantage-that libraries of very high diversity (e.g., 10¹⁵) are feasible, and binding molecules are readily amplified in vitro by polymerase chain reaction (PCR). Moreover, nucleic acid molecules can have very high specificity and affinity to targets.

In a preferred embodiment, this invention prepares and screens oligonucleotide libraries by the SELEX method, as described in King and Famulok, Molec. Biol. Repts., 20: 97-107 (1994); L. Gold, C. Tuerk. Methods of producing nucleic acid ligands, U.S. Pat. No. 5,595,877; Oliphant et al. Gene 44:177 (1986).

The term “aptamer” is conferred on those oligonucleotides which bind the target protein. Such aptamers may be used to characterize the target protein, both directly (through identification of the aptamer and the points of contact between the aptamer and the protein) and indirectly (by use of the aptamer as a ligand to modify the chemical reactivity of the protein).

In a classic oligonuclotide, each nucleotide (monomeric unit) is composed of a phosphate group, a sugar moiety, and either a purine or a pyrimidine base. In DNA, the sugar is deoxyribose and in RNA it is ribose. The nucleotides are linked by 5′-3′ phosphodiester bonds.

The deoxyribose phosphate backbone of DNA can be modified to increase resistance to nuclease and to increase penetration of cell membranes. Derivatives such as mono- or dithiophosphates, methyl phosphonates, boxanophosphates, formacetals, carbamates, siloxanes, and dimethylenethio- sulfoxideo- and-sulfono- linked species are known in the art.

Peptide Library

A peptide is composed of a plurality of amino acid residues joined together by peptidyl (—NHCO—) bonds. A biogenic peptide is a peptide in which the residues are all genetically encoded amino acid residues; it is not necessary that the biogenic peptide actually be produced by gene express ion.

Amino acids are the basic building blocks with which peptides and proteins are constructed. Amino acids possess both an amino group (—NH₂) and a carboxylic acid group (—COOH). Many amino acids, but not all, have the alpha amino acid structure NH₂—CHR—COOH, where R is hydrogen, or any of a variety of functional groups.

Twenty amino acids are genetically encoded: Alanine, Arginine, Asparagine, Aspartic Acid, Cysteine, Glutamic Acid, Glutamine, Glycine, Histidine, Isoleucine, Leucine, Lysine, Methionine, Phenylalanine, Proline, Serine, Threonine, Tryptophan, Tyrosine, and Valine. Of these, all save Glycine are optically isomeric, however, only the L-form is found in humans. Nevertheless, the D-forms of these amino acids do have biological significance; D-Phe, for example, is a known analgesic.

Many other amino acids are also known, including: 2-Aminoadipic acid; 3-Aminoadipic acid; beta-Aminopropionic acid; 2-Aminobutyric-acid; 4-Aminobutyric acid (Piperidinic acid) ;6-Aminocaproic acid; 2-Aminoheptanoic acid; 2-Aminoisobutyric acid, 3-Aminoisobutyric acid; 2-Aminopimelic acid; 2,4-Diaminobutyric acid; Desmosine; 2,2′-Diaminopimelic acid; 2,3-Diaminopropionic acid; N-Ethylglycine; N-Ethylasparagine; Hydroxylysine; allo-Hydroxylysine; 3-Hydroxyproline; 4-Hydroxyproline; Isodesmosine; allo-Isoleucine; N-Metlylglycine (Sarcosine); N-Methylisoleucine; N-Methylvaline; Iorvaline; Norleucine; and Ornithine.

Peptides are constructed by condensation of amino acids and/or smaller peptides. The amino group of one amino acid (or peptide) reacts with the carboxylic acid group of a second amino acid (or peptide) to form a peptide (—NHCO—) bond, releasing one molecule of water. Therefore, when an amino acid is incorporated into a peptide, it should, technically speaking, be referred to as an amino acid residue. The core of that residue is the moiety which excludes the —NH and —CO linking functionalities which connect it to other residues. This moiety consists of one or more main chain atoms (see below) and the attached side chains.

The main chain moiety of each amino acid consists of the —NH and —CO linking functionalities and a core main chain moiety. Usually the latter is a single carbon atom. However, the core main chain moiety may include additional carbon atoms, and may also include nitrogen, oxygen or sulfur atoms, which together form a single chain. In a preferred embodiment, the core main chain atoms consist solely of carbon atoms.

The side chains are attached to the core main chain atoms. For alpha amino acids, in which the side chain is attached to the alpha carbon, the C-1, C-2 and N-2 of each residue form the repeating unit of the main chain, and the word “side chain” refers to the C-3 arid higher numbered carbon atoms and their substituents. It also includes H atoms attached to the main chain atoms.

Amino acids may be classified according to the number of carbon atoms which appear in the main chain between the carbonyl carbon and amino nitrogen atoms which participate in the peptide bonds. Among the 150 or so amino acids which occur in nature, alpha, beta, gamma and delta amino acids are known. These have 1-4 intermediary carbons. Only alpha amino acids occur in proteins. Proline is a special case of an alpha amino acid; its side chain also binds to the peptide bond nitrogen.

For beta and higher order amino acids, there is a choice as to which main chain core carbon a side chain other than H is attached to. The preferred attachment site is the C-2 (alpha) carbon, i.e., the one adjacent to the carboxyl carbon of the —CO linking functionality. It is also possible for more than one main chain atom to carry a side chain other than H. However, in a preferred- embodiment, only one main chain core atom carries a side chain other than H.

A main chain carbon atom may carry either one or two side chains; one is more common. A side chain may be attached to a main chain carbon atom by a single or a double bond; the former is more common.

A simple combinatorial peptide library is one whose members are peptides having three or more amino acids connected via peptide bonds.

The peptides may be linear, branched, or cyclic, and may covalently or noncovalently include nonpeptidyl moieties. The amino acids are not limited to the naturally occurring or to the genetically encoded amino acids.

A biased peptide library is one in which one or more (but not all) residues of the peptides are constant residues.

Cyclic Peptides

Many naturally occurring peptides are cyclic. Cyclization is a common mechanism for stabilization of peptide conformation thereby achieving improved association of the peptide with its ligand and hence improved biological activity. Cyclization is usually achieved by intra-chain cystine formation, by formation of peptide bond between side chains or between N— and C-terminals. Cyclization was usually achieved by peptides in solution, but several publications have appeared that describe cyclization of peptides on beads.

A peptide library may be an oligopeptide library or a protein library.

Oligopeptides

Preferably, the oligopeptides are at least five, six, seven or eight amino acids in length. Preferably, they are composed of less than 50, more preferably less than 20 amino acids.

In the case of an oligopeptide library, all or just some of the residues may be variable. The oligopeptide may be unconstrained, or constrained to a particular conformation by, e.g., the participation of constant cysteine residues in the formation of a constraining disulfide bond.

Proteins

Proteins, like oligopeptides, are composed of a plurality of amino acids, but the term protein is usually reserved for longer peptides, which are able to fold into a stable conformation. A protein may be composed of two or more polypeptide chains, held together by covalent or noncovalent crosslinks. These may occur in a homooligomeric or a heterooligomeric state.

A peptide is considered a protein if it (1) is at least 50 amino acids long, or (2) has at least two stabilizing covalent crosslinks (e.g., disulfide bonds). Thus, conotoxins are considered proteins.

Usually, the proteins of a protein library will be characterizable as having both constant residues (the same for all proteins in the library) and variable residues (which vary from member to member). This is simply because, for a given range of variation at each position, the sequence space (simple diversity) grows exponentially with the number of residue positions, so at some point it becomes inconvenient for all residues of a peptide to be variable positions. Since proteins are usually larger than oligopeptides, it is more common for protein libraries than oligopeptide libraries to feature variable positions.

In the case of a protein library, it is desirable to focus the mutations at those sites which are tolerant of mutation. These may be determined by alanine scanning mutagenesis or by comparison of the protein sequence to that of homologous proteins of similar activity. It is also more likely that mutation of surface residues will directly affect binding. Surface residues may be determined by inspecting a 3D structure of the protein, or by labeling the surface and then ascertaining which residues have received labels. They may also be inferred by identifying regions of high hydrophilicity within the protein.

Because proteins are often altered at some sites but not others, protein libraries can be considered a special case of the biased peptide library.

There are several reasons that one might screen a protein library instead of an oligopeptide library, including (1) a particular protein, mutated in the library, has the desired activity to some degree already, and (2) the oligopeptides are not expected to have a sufficiently high affinity or specificity since they do not have a stable conformation.

When the protein library is based on a parental protein which does not have the desired activity, the parental protein will usually be one which is of high stability (melting point >=50 deg. C.) and/or possessed of hypervariable regions.

The variable domains of an antibody possess hypervariable regions and hence, in some embodiments, the protein library comprises members which comprise a mutant of VH or VL chain, or a mutant of an antigen-specific binding fragment of such a chain. VH and VL chains are usually each about 110 amino acid residues, and are held in proximity by a disulfide bond between the adjoing CL and CH1 regions to form a variable domain. Together, the VH, VL, CL and CH1 form an Fab fragment.

In human heavy chains, the hypervariable regions are at 31-35, 49-65, 98-111 and 84-88, but only the first three are involved in antigen binding. There is variation among VH and VL chains at residues outside the hypervariable regions, but to a much lesser degree.

A sequence is considered a mutant of a VH or VL chain if it is at least 80% identical to a naturally occurring VH or VL chain at all residues outside-the hypervariable region.

In a preferred embodiment, such antibody library members comprise both at least one VH chain and at least one VL chain, at least one of which is a mutant chain, and which chains may be derived from the same or different antibodies. The VH and VL chains may be covalently joined by a suitable linker moiety, as in a “single chain antibody”, or they may be noncovalently joined, as in a naturally occurring variable domain.

If the joining is noncovalent, and the library is displayed on cells or virus, then either the VH or the VL chain may be fused to the carrier surface/coat protein. The complementary chain may be co-expressed, or added exogenously to the library.

The members may further comprise some or all of an antibody constant heavy and/or constant light chain, or a mutant thereof.

Peptoid Library

A peptoid is an analogue of a peptide in which one or more of the peptide bonds (—NH—CO—) are replaced by pseudopeptide bonds, which may be the same or different. It is not necessary that all of the peptide bonds be replaced, i.e., a peptoid may include one or more conventional amino acid residues, e.g., proline

A peptide bond has two small divalent linker elements, —NH— and —CO—. Thus, a preferred class of pseudopeptide bonds are those which consist of two small divalent linker elements. Each may be chosen independently from the group consisting of amine (—NH—), substituted amine (—NR—), carbonyl (—CO—), thiocarbonyl (—CS—),methylene (—CH2—), monosubstituted methylene (—CHR—), disubstituted methylene (—CR1R2—), ether (—O—) and thioether (—S—). The more preferred pseudopeptide bonds include:
N-modified —NRCO—
Carba Ψ —CH₂—CH₂—
Depsi Ψ —CO—O—
Hydroxyethylene Ψ —CHOH—CH₂—
Ketomethylene Ψ —CO—CH₂—
Methylene-Oxy —CH₂—O—
Reduced —CH₂—NH—
Thiomethylene —CH₂—S—
Thiopeptide —CS—NH—
Retro-Inverso —CO—NH—

A single peptoid molecule may include more than one kind of pseudopeptide bond.

For the purposes of introducing diversity into a peptoid library, one may vary (1) the side chains attached to the core main chain atoms of the monomers linked by the pseudopeptide bonds, and/or (2) the side chains (e.g., the —R of an —NRCO—) of the pseudopeptide bonds. Thus, in one embodiment, the monomeric units which are not amino acid residues are of the structure —NR1—CR2—CO—, where at least one of R1 and R2 are not hydrogen. If there is variability in the pseudopeptide bond, this is most conveniently done by using an —NRCO— or other pseudopeptide bond with an R group, and varying the R group. In this event, the R group will usually be any of the side chains characterizing the amino acids of peptides, as previously discussed.

If the R group of the pseudopeptide bond is not variable, it will usually be small, e.g., not more than 10 atoms (e.g., hydroxyl, amino, carboxyl, methyl, ethyl, propyl).

If the conjugation chemistries are compatible, a simple combinatorial library may include both peptides and peptoids.

Peptide Nucleic Acid Library

A PNA oligomer is here defined as one comprising a plurality of units, at least one of which is a PNA monomer which comprises a side chain comprising a nucleobase. For nucleobases, see U.S. Pat. No. 6,077,835.

The classic PNA oligomer is composed of (2-aminoethyl)glycine units, with nucleobases attached by methylene carbonyl linkers. That is, it has the structure
H—(—HN—CH₂—CH₂—N(—CO—CH₂ —B)—CH₂—CO—), —OH
where the outer parenthesized substructure is the PNA monomer.

In this structure, the nucleobase B is separated from the backbone N by three bonds, and the points of attachment of the side chains are separated by six bonds. The nucleobase may be any of the bases included in the nucleotides discussed in connection with oligonucleotide libraries. The bases of nucleotides A, G, T, C and U are preferred.

A PNA oligomer may further comprise one or more amino acid residues, especially glycine and proline.

One can readily envision related molecules in which (1) the —COCH2— linker is replaced by another linker, especially one composed of two small divalent linkers as defined previously, (2) a side chain is attached to one of the three main chain carbons not participating in the peptide bond (either instead or in addition to the side chain attached to the N of the classic PNA); and/or (3) the peptide bonds are replaced by pseudopeptide bonds as disclosed previously in the context of peptoids.

PNA oligomer libraries have been made; see e.g. Cook, U.S. Pat. No. 6,204,326.

Small Organic Compound Library

The small organic compound library (“compound library”, for short) is a combinatorial library whose members are suitable for use as drugs if, indeed, they have the ability to-mediate a biological activity of the target protein.

Peptides have certain disadvantages as drugs. These include susceptibility to degradation by serum proteases, and difficulty in penetrating cell membranes. Preferably, all or most of the compounds of the compound library avoid, or at least do not'suffer to the same degree, one or more of the pharmaceutical disadvantages of peptides.

In designing a compound library, it is helpful to bear in mind the methods of molecular modification typically used to obtain new drugs. Three basic kinds of modification may be identified: disjunction, in which a lead drug is simplified to identify its component pharmacophoric moieties; conjunction, in which two or more known pharmacophoric moieties, which may be the same or different, are associated, covalently or noncovalently, to form a new drug; and alteration, in which one moiety is replaced by another which may be similar or different, but which is not in effect a disjunction or conjunction. The use of the terms “disjunction”, “conjunction” and “alteration” is intended only to connote the structural relationship of the end product to the original leads, and not how the new drugs are actually synthesized, although it is possible that the two are the same The process of disjunction is illustrated by the evolution of neostigmine (1931) and edrophonium (1952) from physostigmine (1925). Subsequent conjunction is illustrated by demecarium (1956) and ambenonium (1956).

Alterations may modify the size, polarity, or electron distribution of an original moiety. Alterations include ring closing or opening, formation of lower or higher homologues, introduction or saturation of double bonds, introduction of optically active centers, introduction, removal or replacement of bulky groups, isosteric or bioisosteric substitution, changes in the position or orientation of a group, introduction of alkylating groups, and introduction, removal or replacement of groups with a view toward inhibiting or promoting inductive (electrostatic) or conjugative (resonance) effects.

Thus, the substituents may include electron acceptors and/or electron donors. Typical electron donors (+I) include —CH₃, —CH₂R, —CHR₂, —CR₃ and —COO—. Typical electron acceptors (−I) include —NH3+, —NR3+, —NO₂, —CN, —COOH, —COOR, —CHO, —COR, —COR, —F, —Cl, —Br, —OH, —OR, —SH, —SR, —CH═CH₂, —CR═CR₂, and —C═CH.

The substituents may also include those which increase or decrease electronic density in conjugated systems. The former (+R) groups include —CH₃, —CR₃, —F, —Cl, —Br, —I, —OH, —OR, —OCOR, —SH, —SR, —NH₂, —NR₂, and —NHCOR. The later (−R) groups include —NO₂, —CN, —CHC, —COR, —COOH, —COOR, —CONH₂, —SO₂R and —CF₃.

Synthetically speaking, the modifications may be achieved by a variety of unit processes, including nucleophilic and electrophilic substitution, reduction and oxidation, addition elimination, double bond cleavage, and cyclization.

For the purpose of constructing a library, a compound, or a family of compounds, having one or more pharmacological activities (which need not be related to the known or suspected activities of the target protein), may be disjoined into two or more known or potential pharmacophoric moieties. Analogues of each of these moieties may be identified, and mixtures of these analogues reacted so as to reassemble compounds which have some similarity to the original lead compound. It is not necessary that all members of the library possess moieties analogous to all of the moieties of the lead compound.

The design of a library may be illustrated by the example of the benzodiazepines. Several benzodiazepine drugs, including chlordiazepoxide, diazepam and oxazepam, have been used as anti-anxiety drugs. Derivatives of benzodiazepines have widespread biological activities; derivatives have been reported to act not only as anxiolytics, but also as anticonvulsants; cholecystokinin (CCK) receptor subtype A or B, kappa opioid receptor, platelet activating factor, and HIV transactivator Tat antagonists, and GPIIbIIa, reverse transcriptase and ras farnesyltransferase inhibitors.

The benzodiazepine structure has been disjoined into a 2-aminobenzophenone, an amino acid, and an alkylating agent. See Bunin, et al., Proc. Nat. Acad. Sci. USA, 91:4708 (1994). Since only a few 2-aminobenzophenone derivatives are commercially available, it was later disjoined into 2-aminoarylstannane, an acid chloride, an amino acid, and an alkylating agent. Bunin, et al., Meth. Enzymol., 267:4.48 (1996). The arylstannane may be considered the core structure upon which the other moieties are substituted, or all four may be considered equals which are conjoined to make each library member:

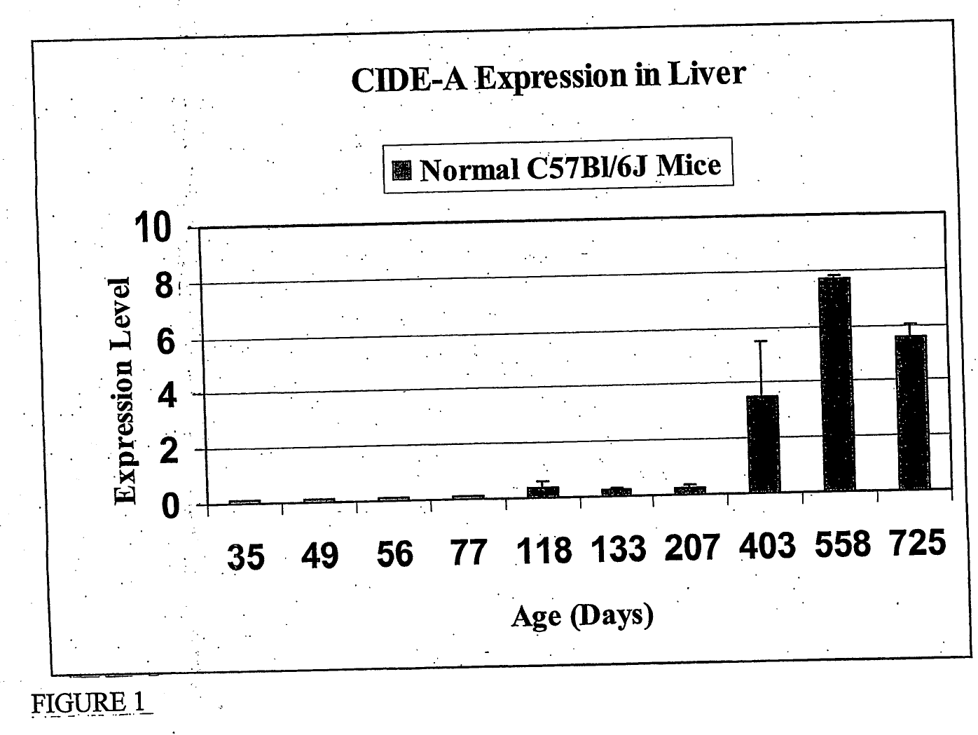

A basic library synthesis plan and member structure is shown in FIG. 1 of Fowlkes, et al., U.S. Ser. No. 08/740,671, incorporated by reference in its entirety. The acid chloride building block introduces variability at the R¹ site. The R² site is introduced by the amino acid, and the R³ site by the alkylating agent. The R⁴ site is inherent in the arylstannane. Bunin, et al. generated a 1,4-benzodiazepine library of 11,200 different derivatives prepared from 20 acid chlorides, 35 amino acids, and 16 alkylating agents. (No diversity was introduced at R⁴; this group was used to couple the molecule to a solid phase.) According to the Available Chemicals Directory (HDL Information Systems, San Leandro Calif.), over 300 acid chlorides, 80 Fmoc-protected amino acids and 800 alkylating agents were available for purchase (and more, of course, could be synthesized). The particular moieties used were chosen to maximize structural dispersion, while limiting the numbers to those conveniently synthesized in the wells of a microtiter plate. In choosing between structurally similar compounds, preference was given to the least substituted compound.

The variable elements included both aliphatic and aromatic groups. Among the aliphatic groups, both acyclic and cyclic (mono- or poly-) structures, substituted or not, were tested. (While all of the acyclic groups were linear, it would have been feasible to introduce a branched aliphatic). The aromatic groups featured either single and multiple rings, fused or not, substituted or not, and with heteroatoms or not. The secondary substituents included —NH₂, —OH, —OMe, —CN, —Cl, —F, and —COOH. While not used, spacer moieties, such as —O—, —S—, —OO—, —CS—, —NH—, and —NR—, could have been incorporated.

Bunin et al. suggest that instead of using a 1,4-benzodiazepine as a core structure, one may instead use a 1,4-benzodiazepine-2,5-dione structure.

As noted by Bunin et al., it is advantageous, although not necessary, to use a linkage strategy which leaves no trace of the linking functionality, as this permits construction of a more diverse library.

Other combinatorial nonoligomeric compound libraries known or suggested in the art have been based on carbamates, mercaptoacylated pyrrolidines, phenolic agents, aminimides, N-acylamino ethers (made from amino alcohols, aromatic hydroxy acids, and carboxylic acids), N-alkylamino ethers (made from aromatic hydroxy acids, amino alcohols and aldehydes) 1,4-piperazines, and 1,4-piperazine-6-ones.

DeWitt, et al., Proc. Nat. Acad. Sci. (USA), 90 :6909-13 (1993) describe the simultaneous but separate, synthesis of 40 discrete hydantoins and 40 discrete benzodiazepiaes. They carry out their synthesis on a solid support (inside a gas dispersion tube), in an array format, as opposed to other conventional simultaneous synthesis techniques (e.g., in a well, or on a pin). The hydantoins were synthesized by first simultaneously deprotecting and then treating each of five amino acid resins with each of eight isocyanates. The benzodiazepines were synthesized by treating each of five deprotected amino acid resins with each of eight 2-amino benzophenone imines.

Chen, et al., J. Am. Chem. Soc., 116:2661-62 (1994) described the preparation of a pilot (9 member) combinatorial library of formate esters. A polymer bead-bound aldehyde preparation was “split” into three aliquots, each reacted with one of three different ylide reagents. The reaction products were combined, and then divided into three new aliquots, each of which was reacted with a different Michael donor. Compound identity was found to be determinable on a single bead basis by gas chromatography/mass spectroscopy analysis.

Holmes, U.S. Pat. No. 5,549,974 (1996) sets forth methodologies for the combinatorial synthesis of libraries of thiazolidinones and metathiazanones. These libraries are made by combination of amines, carbonyl compounds, and thiols under cyclization conditions.

Ellman, U.S. Pat. No. 5, 545,568 (1996) describes combinatorial synthesis of benzodiazepines, prostaglandins, beta-turn mimetics, and glycerol-based compounds. See also Ell man, U.S. Pat. No. 5,288,514.

Summerton, U.S. Pat. No. 5,506,337 (1996) discloses methods of preparing a combinatorial library formed predominantly of morpholino subunit structures.

Heterocylic combinatorial libraries are reviewed generally in Nefzi, et al., Chem. Rev., 97:449-472 (1997)

For pharmacological classes, see, e.g., Goth, Medical Pharmacology: Principles and Concepts (C.V. Mosby Co.: 8th ed. 1976); Korolkovas and Burckhalter, Essentials of Medicinal Chemistry (John Wiley & Sons, Inc.: 1976). For synthetic methods, see, e.g., Warren, Organic Synthesis: The Disconnection Approach (John Wiley & Sons, Ltd.: 1982); Fuson, Reactions of Organic Compounds (John Wiley & Sons: 1966); Payne and Payne, How to do an Organic Synthesis (Allyn and Bacon, Inc.: 1969); Greene, Protective Groups in Organic Synthesis (Wiley-Interscience). For selection of substituents, see e.g., Hansch and Leo, Substituent Constants for Correlation Analysis in Chemistry and Biology (John Wiley & Sons: 1979).

The library is preferably synthesized so that the individual members remain identifiable so that, if a member is shown to be active, it is not necessary to analyze it. Several methods of identification have been proposed, including:
(1) encoding, i.e., the attachment to each member of an identifier moiety which is more really identified than the member proper. This has the disadvantage that the tag may itself influence the activity of the conjugate.
(2) spatial addressing, e.g., each member is synthesized only at a particular coordinate on or in a matrix, or in a particular chamber. This might be, for example, the location of a particular pin, or a particular well on a microtiter plate, or inside a “tea bag”.
   The present invention is not limited to any particular form of identification.

However, it is possible to simply characterize those members of the library which are found to be active, based on the characteristic spectroscopic indicia of the various building blocks.

Solid phase synthesis permits greater control over which derivatives are formed. However, the solid phase could interfere with activity. To overcome this problem, some or all of the molecules of each member could be liberated, after synthesis but before screening.

Examples of candidate simple libraries which might be evaluated include derivatives of the following:

Cyclic Compounds Containing One Hetero Atom
Heteronitrogen
   pyrroles
      pentasubstituted pyrroles
   pyrrolidines
   pyrrolines
   prolines
   indoles
   beta-carbolines
   pyridines
      dihydropyridines
      1,4-dihydropyridines
      pyrido[2,3-d]pyrimidines
      tetrahydro-3H-imidazo[4,5-c]pyridines
   Isoquinolines
      tetrahydroisoquinolines
   quinolones
   beta-lactams
      azabicyclo[4.3.0]nonen-8-one amino acid
Heterooxygen
   furans
      tetrahydrofurans
         2,5-disubstituted tetrahydrofurans pyrans
      hydroxypyranones
      tetrahydroxypyranones
   gamma-butyrolactones
Heterosulfur
   sulfolenes

Cyclic Compounds with Two or More Hetero atoms
Multiple heteronitrogens
   imidazoles
   pyrazoles
   piperazines
      diketopiperazines
      arylpiperazines
      benzylpiperazines
   benzodiazepines
   1,4-benzodiazepine-2,5-diones
   hydantoins
      5-alkoxyhydantoins
   dihydropyrimidines
   1,3 -disubstituted-5,6-dihydopyrimidine-2,4-diones
   cyclic ureas
   cyclic thioureas
   quinazolines
      3 chiral 3-substituted-quinazoaine-2,4-diones
   triazoles
      1,2,3-triazoles
   purines
Heteronitrogen and Heterooxygen
   dikelomorpholines
   isoxazoles
   isoxazolines
Heteronitrogen and Heterosulfur
   thiazolidines
      N-axylthiazolidines
   dihydrothiazoles
      2-methylene-2,3-dihydrothiazates
      2-aminothiazoles
   thiophenes
      3-amino thiophenes
   4-thiazolidinones
   4-melathiazanones
   benzisothiazolones

For details on synthesis of libraries, see Nefzi, et al., Chem. Rev., 97:449-72 (1997), and references cited therein.

Pharmaceutical Methods and Preparations

The preferred animal subject of the present invention is a mammal By the term “mammal” is meant an individual belonging to the class Mammalia. The invention is particularly useful in the treatment of human subjects, although it is intended for veterinary and nutritional uses as well. Preferred nonhuman subjects are of the orders Primata (e.g., apes and monkeys), Artiodactyla or Perissodactyla (e.g., cows, pigs, sheep, horses, goats), Carnivora (e.g., cats, dogs), Rodenta (e.g., rats, mice, guinea pigs, hamsters), Lagomorpha (e.g., rabbits) or other pet, farm or laboratory mammals.

The term “protection”, as used herein, is intended to include “prevention,” “suppression” and “treatment.” “Prevention”, strictly speaking, involves administration of the pharmaceutical prior to the induction of the disease (or other adverse clinical condition). “Suppression” involves administration of the composition prior to the clinical appearance of the disease. “Treatment” involves administration of the protective composition after the appearance of the disease.

It will be understood that in human and veterinary medicine, it is not always possible to distinguish between “preventing” and “suppressing” since the ultimate inductive event or events may be unknown, latent, or the patient is not ascertained until well after the occurrence of the event or events. Therefore, unless qualified, the term “prevention” will be understood to refer to both prevention in the strict sense, and to suppression.

The preventative or prophylactic use of a pharmaceutical usually involves identifying subjects who are at higher risk than the general population of contracting the disease, and administering the pharmaceutical to them in advance of the clinical appearance of the disease. The effectiveness of such use is measured by comparing the subsequent incidence or severity of the disease, or of particular symptoms of the disease, in the treated subjects against that in untreated subjects of the same high risk group.

While high risk factors vary-from disease to disease, in general, these include (1) prior occurrence of the disease in one or more members of the same family, or, in the case of a contagious disease, in individuals with whom the subject has come into potentially contagious contact at a time when the earlier victim was likely to be contagious, (2) a prior occurrence of the disease in the subject, (3) prior occurrence of a related disease, or a condition known to increase the likelihood of the disease, in the subject; (4) appearance of a suspicious level of a marker of the disease, or a related disease or condition; (5) a subject who is immunologically compromised, e.g., by radiation treatment, HIV infection, drug use, etc., or (6) membership in a particular group (e.g., a particular age, sex, race, ethnic group, etc.) which has been epidemiologically associated with that disease.

In some cases, it may be desirable to provide prophylaxis for the general population, and not just a high risk group. This is most likely to be the case when essentially all are at risk of contracting the disease, the effects of the disease are serious, the therapeutic index of the prophylactic agent is high, and the cost of the agent is low.

A prophylaxis or treatment may be curative, that is, directed at the underlying cause of a disease, or ameliorative, that is, directed at the symptoms of the disease, especially those which reduce the quality of life.

It should also be understood that to be useful, the protection provided need not be absolute, provided that it is sufficient to carry clinical value. An agent which provides protection to a lesser degree than do competitive agents may still be of value if the other agents are ineffective for a particular individual, if it can be used in combination with other agents to enhance the level of protection, or if it is safer than competitive agents. It is desirable that there be a statistically significant (p=0.05 or less) improvement in the treated subject relative'to an appropriate untreated control, and -it is desirable that this improvement be at least 10%, more preferably at least 25%, still more preferably at least 50%, even more preferably at least 100%, in some indicia of the incidence or severity of the disease or of at least one symptom of the disease.

At least one of the drugs of the present invention may be administered, by any means that achieve theirs intended purpose, to protect a subject against a disease or other adverse condition. The form of administration may be systemic or topical. For example, administration of such a composition may be by various parenteral routes such as subcutaneous, intravenous, intradermal, intramuscular, intraperitoneal, intranasal, transdermal, or buccal routes. Alternatively, or concurrently, administration may be by the oral route. Parenteral administration can be by bolus injection or by gradual perfusion over, time.

A typical regimen comprises administration of an effective amount of the drug, administered over a period ranging from a single dose, to dosing over a period of hours, days, weeks, months, or years.

It is understood that the suitable dosage of a drug of the present invention will be dependent upon the age, sex, health, and weight of the recipient, kind of concurrent treatment, if any, frequency of treatment, and the nature of the effect desired. However, the most preferred dosage can be tailored to the individual subject, as is understood and determinable by one of skill in the art, without undue experimentation. This will typically involve adjustment of a standard dose, e.g., reduction of the dose if the patient has a low body weight.

Prior to use in humans, a drug will first be evaluated for safety and efficacy in laboratory animals. In human clinical studies, one would begin with a dose expected to be safe in humans, based on the preclinical data for the drug in question, and on customary doses for analogous drugs (if any). If this dose is effective, the dosage may be decreased, to determine the minimum effective dose, if desired. If this dose is ineffective, it will be cautiously increased, with the patients monitored for signs of side effects. See, e.g., Berkow et al, eds., The Merck Manual, 15th edition, Merck and Co., Rahway, N.J., 1987; Goodman et al., eds., Goodman and Gilman's The Pharmacological Basis of Therapeutics, 8th edition, Pergamon Press, Inc., Elmsford, N.Y., (1990); Avery's Drug Treatment: Principles and Practice of Clinical Pharmacology and Therapeutics, 3rd edition, ADIS Press, LTD., Williams and Wilkins, Baltimore, Md. (1987), Ebadi, Pharmacology, Little, Brown and Co., Boston, (1985), which references and references cited therein, are entirely incorporated herein by reference.

The total dose required for each treatment may be administered by multiple doses or in a single dose. The protein may be administered alone or in conjunction with other therapeutics directed to the disease or directed to other symptoms thereof.

Typical pharmaceutical doses, for adult humans, are in the range of 1 ng to 10 g per day, more often 1 mg to 1 g per day.

The appropriate dosage form will depend on the disease, the pharmaceutical, and the mode of administration; possibilities include tablets, capsules, lozenges, dental pastes, suppositories, inhalants, solutions, ointments and parenteral depots. See, e.g., Berker, supra, Goodman, supra, Avery, supra and Ebadi, supra, which are entirely incorporated herein by reference, including all references cited therein.

In the case of peptide drugs, the drug may be administered in the form of an expression vector comprising a nucleic acid encoding the peptide; such a vector, after incorporation into the genetic complement of a cell of the patient, directs synthesis of the peptide. Suitable vectors include genetically engineered poxviruses (vaccinia), adenoviruses, adeno-associated viruses, herpesviruses and lentiviruses which are or have been rendered nonpathogenic.

In addition to at least one drug as described herein, a pharmaceutical composition may contain suitable pharmaceutically acceptable carriers, such as excipients, carriers and/or auxiliaries which facilitate processing of the active compounds into preparations which can be used pharmaceutically. See, e.g., Berker, supra, Goodman, supra, Avery, supra and Ebadi, supra, which are entirely incorporated herein by reference, included all references cited therein.

Assay Compositions and Methods

Target Organism

The invention contemplates that it may be appropriate to ascertain or to mediate the biological activity of a substance of this invention in a target organism.

The target organism may be a plant, animal, or microorganism.

In the case of a plant, it may be an economic plant, in which case the drug may be intended to increase the disease, weather or pest resistance, alter the growth characteristics, or otherwise improve the useful characteristics or mute undesirable characteristics of the plant. Or it may be a weed, in which case the drug may be intended to kill or otherwise inhibit the growth of the plant, or to alter its characteristics to convert it from a weed to an economic plant. The plant may be a tree, shrub, crop, grass, etc. The plant may be an algae (which are in some cases also microorganisms), or a vascular plant, especially gymnosperms (particularly conifers) and angiosperms. Angiosperms may be monocots or dicots. The plants of greatest interest are rice, wheat, corn, alfalfa, soybeans, potatoes, peanuts, tomatoes, melons, apples, pears, plums, pineapples, fir, spruce, pine, cedar, and oak.

If the target organism is a microorganism, it may be algae, bacteria, fungi, or a virus (although the biological activity of a virus must be determined in a virus-infected cell). The microorganism may be human or other animal or plant pathogen, or it may be nonpathogenic. It may be a soil or water organism, or one which normally lives inside other living things.

If the target organism is an animal, it may be a vertebrate or a nonvertebrate animal. Nonvertebrate animals are chiefly of interest when they act as pathogens or parasites, and the drugs are intended to act as biocidic or biostatic agents. Nonvertebrate animals of interest include worms, mollusks, and arthropods.

The target organism may also be a vertebrate animal, i.e., a mammal, bird, reptile, fish or amphibian. Among mammals, the target animal preferably belongs to the order Primata (humans, apes and monkeys), Artiodactyla (e.g., cows, pigs, sheep, goats, horses), Rodenta (e.g., mice, rats) Lagomorpha (e.g., rabbits, hares), or Carnivora (e.g., cats, dogs). Among birds, the target animals are preferably of the orders Anseriformes (e.g., ducks, geese, swans) or Galliformes (e.g., quails, grouse, pheasants, turkeys and chickens). Among fish, the target animal is preferably of the order Clupeiformes (e.g., sardiries, shad, anchovies, whitefish, salmon).

Target Tissues

The term “target tissue” refers to any whole animal, physiological system, whole organ, part of organ, miscellaneous tissue, cell, or cell component (e.g., the cell membrane) of a target animal in which biological activity may be measured.

Routinely in mammals one would choose to compare and contrast the biological impact on virtually any and all tissues which express the subject receptor protein. The main tissues to use are: brain, heart, lung, kidney, liver, pancreas, skin, intestines, adipose, stomach, skeletal muscle, adrenal glands, breast, prostate, vasculature, retina, cornea, thyroid gland, parathyroid glands, thymus, bone marrow, bone, etc.

Another classification would be by cell type: B cells, T cells, macrophages, neutrophils, eosinophils, mast cells, platelets, megakaryocytes, erythrocytes, bone marrow stomal cells, fibroblasts, neurons, astrocytes, neuroglia, microglia, epithelial cells (from any organ, e.g. skin, breast, prostate, lung, intestines etc), cardiac muscle cells, smooth muscle cells, striated muscle cells, osteoblasts, osteocytes, chondroblasts, chondrocytes, keratinocytes, melanocytes, etc.

Of course, in the case of a unicellular organism, there is no distinction between the “target organism” and the “target tissue”.

Screening Assays

Assays intended to determine the binding or the biological activity of a substance are called preliminary screening assays.

Screening assays will typically be either in vitro (cell-free) assays (for binding to an immobilized receptor) or cell-based assays (for alterations in the phenotype of the cell). They will not involve screening of whole multicellular organisms, or isolated organs. The comments on diagnostic biological assays apply mutatis mutandis to screening cell-based assays.

In Vitro vs. In Vivo Assays

The term in vivo is descriptive of an event, such as binding or enzymatic action, which occurs within a living organism. The organism in question may, however, be genetically modified. The term in vitro refers to an event which occurs outside a living organism. Parts of an organism (e.g., a membrane, or an isolated biochemical) are used, together with artificial substrates and/or conditions. For the purpose of the present invention, the term in vitro excludes events occurring inside or on an intact cell, whether of a unicellular or multicellular organism.

In vivo assays include both cell-based assays, and organismic assays. The cell-based assays include both assays on unicellular organisms, and assays on isolated cells or cell cultures derived from multicellular organisms. The cell cultures may be mixed, provided that they are not organized into tissues or organs. The term organismic assay refers to assays on whole multicellular organisms, and assays on isolated organs or tissues of such organisms.

In vitro Diagnostic Methods and Reagents

The in vitro assays of the present invention may be applied to any suitable analyte-containing sample, and may be qualitative or quantitative in nature.

Sample

The sample will normally be a biological fluid, such as blood, urine, lymph, semen, milk, or cerebrospinal fluid, or a fraction or derivative thereof, or a biological tissue, in the form of, e.g., a tissue section or homogenate. However, the sample conceivably could be (or derived from) a food or beverage, a pharmaceutical ox diagnostic composition, soil, or surface or ground water. If a biological fluid or tissue, it may be taken from a human or other mammal, vertebrate or animal, or from a plant. The preferred sample is blood, or a fraction or derivative thereof.

Binding and Reaction Assays

The assay may be a binding assay, in which one step involves the binding of a diagnostic reagent to the analyte, or a reaction assay, which involves the reaction of a reagent with the analyte. The reagents used in a binding assay may be classified as to the nature of their interaction with analyte: (1) analyte analogues, or (2) analyte binding molecules (ABM). They may be labeled or insolubilized.

In a reaction assay, the assay may look for a direct reaction between the analyte and a reagent which is reactive with the analyte, or if the analyte is an enzyme or enzyme inhibitor, for a reaction catalyzed or inhibited by the analyte. The reagent may be a reactant, a catalyst, or an inhibitor for the reaction.

An assay may involve a cascade of steps in which the product of one step acts as the target for the next step. These steps may be binding steps, reaction steps, or a combination thereof.

Signal Producing System (SPS)

In order to detect the presence, or measure the amount, of an analyte, the assay must provide for a signal producing system (SPS) in which there is a detectable difference in the signal produced, depending on whether the analyte is present or absent (or, in a quantitative assay, on the amount of the analyte). The detectable signal may be one which is visually detectable, or one detectable only with instruments. Possible signals include production of colored or luminescent products, alteration of the characteristics (including amplitude or polarization) of absorption or emission of radiation by an assay component or product, and precipitation or agglutination of a component or product. The term “signal” is intended to include the discontinuance of an existing signal, or a change in the rate of change of an observable parameter, rather than a change in its absolute value. The signal may be monitored manually or automatically.

In a reaction assay, the signal is often a product of the reaction. In a binding assay, it is normally provided by a label borne by a labeled reagent.

Labels

The component of the signal producing system which is most intimately associated with the diagnostic reagent is called the “label”. A label may be, e.g., a radioisotope, a fluorophore, an enzyme, a co-enzyme, an enzyme substrate, an electron-dense compound, an agglutinable particle.

The radioactive isotope can be detected by such means as the use of a gamma counter or a scintillation counter or by autoradiography. Isotopes which are particularly useful for the purpose of the present invention include ³H, ¹²⁵I, ¹³¹I, ³⁵S, ¹⁴C, ³²P and ³³P. ¹²⁵I is preferred for antibody labeling.

The label may also be a fluorophore. When the fluorescently labeled reagent is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

Alternatively, fluorescence-emitting metals such as ¹²⁵Eu, or others of the lanthanide series, may be incorporated into a diagnostic reagent using such metal chelating groups as diethylenetriaminepentaacetic acid (DTPA) of ethylenediamine-tetraacetic acid (EDTA).

The label may also be a chemiluminescent compound. The presence of the chemiluminescently labeled reagent is then determined by detecting the presence of luminescence that arises during the course of a chemical reaction. Examples of particularly useful chemiluminescent labeling compounds are luminol, isolumino, theromatic acridinium ester, imidazole, acridinium salt and oxalate ester.

Likewise, a bioluminescent compound may be used for labeling. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Enzyme labels, such as horseradish peroxidase and alkaline phosphatase, are preferred. When an enzyme label is used, the signal producing system must also include a substrate for the enzyme If the enzymatic reaction product is not itself detectable, the SPS will include one or more additional reactants so that a detectable product appears.

An enzyme analyte may act as its own label if an enzyme inhibitor is used as a diagnostic reagent.

Binding Assay Formats

Binding assays may be divided into two basic types, heterogeneous and homogeneous. In heterogeneous assays, the interaction between the affinity molecule and the analyte does not affect the label, hence, to determine the amount or presence of analyte, bound label must be separated from free label. In homogeneous assays, the interaction does affect the activity of the label, and therefore analyte levels can be deduced without the need for a separation step.

In one embodiment, the ABM is insolubilized by coupling it to a macromolecular support, and analyte in the sample is allowed to compete with a known quantity of a labeled or specifically labelable analyte analogue. The “analyte analogue” is a molecule capable of competing with analyte for binding to the ABM, and the term is intended to include analyte itself. It may be labeled already, or it may be labeled subsequently by specifically binding the label to a moiety differentiating the analyte analogue from analyte. The solid and liquid phases are separated, and the labeled analyte analogue in one phase is quantified. The higher the level of analyte analogue in the solid phase, i.e., sticking to the ABM, the lower the level of analyte in the sample.

In a “sandwich assay”, both an insolubilized ABM, and a labeled ABM are employed. The analyte is captured by the insolubilized ABM and is tagged by the labeled ABM, forming a ternary complex. The reagents may be added to the sample in either order, or simultaneously. The ABMs may be the same or different. The amount of labeled ABM in the ternary complex is directly proportional to the amount of analyte in the sample.

The two embodiments described above are both heterogeneous assays. However, homogeneous assays are conceivable. The key is that the label be affected by whether or not the complex is formed.

Conjugation Methods

A label may be conjugated, directly or indirectly (e.g., through a labeled anti-ABM antibody), covalently (e.g., with SPDP) or noncovalently, to the ABM, to produce a diagnostic reagent. Similarly, the ABM may be conjugated to a solid phase support to form a solid phase (“capture”) diagnostic reagent.

Suitable supports include glass, polystyrene, polypropylene, polyethylene, dextran, nylon, amylases, natural and modified celluloses, polyacrylamides, agaroses, and magnetite. The nature of the carrier can be either soluble to some extent or insoluble for the purposes of the present invention.

The support material may have virtually any possible structural configuration so long as the coupled molecule is capable of binding to its target. Thus the support configuration may be spherical, as in a bead, or cylindrical, as in the inside surface of a test tube, or the external surface of a rod. Alternatively, the surface may be flat such as a sheet, test strip, etc.

Biological Assays

A biological assay measures or detects a biological response of a biological entity to a substance.

The biological entity may be a whole organism, an isolated organ or tissue, freshly isolated cells, an immortalized cell line, or a subcellular component (such as a membrane; this term should not be construed as including an isolated receptor). The entity may be, or may be derived from, an organism which occurs in nature, or which is modified in some way. Modifications may be genetic (including radiation and chemical mutants, and genetic engineering) or somatic (e.g., surgical, chemical, etc.) In the case of a multicellular entity, the modifications may affect some or all cells. The entity need not be the target organism, or a derivative thereof, if there is a reasonable correlation between bioassay activity in the assay entity and biological activity in the target organism.

The entity is placed in a particular environment, which may be more or less natural. For example, a culture medium may, but need not, contain serum or serum substitutes, and it may, but need not, include a support matrix of some kind, it may be still, or agitated. It may contain particular biological or chemical agents, or have particular physical parameters (e.g., temperature), that are intended to nourish or challenge the biological entity.

There must also be a detectable biological marker for the response. At the cellular level, the most common markers are cell survival and proliferation, cell behavior (clustering, motility), cell morphology (shape, color), and biochemical activity (overall DNA synthesis, overall protein synthesis, and specific metabolic activities, such as utilization of particular nutrients, e.g., consumption of oxygen, production of CO₂, production of organic acids, uptake or discharge of ions).

The direct signal produced by the biological marker may be transformed by a signal producing system into a different signal which is more observable, for example, a fluorescent or calorimetric signal.

The entity, environment, marker and signal producing system are chosen to achieve a clinically acceptable level of sensitivity, specificity and accuracy.

In some cases, the goal will be to identify substances which mediate the biological activity of a natural biological entity, and the assay is carried out directly with that entity. In other cases, the biological entity is used simply as a model of some more complex (or otherwise inconvenient to work with) biological entity. In that event, the model biological entity is used because activity in the model system is considered more predictive of activity in the ultimate natural biological entity than is simple binding activity in an in vitro system. The model entity is used instead of the ultimate entity because the former is more expensive or slower to work with, or because ethical considerations forbid working with the ultimate entity yet.

The model entity may be naturally occurring, if the model entity usefully models the ultimate entity under some conditions. Or it may be non-naturally occurring, with modifications that increase its resemblance to the ultimate entity.

Transgenic animals, such as transgenic mice, rats, and rabbits, have been found useful as model systems.

In cell-based model assays, where the biological activity is mediated by binding to a receptor (target protein), the receptor may be functionally connected to a signal (biological marker) producing system, which may be endogenous or exogenous to the cell.

There are a number of techniques of doing this.

“Zero-Hybrid” Systems

In these systems, the binding of a peptide to the target protein results in a screenable or selectable phenotypic change, without resort to fusing the target protein (or a ligand binding moiety thereof) to an endogenous protein. It may be that the target protein is endogenous to the host cell, or is substantially identical to an endogenous receptor so that it can take advantage of the latter's native signal transduction pathway. Or sufficient elements of the signal transduction pathway normally associated with the target protein may be engineered into the cell so that the cell signals binding to the target protein.

“One-Hybrid” Systems

In these systems, a chimera receptor, a hybrid of the target protein and an endogenous receptor, is used. The chimeric receptor has the ligand binding characteristics of the target protein and the signal transduction characteristics of the endogenous receptor. Thus, the normal signal transduction pathway of the endogenous receptor is subverted.

Preferably, the endogenous receptor is inactivated, or the conditions of the assay avoid activation of the endogenous receptor, to improve the signal-to-noise ratio.

See Fowlkes U.S. Pat. No. 5,789,184 for a yeast system.

Another type of “one-hybrid” system combines a peptide: DNA-binding domain fusion with an unfused target receptor that possesses an activation domain.

“Two-Hybrid” System

In a preferred embodiment, the cell-based assay is a two hybrid system. This term implies that the ligand is incorporated into a first hybrid protein, and the receptor into a second hybrid protein. The first hybrid also comprises component A of a signal generating system, and the second hybrid comprises component B of that system. Components A and B, by themselves, are insufficient to generate a signal. However, if the ligand binds the receptor, components A and B are brought into sufficiently close proximity so that they can cooperate to generate a signal.

Components A and B may naturally occur, or be substantially identical to moieties which naturally occur, as components of a single naturally occurring biomolecule, or they may naturally occur, or be substantially identical to moieties which naturally occur, as separate naturally occurring biomolecules which interact in nature.

Two-Hybrid System: Transcription Factor Type

In a preferred “two-hybrid” embodiment, one member of a peptide ligand:receptor binding pair is expressed as a fusion to a DNA-binding domain (DBD) from a transcription factor (this fusion protein is called the “bait”), and the other is expressed as a fusion to a transactivation domain (TAD) (this fusion protein is called the “fish”, the “prey”, or the “catch”). The transactivation domain should be complementary to the DNA-binding domain, i.e., it should interact with the latter so as to activate transcription of a specially designed reporter gene that carries a binding site for the DNA-binding domain. Naturally, the two fusion proteins must likewise be complementary.

This complementarity may be achieved by use of the complementary and separable DNA-binding and transcriptional activator domains of a single transcriptional activator protein, or one may use complementary domains derived from different proteins. The domains may be identical to the native domains, or mutants thereof. The assay members may be fused directly to the DBD or TAD, or fused through an intermediated linker.

The target DNA operator may be the native operator sequence, or a mutant operator. Mutations in the operator may be coordinated with mutations in the DBD and the TAD. An example of a suitable transcription activation system is one comprising the DNA-binding domain from the bacterial repressor LexA and the activation domain from the yeast transcription factor Gal4, with the reporter gene operably linked to the LexA operator.

It is not necessary to employ the intact target receptor; just the ligand-binding moiety is sufficient.

The two fusion proteins may be expressed from the same or different vectors. Likewise, the activatable reporter gene may be expressed from the same vector as either fusion protein (or both proteins), or from a third vector.

Potential DNA-binding domains include Gal4, LexA, and mutant domains substantially identical to the above.

Potential activation domains include E. coli B42, Gal4 activation domain II, and HSV VP16, and mutant domains substantially identical to the above.

Potential operators include the native operators for the desired activation domain, and mutant domains substantially identical to the native operator.

The fusion proteins may comprise nuclear localization signals.

The assay system will include a signal producing system, too. The first element of this system is a reporter gene operably linked to an operator responsive to the DBD and TAD of choice. The expression of this reporter gene will result, directly or indirectly, in a selectable or screenable phenotype (the signal). The signal producing system may include, besides the reporter gene, additional genetic or biochemical elements which cooperate in the production of the signal. Such an element could be, for example, a selective agent in the cell growth medium. There may be more than one signal producing system, and the system may include more than one reporter gene.

The sensitivity of the system may be adjusted by, e.g., use of competitive inhibitors of any step in the activation or signal production process, increasing or decreasing the number of operators, using a stronger or weaker DBD or TAD, etc.

When the signal is the death or survival of the cell in question, or proliferation or nonproliferation of the cell in question, the assay is said to be a selection. When the signal merely results in a detectable phenotype by which the signaling cell may be differentiated from the same cell in a nonsignaling state (either way being a living cell), the assay is a screen. However, the term “screening assay” may be used in a-broader sense to include a selection. When the narrower sense is intended, we will use the term “nonselective screen”.

Various screening and selection systems are discussed in Ladner, U.S. Pat. No. 5,198,346.

Screening and selection may be for or against the peptide: target protein or compound:target protein interaction.

Preferred assay cells are microbial (bacterial, yeast, algal, protozooal), invertebrate, vertebrate (esp. mammalian, particularly human). The best developed two-hybrid assays are yeast and mammalian systems.

Normally, two hybrid assays are used to determine whether a protein X and a protein Y interact, by virtue of their ability to reconstitute the interaction of the DBD and the TAD. However, augmented two-hybrid assays have been used to detect interactions that depend on a third, non-protein ligand.

For more guidance on two-hybrid assays, see Brent and Finley, Jr., Ann. Rev. Genet., 31:663-704 (1997); Fremont-Racine, et al., Nature Genetics, 277-281 (16 Jul. 1997); Allen, et al., TIBS, 511-16 (December 1995); LeCrenier, et al., BioEssays, 20:1-6 (1998); Xu, et al., Proc. Nat. Acad. sci. (USA), 94:12473-8 (November 1992); Esotak, et al., Mol. Cell. Biol., 15:5820-9 (1995); Yang, et al., Nucleic Acids Res., 23:1152-6 (1995); Bendixen, et al., Nucleic Acids Res., 22:1778-9 (1994); Fuller, et al., BioTechniques, 25:85-92 (July 1998); Cohen, et al., PNAS (USA) 95:14272-7 (1998); Kolonin and Finley, Jr., PNAS (USA) 95:14266-71 (1998). See also Vasavada, et al., PNAS (USA), 88:10686-90 (1991) (contingent replication assay), and Rehrauer, et al., J. Biol. Chem., 271:23865-73 91996) (LexA repressor cleavage assay).

Two-Hybrid Systems: Reporter Enzyme Type

In another embodiment, the components A and B reconstitute an enzyme which is not a transcription factor.

As in the last example, the effect of the reconstitution of the enzyme is a phenotypic change which may be a screenable change, a selectable change, or both.

In vivo Diagnostic Uses

Radio-labeled ABM may be administered to the human or animal subject. Administration is typically by injection, e.g., intravenous or arterial or other means of administration in a quantity sufficient to permit subsequent dynamic and/or static imaging -using suitable radio-detecting devices. The dosage is the smallest amount capable of providing a diagnostically effective image, and may be determined by means conventional in the art, using known radio-imaging agents as a guide.

Typically, the imaging is carried out on the whole body of the subject, or on that portion of the body or organ relevant to the condition or disease under study. The amount of radio-labeled ABM accumulated at a given point in time in relevant target organs can then be quantified.

A particularly suitable radio-detecting device is a scintillation camera, such as a gamma camera. A scintillation camera is a stationary device that can be used to image distribution of radio-labeled ABM. The detection device in the camera senses the radioactive decay, the distribution of which can be recorded. Data produced by the imaging system can be digitized. The digitized information can be analyzed over time discontinuously or continuously. The digitized data can be processed to produce images, called frames, of the pattern of uptake of the radio-labeled ABM in the target organ at a discrete point in time. In most continuous (dynamic) studies, quantitative data is obtained by observing changes in distributions of radioactive decay in target organs over time. In other words, a time-activity analysis of the data will illustrate uptake through clearance of the radio-labeled binding protein by the target organs with time.

Various factors should be taken into consideration in selecting an appropriate radioisotope. The radioisotope must be selected with a view to obtaining good quality resolution upon imaging, should be safe for diagnostic use in humans and animals, and should preferably have a short physical half-life so as to decrease the amount of radiation received by the body. The radioisotope used should preferably be pharmacologically inert, and, in the quantities administered, should not have any substantial physiological effect.

The ABM may be radio-labeled with different isotopes of iodine, for example ¹²³I, ¹²⁵I, or ¹³³I (see for example, U.S. Pat. No. 4,609,725). The extent of radio-labeling must, however be monitored, since it will affect the calculations made based on the imaging results (i.e. a diiodinated ABM will result in twice the radiation count of a similar monoiodinated ABM over the same time frame).

In applications to human subjects, it may be desirable to use radioisotopes other than ¹²⁵I for labeling in order to decrease the total dosimetry exposure of the human body and to optimize the detectability of the labeled molecule (though this radioisotope can be used if circumstances require). Ready availability for clinical use is also a factor. Accordingly, for human applications, preferred radio-labels are for example, ^{99m}Tc, ⁶⁷Ga, ⁶⁸Ga, ⁹⁰Y, ¹¹¹In, ^{123m}In, ¹²³I, ¹⁸⁶Re, ¹⁸⁸Re or ²¹¹At.

The radio-labeled ABM may be prepared by various methods. These include radio-halogenation by the chloramine—T method or the lactoperoxidase method and subsequent purification by HPLC (high pressure liquid chromatography), for example as described by J. Gutkowska et al in “Endocrinology and Metabolism Clinics of America: (1987) 16 (1): 183. Other known methods of radio-labeling can be used, such as IODOBEADS™.

There are a number of different methods of delivering the radio-labeled ABM to the end-user. It may be administered by any means that enables the active agent to reach the agent's site of action in the body of a mammal. Because proteins are subject to being digested when administered orally, parenteral administration, i.e., intravenous, subcutaneous, intramuscular, would ordinarily be used to optimize absorption of an ABM, such as an antibody, which is a protein.

### EXAMPLES

### Example 1

Differentially expressed mouse genes, and corresponding human genes/proteins, were identified as described in this Example, and compiled into Master Table 1.

Animal Models Upon separation from their mothers (weaning), C57Bl/6J mice (i.e., C577Bl/6 mice developed by Jackson Labs) were placed on a normal diet (PMI Nutritiori International Inc., Brentwood, Mo., Prolab RMH3000). Mice were sacrificed at an average of 35, 49, 56, 77, 118, 133, 207, 403, 558 and 725 days of age.

RNA Isolation.

Total RNA was isolated from livers using the RNA STAT-60 Total PHA/mRNA Isolation Reagent according to the manufacturer's instructions (Tel-Test, Friendswood, Tex.).

Sample Quantification and Quality Assessment

Total RNA was quantified and assessed for quality on a Bioanalyzer RNA 6000 Nano chip (Agilent). Each chip contained an interconnected set of gel-filled channels that allowed for molecular sieving of nucleic acids. Pin-electrodes in the chip were used to create electrokinetic forces capable of driving molecules through these micro-channels to perform electrophoretic separations. Ribosomal peaks were measured by fluorescence signal and displayed in an electropherogram. A successful total RNA sample featured 2 distinct ribosomal peaks (18S and 28S rRNA).

Biotinylated cRNA Hybridization Target.

Total PNA was prepared for use as a hybridization target as described in the manufacturer's instructions for CodeLink Expression Bioarrays(TM) (Amersham Biosciences). The CodeLink Expression Bioarrays utilize nucleic acid hybridization of a biotin-labeled complementary RNA(cRNA) target with DNA oligonucleotide probes attached to a gel matrix.

The biotin-labeled cRNA target is prepared by a linear amplification method. Poly (A)+RNA (within the total RNA population) is primed for reverse transcription by a DNA oligonucleotide containing a T7 RNA polymerase promoter 5′ to a (dT) 24 sequence. After second-strand cDNA synthesis, the cDNA serves as the template in an in vitro transcription (IVT) reaction to produce the target cRNA. The IVT is performed in the presence of biotinylated nucleotides to label the target cRNA. This procedure results in a 50-200 fold linear amplification of the input poly (A)+RNA.

Hybridization Probes.

The oligonucleotide probes were provided by the Codelink Uniset Mouse I Bioarray (Amersham, product code 300013). Amine-terminated oligonucleotide probes are attached to a three-dimensional polyacrylamide gel matrix. There are 10,000 oligonucleotide probes, each specific to a well-characterized mouse gene. Each mouse gene is representative of a unique gene cluster from the fourth quarter 2001 Genbank Unigene build. There are also 500 control probes.

The sequences of the probes are proprietary to Amersham. However, for each probe, Amersham identifies the corresponding mouse gene by NCBI accession number, OGS, LocusLink, Unigene Cluster ID, and description (name). This information should be available from Amersham. In the case of the differentially expressed probes, this information is duplicated in master table 1. Fox the complete list, see

http://www4.amershambiosciences.com/aptrix/upp01077.nsf/Cont ent/codelink_literature

Under “Gene Lists”, select “Uniset Mouse I”, and a gene list, in Excel format, can be downloaded.

Hybridization

Using the cRNA target, the hybridization reaction mixture is prepared and loaded into array chambers for bioarray processing as set forth in the manufacturer's instructions for CodeLink Gene Expression Bioarrays™ (Amerhsam Biosciences). Each sample is hybridized to an individual microarray. Hybridization is at 37° C. The hybridization buffer is prepared as set forth in the Motorola instructions. Hybridization to the microarray is detected with an avidinated fluorescent reagent, Streptavidin-Alexa Fluor®647 (Amersham).

Mouse Gene Expression Analysis

Processed arrays were scanned using a GenePix 4000B Microarray Scanner (Axon Instruments, Inc.); array images were acquired using the Amersham CodeLink™ Analysis Software (Release 2.2). The Amersham CodeLink™ Analysis Software gives an integrated optical density (IOD) value for every spot; a unique background value for that spot is subtracted, resulting in “raw” data points. Individual chips are then normalized by the Amersham Codelink™ software according to the median raw intensity for all 10,000 genes. A negative control threshold (0.2) was also calculated according to the control probes. A significant difference in expression between samples was defined as a minimum of 2-fold change in expression values. Genes with expression values below the negative control threshold were eliminated from the analysis and then the expression data was analyzed to identify genes whose expression levels changed significantly with respect to age.

The list of genes in the tables is a combination of two analyses. Samples of average age 35, 49, 77 and 133 days were compared pair-wise in all possible combinations (6 comparisons) and genes showing differences in expression greater than 2-fold were listed in the table. (The 56 day data was not included in the comparisons.) The remaining samples were divided into three groups (118 days (2 mice): young; 207 and 403 (4 mice) averaged together: medium; 558 and 725 (4 mice) averaged together: old), the three groups were compared in all possible pair-wise combinations (3 comparisons) and genes showing differences in expression greater than 2-fold were added to the table.

Database Searches Nucleotide sequences and predicted amino acid sequences were compared to public domain databases using the Blast 2.0 program (National Center for Biotechnology Information, National Institutes of Health). Nucleotide sequences were displayed using ABI prism Edit View 1.0.1 (PE Applied Biosystems, Foster City, Calif.).

Nucleotide database searches were conducted with the never submitted to an archival database but is available in the literature. A small number of sequences are provided through collaboration; the underlying primary sequence data is available in GenBank, but may not be available in any one GenBank record. RefSeq sequences are not submitted primary sequences. RefSeq records are owned by NCBI and therefore can be updated as needed to maintain current annotation or to incorporate additional sequence information.” See also http://www.ncbi.nlm.nih.gov/LocusLink/refseg.html

It will be appreciated by those in the art that the exact results of a database search will change from day to day, as new sequences are added. Also, if you query with a longer version of the original sequence, the results will change. The results given here were obtained at one time and no guarantee is made that the exact same hits would be obtained in a search on the filing date. However, if an alignment between a particular query sequence and a particular database sequence is discussed, that alignment should not change (if the parameters and sequences remain unchanged).

Northern Analysis.

Northern analysis may be used to confirm the results. Favorable and unfavorable genes, identified as described above, or fragments thereof, will be used as probes in Northern hybridization analyses to confirm their differential expression. Total RNA isolated from subject mice will be resolved by agarose gel electrophoresis through a 1% agarose, 1% formaldehyde denaturing gel, transferred to positively charged nylon membrane, and hybridized to a probe labeled with [32P] dCTP that was generated from the aforementioned gene or fragment using the Random Primed DNA Labeling Kit (Roche, Palo Alto, Calif.), or to a probe labeled with digoxygenin according to the manufacturer's instructions (Roche, Palo Alto, Calif.).

Real-Time RNA Analysis.

Real-time RNA analysis may also be used for confirmation. For “real-time” RNA analysis, RNA will be converted to cDNA and then probed with gene-specific primers made for each clone. “Real-time” incorporation of fluorescent dye will be measured to determine the amount of specific transcript present in each sample. Sample differences (older vs. younger) of 2-fold or greater (in either direction) will be considered differentially expressed. Confirmation using several independent animals is desirable.

In situ Hybridization

Another form of confirmation may be provided by nonisotopic in situ hybridizations (ISH) on selected human (obtained by Tissue Informatics) and mouse tissues using cRNA probes generated from mouse genes found to be up- or down-regulated during aging. In situ hybridizations may also be performed on mouse tissues using cRNA probes generated from differentially expressed DNAs. These cRNA's will hybridize to their corresponding messenger RNA's present in cells and will provide information regarding the particular cell types within a tissue that is expressing the particular gene as well as the relative level of gene, expression. The cRNA probes may be generated by in vitro transcription of template cDNA by Sp6 or T7 RNA polymerase in the presence of digoxigenin-11-UTP (Roche Molecular Biochemicals, Mannheim, Germany; Pardue, M. L. 1985. In: In situ hybridization, Nucleic acid hybridization, a practical approach: IRL Press, Oxford, 179-202).

Transgenic Animals.

Transgenic expression may be used to confirm the results. In one embodiment, a mouse is engineered to overexpress the favorable or unfavorable mouse gene in question. In another embodiment, a mouse is engineered to express the corresponding favorable or unfavorable human gene. In a third embodiment, a nonhuman animal other than a mouse, such as a rat, rabbit, goat, sheep or pig, is engineered to express the favorable or unfavorable mouse or human gene.

Hyperquantitative Tissue Analysis

In addition to gene expression analysis the tissue sections can also be analyzed using TissueInformatics, Inc's TissueAnalytics™ software. A single representative section may be cut from each tissue block, placed on a slide, and stained with H&E. Digital images of each slide may be acquired using an research microscope and digital camera (Olympus E600 microscope and Sony DKC-ST5). These images were acquired at 20x magnification with a resolution of 0.64 mm/pixel. A hyperquantitative analysis may be performed on the resulting images: First a digital image analysis can identify and annotate structural objects in a tissue using machine vision. These objects, that are constituents of the tissue, can be annotated because they are visually identifiable and have a biological meaning. (By way of example, for liver, the constituents can be, e.g., hepatocytes, sinusoids, vacuoles.) Subsequently a quantification of these structures regarding their geometric properties like area or stain intensities and their relationship to the field of view or per unit area in terms of a % coverage may be performed. Features or parameters for hyper-quantification are specific fox each tissue, and may also include relations between features, measures of overall heterogeneity, including orientation, relative locations, and textures.

Correlation Analysis

Mathematical statistics provides a rich set of additional tools to analyze time resolved data sets of hyper-quantitative and gene expression profiles for similarities, including rank correlation, the calculation of regression and correlations coefficients, and clustering. Continuous functions may also be fitted through the data points of individual gene and tissue feature data. Relation between gene expression and hyper-quantitative tissue data may be linear or non-linear, in synchronous or asynchronous arrangements.

Introduction to Master Tables

The master tables reflect applicants' analysis of the gene chip data.

For each probe corresponding to a differentially expressed mouse gene, Master Table 1 identifies

Col. 1: The mouse gene (upper) and mouse protein (lower) database accession #s.

Col. 2: The corresponding mouse Unigene Cluster, as of the 4^{th} Quarter 2001 build.

Col. 3: The behavior (differential expression) observed for the mouse gene. This column identifies the gene as favorable(F) or unfavorable (U) on the basis of its differential behavior in the comparisons (older vs. younger). As more than one older vs. younger comparison is made, only the result of the comparison yielding the greatest differential is listed. In the case of a gene with mixed behavior, both the result of the comparison yielding the greatest favorable differential and the result of the comparison yielding the greatest unfavorable differential are listed. If the value is followed by a parenthetical of the form “(X to Y)”, it means that the differential value is the ratio when the absolute value for X weeks was compared to the absolute value for Y weeks, with the ratio being taken as greater-to-lesser.

One possible way of characterizing the degree of differential expression for a particular comparison would be to take the ratio of older to younger. If that ratio is at least 2:1, the behavior is considered unfavorable, and if it is not more than 0.5:1, it is unfavorable.

Use of an older/younger ratio is awkward when one wants to compare the degree of differential expression without regard to the direction of change. Consequently, in the Master Table, the numerical value is the ratio of the greater value to the lesser value. If this ratio is at least two fold, the degree of differential expression is considered significant.

In some of the related applications cited above, and perhaps occasionally in this application, a ratio may be given as a negative number. This does not have its usual mathematical meaning; it is merely a flag that in the comparison, the older value was less than the younger one, i.e., the gene was favorable. For the purpose of applying the teachings of the specification concerning desired ratios, any negative value should be converted to a positive one by taking its absolute value.

Col. 4: A related human protein, identified by its database accession number. Usually, several such proteins are identified relative to each mouse gene. These proteins have been identified by BLAST searches, as explained in cols. 6-8.

Col. 5: The name of the related human protein.

Col. 6: The score (in bits) for the alignment performed by the BLAST program.

Col. 7: The E-value for the alignment performed by the BLAST program. It is worth noting that Unigene considers a Blastx E Value of less than le-6 to be a “match” to the reference sequence of a cluster.

Unless otherwise indicated, the bit score and E-value for the alignment is with respect to the alignment of the mouse DNA of col. 1 to the human protein of col. 4 by BlastX, according to the default parameters.

Master Table 1 is divided into two or three subtables on the basis of the Behavior” in col. 3. If a gene has at least one favorable behavior, and no unfavorable ones, it is put into Subtable 1A. In the opposite case, it is put into Subtable 1B. If any of the genes has mixed behavior, then Master Table 1 will include Subtable 1C for such genes.

Master Table 2 has just three columns.

Col. 1: Mouse gene.

Col. 2: behavior. Same as col. 3 in Master table 1.

Col. 3: Human protein classes. Based on the related human proteins defined in Master Table 1, Master Table 2 generalizes, if possible as to classes of human proteins which are expected to have similar behavior. For a given mouse gene, several human protein classes may be listed because of the diversity of the human proteins found to be related. In some cases, the stated human protein classes may be hierarchial, e.g., one may be a subset of another. In other cases, the stated classes may be non-overlapping but related. And in yet other cases, the stated classes may be non-overlapping and unrelated. Combinations of the above are also possible.

In addition to the classes stated, the corresponding human gene clusters are also of interest. These may be obtained in a number of ways. First, one may search on Unigene (http://www.ncbi.nlm.nih.gov/entrez/query.fcgi?db=unigene) for the identified human protein. Review the “hits” (each of which is a Unigene record) for those prefixed by “Hs.” Secondly, one may access the Unigene record for the mouse gene cluster (which is given in Master Table 1), and then click on “Homologene”. This will bring up a new page which includes the section “Possible Homologous Genes”. One of the entries should be a Homo sapiens gene (considered by Unigene to be the most related human gene); click on its Unigene record link.

Additional information of interest may be accessed by searching with the mouse gene accession # in the Mouse Gene Informatics database, at http://www.informatics.jax.org/.

The related applications may contain reference to “2-16 week old mice”. In the anti-diabetes series of applications, 3 week mice were put on a diet to induce obesity, hyperinsulinemia and diabetes. The 2-16 week old mice were more accurately described as mice who had been on that diet for 2-16 weeks, i.e., they were actually 5-19 weeks (35-133 days) old. Even some of the anti-aging series of applications made reference to 2-16 week old mice, even though the mice were in fact 5-19 weeks (35-133 days) old.

LENGTHY TABLE REFERENCED HERE
US20070111933A1-20070517-T00001
Please refer to the end of the specification for access instructions.

LENGTHY TABLE REFERENCED HERE
US20070111933A1-20070517-T00002
Please refer to the end of the specification for access instructions.

LENGTHY TABLE REFERENCED HERE
US20070111933A1-20070517-T00003
Please refer to the end of the specification for access instructions.

LENGTHY TABLE REFERENCED HERE
US20070111933A1-20070517-T00004
Please refer to the end of the specification for access instructions.

LENGTHY TABLE REFERENCED HERE
US20070111933A1-20070517-T00005
Please refer to the end of the specification for access instructions.

LENGTHY TABLE REFERENCED HERE
US20070111933A1-20070517-T00006
Please refer to the end of the specification for access instructions.

Master Tables 101-199

In the related applications set forth at the beginning of the specification, we have looked at differential expression of genes in various organs and tissue with respect to (1) aging, (2) hyperinsulinemia and/or type II diabetes. Master Tables 101-199 (note that some of these table numbers are reserved for future use) tabulate those mouse genes which appear both in Master Table 1 of this application, and in the corresponding table of at least one of the related applications.

The following human proteins are considered to be of particular interest:
Human proteins corresponding to mouse genes listed as favorable both in Master Table I and in at least one of Master Tables 101-199, which are not listed as unfavorable in any of Master Tables 101-199; and

Human proteins corresponding to mouse genes listed as unfavorable both in Master Table 1 and in at least one of Master Tables 101-199, which are not listed as favorable in any of Master Tables 101-199.

MASTER TABLE 101
Genes Differentially Expressed With Respect to Age in Both
Liver and Muscle

| | | Liver | Muscle |
|---|---|---|---|
| Mouse | | Aging | Aging |
| Gene | Mouse Description | Behavior | Behavior |
| AF281045 | Mus musculus 2-5A-dependent RNase L mRNA, | U: 4.86 | U: +2.12 |
| | complete cds | (5 to 11) | |
| AF316872 | Mus musculus protein kinase BRPK mRNA, | U: 2.16 | U: +2.26 |
| | complete cds | (Y to M) | F: 3.65 |
| AK015750 | AK015750 Mus musculus adult male testis | U: 2.56 | U: +7.39 |
| | cDNA, RIKEN full-length enriched library, | (Y to O) | |
| | clone: 4930511F10: sulfotransferase, estrogen | | |
| | preferring, full insert sequence | | |
| AK018226 | Mus musculus adult male medulla oblongata | U: 4.01 | F: 2.35 |
| | cDNA, RIKEN full-length enriched library, | (5 to 19) | |
| | clone: 6330533H24, full insert sequence | | |
| J04694 | MUSCOL1A4A Mus musculus alpla-1 type IV | F: 2.05 | F: 6.66 |
| | collagen (Col4a-1) mRNA, complete cds | (5 to 11) | |
| NM_007702 | Mus musculus cell death-inducing DNA | U: 52.77 | U: +1.88 |
| | fragmentation factor, alpha subunit-like | (Y to O) | |
| | effector A (Cidea), mRNA | | |
| NM_007952 | Mus musculus glucose regulated protein, 58 kDa | F: 2.65 | F: 2.59 |
| | (Grp58), mRNA | (5 to 19) | |
| NM_008161 | Mus musculus glutathione peroxidase 3 | U: 3.13 | U: +2.43 |
| | (Gpx3), mRNA | (Y to O) | |
| NM_008524 | Mus musculus lumican (Lum), mRNA | F: 2.41 | F: 2.01 |
| | | (5 to 19) | |
| NM_009075 | Mus musculus ribose 5-phosphate isomerase A | U: 2.09 | F: 2.48 |
| | (Rpia), mRNA | (Y to O) | |
| NM_009242 | Mus musculus secreted acidic cysteine rich | F: 2.73 | F: 4.66 |
| | glycoprotein (Sparc), mRNA | (5 to 19) | |
| NM_009381 | Mus musculus thyroid hormone responsive | U: 5.69 | F: 2.18 |
| | SPOT14 homolog (Rattus) (Thrsp), mRNA | (Y to O) | |
| NM_010238 | Mus musculus bromodomain-containing 2 | F: 2.33 | F: 2.27 |
| | (Brd2), mRNA | (7 to 19) | |
| NM_010917 | Mus musculus nidogen 1 (Nid1), mRNA | F: 2.3 | F: 2.54 |
| | | (5 to 11) | |
| NM_011579 | Mus musculus T-cell specific GTPase (Tgtp), | F: 2.1 | U: +2.72 |
| | mRNA | (5 to 19) | |
| NM_016906 | Mus musculus SEC61, alpha subunit (S. cerevisiae) | F: 2.37 | U: +2.79 |
| | (Sec61a), mRNA | (5 to 19) | F: 3.89 |
| NM_019750 | Mus musculus N-acetyltransferase 6 (Nat6), | F: 2.02 | F: 2.55 |
| | mRNA | (5 to 19) | |
| NM_019824 | Mus musculus actin related protein 2/3 | F: 5.75 | U: +2.14 |
| | complex, subunit 3 (21 kDa) (Arpc3), mRNA | (7 to 19) | |
| NM_021301 | Mus musculus solute carrier family 15 | F: 3.08 | F: 2.35 |
| | (H+/peptide transporter), member 2 | (Y to M) | |
| | (Slc15a2), mRNA | | |
| NM_022434 | Mus musculus cytochrome P450, subfamily IVF, | F: 2.19 | U: +2.12 |
| | polypeptide 14 (leukotriene B4 omega | (5 to 19) | |
| | hydroxylase) (Cyp4f14), mRNA | | |
| NM_023184 | Mus musculus Kruppel-like factor 15 (Klf15), | F: 2.87 | U: +2.85 |
| | mRNA | (5 to 11) | F: 4.85 |
| NM_026189 | Mus musculus RIKEN cDNA 2310005P05 gene | U: 2.29 | U: +2.14 |
| | (2310005P05Rik), mRNA | (5 to 11) | |
| NM_026346 | Mus musculus RIKEN cDNA 4833442G10 gene | F: 3.64 | U: +6.12 |
| | (4833442G10Rik), mRNA | (Y to O) | |
| U89415 | MMU89415 Mus musculus strain BALB/c | F: 2.73 | U: +2.02 |
| | elongation factor 2 mRNA, partial cds | (5 to 19) | F: 2.92 |

TABLE 102
Mouse Genes Differentially Expressed in Liver with respect
to both Diabetes/Hyperinsulinemia and Aging

| | | Behavior | Behavior |
|---|---|---|---|
| Gene | Description | Diabetes | Aging |
| AF047725 | Mus musculus CYP2C38 (Cyp2c38) mRNA, partial | F: (IR-D) | U: 2.28 |
| | cds | 2.06 | (5 to 11) |
| | | U: (C-D) | |
| | | 2.35 | |
| AF127033 | Mus musculus fatty acid synthase mRNA, | F: (IR-D) | U: 2.97 |
| | complete cds | 2.1 | (Y to O) |
| AF294617 | Mus musculus inducible | F: (C-IR) | F 2.69 |
| | 6-phosphofructo-2-kinase mRNA, complete cds | 2.63 | (5 to 7) |
| AF385682 | Mus musculus ETL1 mRNA, complete cds | F: (C-IR) | F 2.03 |
| | | 2.04, | (7 to 11) |
| | | U: (IR-D) | |
| | | 2.02 | |
| AK002693 | Mus musculus adult male kidney cDNA, RIKEN | U: (C-IR) | U: 2.55 |
| | full-length enriched library, | 2.04 | (Y to O) |
| | clone: 0610030A14: related to COSMID W01A11, | | |
| | full insert sequence | | |
| AK002979 | Mus musculus adult male brain cDNA, RIKEN | F: (C-IR) | U: 2.67 |
| | full-length enriched library, | 2.14, | (5 to 19) |
| | clone: 0710001P07: homolog to D1 DOPAMINE | F: (C-D) | |
| | RECEPTOR INTERACTING PROTEIN CALCYON, full | 2.15 | |
| | insert sequence | | |
| AK002979 | Mus musculus adult male brain cDNA, RIKEN | F: (C-IR) | U: 2.67 |
| | full-length enriched library, | 2.14, | (5 to 19) |
| | clone: 0710001P07: homolog to D1 DOPAMINE | F: (C-D) | |
| | RECEPTOR INTERACTING PROTEIN CALCYON, full | 2.15 | |
| | insert sequence | | |
| AK005274 | Mus musculus adult male cerebellum cDNA, | U: (C-IR) | F 3.89 |
| | RIKEN full-length enriched library, | 2.22, | (5 to 7) |
| | clone: 1500017E18: homolog to | U: (C-D) | |
| | HYDROXYACYLGLUTATHIONE HYDROLASE (EC | 2.15 | |
| | 3.1.2.6) (GLYOXALASE II) (GLX II), full | | |
| | insert sequence | | |
| AK005535 | Mus musculus adult female placenta cDNA, | F: (C-IR) | F 3.25 |
| | RIKEN full-length enriched library, | 2.06, | (Y to M) |
| | clone: 1600025H15: homolog to CDNA FLJ20327 | F: (C-D) | |
| | FIS, CLONE HEP10012, full insert sequence | 2.16 | |
| AK006096 | AK006096 Mus musculus adult male testis | U: (C-IR) | U: 4.75 |
| | cDNA, RIKEN full-length enriched library, | 2.24 | (Y to O) |
| | clone: 1700018O18: hypothetical protein, full | | |
| | insert sequence | | |
| AK007264 | Mus musculus adult male testis cDNA, RIKEN | F: (C-IR) | F 2.04 |
| | full-length enriched library, | 2.95, | (5 to 19) |
| | clone: 1700124F02: homolog to | U: (IR-D) | |
| | WUGSC: H_NH0335J18.1 PROTEIN, full insert | 2.34 | |
| | sequence | | |
| AK007293 | Mus musculus adult male testis cDNA, RIKEN | U: (C-D) | U: 3.56 |
| | full-length enriched library, | 2.19, | (5 to 11) |
| | clone: 1700126L06: unclassifiable, full insert | U: (IR-D) | |
| | sequence | 2.62 | |
| AK009563 | Mus musculus adult male tongue cDNA, RIKEN | F: (C-IR) | F 2.1 |
| | full-length enriched library, | 2.33 | (5 to 19) |
| | clone: 2310032D16, full insert sequence | | |
| AK018226 | Mus musculus adult male medulla oblongata | F: (C-IR) | U: 4.01 |
| | cDNA, RIKEN full-length enriched library, | 2.53, | (5 to 19) |
| | clone: 6330533H24, full insert sequence | F: (C-D) | |
| | | 2.4 | |
| M12571 | MUSHSP68A Mouse heat shock protein (hsp68) | U: (C-IR) | F 2.73 |
| | mRNA, clone MHS243, partial cds | 3.58 | (Y to M) |
| M12573 | MUSHSP68C Mouse heat shock protein (hsp68) | U: (C-D) | F 2.07 |
| | mRNA, clone MHS214, partial cds | 2.94 | (5 to 19) |
| M62766 | MUSHMGCOA Mouse HMG-CoA reductase mRNA, 3′ | U: (C-IR) | U: 2.16 |
| | end | 2.02 | (Y to M) |
| M63245 | MUSALASH Mus musculus amino levulinate | U: (C-IR) | F3.98 |
| | synthase (ALAS-H) mRNA, 3′ end | 3.05 | (5 to 19) |
| NM_007468 | Mus musculus apolipoprotein A-IV (Apoa4), | U (C-IR) | F 2.22 |
| | mRNA | 2.98, U | (7 to 11) |
| | | (C-D) | |
| | | 2.42, U | |
| | | (IR-D) | |
| | | 2.16 | |
| NM_007472 | Mus musculus aquaporin 1 (Aqp1), mRNA | F: (C-IR) | F 2.04 |
| | | 2.17, | (7 to 11) |
| | | U: (IR-D) | |
| | | 2.38 | |
| NM_007489 | Mus musculus aryl hydrocarbon receptor | F: (C-D) − 2.13 | F 2.22 |
| | nuclear translocator-like (Arntl), mRNA | | (7 to 11) |
| NM_007643 | Mus musculus CD36 antigen (Cd36), mRNA | F: (C-IR) | U: 3.57 |
| | | 3.03, | (Y to O) |
| | | U: (C-D) | |
| | | 2.05, | |
| | | U: (IR-D) | |
| | | 3.33 | |
| NM_007702 | Mus musculus cell death-inducing DNA | U: (C-D) + 4.7 | U: 52.77 |
| | fragmentation factor, alpha subunit-like | | (Y to O) |
| | effector A (Cidea), mRNA | | |
| NM_007706 | Mus musculus cytokine inducible | F: (C-D) | F4.4 |
| | SH2-containing protein 2 (Cish2), mRNA | 2.51 | (Y to M) |
| NM_007760 | Mus musculus carnitine acetyltransferase | U: (C-IR) | U: 2.41 |
| | (Crat), mRNA | 2.57, | (5 to 7) |
| | | U: (C-D) | |
| | | 2.16 | |
| NM_007809 | Mus musculus cytochrome P450, 17 (Cyp17), | U: (C-IR) | U: 3.27 |
| | mRNA | 3.41, | (Y to O) |
| | | U: (C-D) | |
| | | 3.69 | |
| NM_007811 | Mus musculus cytochrome P450, 26, retinoic | F: (C-IR) | F 2.08 |
| | acid (Cyp26), mRNA | 17.03, | (5 to 11) |
| | | F: (C-D) | |
| | | 3.81 | |
| NM_007822 | Mus musculus cytochrome P450, 4a14 | U: (C-IR) | U: 18.8 |
| | (Cyp4a14), mRNA | 24.5, | (5 to 7) |
| | | F: (C-D) | |
| | | 5.06, | |
| | | F: (IR-D) | |
| | | 7.06 | |
| NM_007824 | Mus musculus cytochrome P450, 7a1 (Cyp7a1), | F: (C-IR) | U: 2.47 |
| | mRNA | 2.14, | (Y to M) |
| | | F: (C-D) | |
| | | 3.09 | |
| NM_007825 | Mus musculus cytochrome P450, 7b1 (Cyp7b1), | F: (C-IR) | F 2.22 |
| | mRNA | 6.41, | (5 to 19) |
| | | U: (IR-D) | |
| | | 5.83 | |
| NM_007860 | Mus musculus deiodinase, iodothyronine, type | U: (C-IR) | F 2.06 |
| | I (Dio1), mRNA | 2.84, | (7 to 19) |
| | | U: (C-D) | |
| | | 2.06 | |
| NM_007912 | Mus musculus epidermal growth factor | F: (C-IR) | F 2.21 |
| | receptor (Egfr), mRNA | 2.09, | (5 to 19) |
| | | F: (C-D) | |
| | | 2.69 | |
| NM_008039 | Mus musculus formyl peptide receptor, | F: (C-D) − 2.4 | F 2.04 |
| | related sequence 2 (Fpr-rs2), mRNA | | (Y to O) |
| NM_008061 | Mus musculus glucose-6-phosphatase, | F: (C-IR) | F 2.75 |
| | catalytic (G6pc), mRNA | 2.28, | (5 to 11) |
| | | F: (C-D) | |
| | | 2.14 | |
| NM_008182 | Mus musculus glutathione S-transferase, | F: (C-IR) | U: 5.76 |
| | alpha 2 (Yc2) (Gsta2), mRNA | 9.17, | (5 to 19) |
| | | F: (C-D) | |
| | | 5.68 | |
| NM_008245 | Mus musculus hematopoietically expressed | F: (C-D) | F 2.2 |
| | homeobox (Hhex), mRNA | 2.62, | (7 to 19) |
| | | U: (IR-D) | |
| | | 2.05 | |
| NM_008295 | Mus musculus hydroxysteroid dehydrogenase-5, | F: (C-IR) | F 2.25 |
| | delta<5>-3-beta (Hsd3b5), mRNA | 2.43, | (Y to O) |
| | | F: (C-D) | |
| | | 5.64, | |
| | | F: (IR-D) | |
| | | 2.32 | |
| NM_008341 | Mus musculus insulin-like growth factor | F: (C-IR) | F13.28 |
| | binding protein 1 (Igfbp1), mRNA | 3.37, | (5 to 11) |
| | | F: (C-D) | |
| | | 3.47, | |
| | | F: (IR-D) | |
| | | 2.63 | |
| NM_008361 | Mus musculus interleukin 1 beta (Il1b), mRNA | F: (C-IR) | U: 3.05 |
| | | 2.65, | (5 to 7) |
| | | F: (C-D) | |
| | | 2.03 | |
| NM_008362 | Mus musculus interleukin 1 receptor, type I | U: (C-IR) | F 2.26 |
| | (Il1r1), mRNA | 2.59, | (5 to 19) |
| | | F: (IR-D) | |
| | | 2.22 | |
| NM_008495 | Mus musculus lectin, galactose binding, | F: (C-IR) | U: 4.6 |
| | soluble 1 (Lgals1), mRNA | 2.65, | (7 to 11) |
| | | U: (C-D) | |
| | | 2.32 | |
| NM_008509 | Mus musculus lipoprotein lipase (Lpl), mRNA | F: (C-D) | F 2.64 |
| | | 2.05, | (5 to 19) |
| | | F: (IR-D) | |
| | | 2.42 | |
| NM_008745 | Mus musculus neurotrophic tyrosine kinase, | U: (C-D) + 2.68 | U: 14.81 |
| | receptor, type 2 (Ntrk2), mRNA | | (Y to O) |
| NM_009127 | Mus musculus stearoyl-Coenzyme A desaturase | F: (C-IR) | U: 2.2 |
| | 1 (Scd1), mRNA | 2.15, | (Y to M) |
| | | F: (C-D) | |
| | | 3.29, | |
| | | F: (IR-D) | |
| | | 2.71 | |
| NM_009255 | Mus musculus serine protease inhibitor 4 | U: (IR-D) | U: 3.6 |
| | (Spi4), mRNA | 2.01 | (5 to 19) |
| | | F: (C-D) | |
| | | 2.61 | |
| NM_009263 | Mus musculus secreted phosphoprotein 1 | F: (C-IR) | F 2.82 |
| | (Spp1), mRNA | 2.04 | (5 to 19) |
| NM_009344 | Mus musculus T-cell death associated gene | U: (IR-D) | F3.29 |
| | (Tdag), mRNA | 2.1 | (7 to 19) |
| | | F: (C-D) | |
| | | 3.91 | |
| NM_009345 | Mus musculus deoxynucleotidyltransferase, | U: (C-D) + 3.66 | U: 2.43 |
| | terminal (Dntt), mRNA | | (Y to O) |
| NM_009669 | Mus musculus amylase 2, pancreatic (Amy2), | F: (C-IR) | F8.34 |
| | mRNA | 3.13 | (5 to 7) |
| | | U: (C-D) | |
| | | 3.23 | |
| NM_009676 | Mus musculus aldehyde oxidase 1 (Aox1), mRNA | F: (C-IR) | U: 2.36 |
| | | 2.08 | (5 to 7) |
| NM_009744 | Mus musculus B-cell leukemia/lymphoma 6 | F: (C-D) | F 2.93 |
| | (Bcl6), mRNA | 4.15, | (5 to 19) |
| | | U: (IR-D) | |
| | | 2.11 | |
| NM_009864 | Mus musculus cadherin 1 (Cdh1), mRNA | F: (C-IR) | F3.24 |
| | | 2.05 | (Y to O) |
| NM_009895 | Mus musculus cytokine inducible | U: (IR-D) | F 2.13 |
| | SH2-containing protein (Cish), mRNA | 2.45 | (Min) |
| | | F: (C-D) | |
| | | 2.25 | |
| NM_009998 | Mus musculus cytochrome P450, 2b10, | F: (C-IR) | U: 2.02 |
| | phenobarbitol inducible, type b (Cyp2b10), | 2.61, | (11to19) |
| | mRNA | F: (C-D) | |
| | | 2.33 | |
| NM_010016 | Mus musculus decay accelerating factor 1 | F: (C-IR) | F 2.11 |
| | (Daf1), mRNA | 2.04, | (7 to 11) |
| | | U: (IR-D) | |
| | | 2.14 | |
| NM_010062 | Mus musculus deoxyribonuclease II alpha | F: (C-IR) | U: 2.89 |
| | (Dnase2a), mRNA | 2.00, | (5 to 11) |
| | | F: (C-D) | |
| | | 2.4 | |
| NM_010107 | Mus musculus ephrin A1 (Efna1), mRNA | F: (C-D) | U: 2.01 |
| | | 2.18 | (5 to 7) |
| NM_010187 | Mus Musculus Fc receptor, IgG, low affinity | F: (C-IR) | F 2.28 |
| | IIb (Fcgr2b), mRNA | 2.18, | (7 to 19) |
| | | U: (IR-D) | |
| | | 2.55 | |
| NM_010225 | Mus musculus forkhead box F2 (Foxf2), mRNA | U: (C-D) + 2.08 | U: 2.42 |
| | | | (5 to 11) |
| NM_010286 | Mus musculus glucocorticoid-induced leucine | U: (C-IR) | F3.32 |
| | zipper (Gilz), mRNA | 2.83, | (5 to 19) |
| | | F: (IR-D) | |
| | | 2.17 | |
| NM_010324 | Mus musculus glutamate oxaloacetate | F: (C-D) | F 2.08 |
| | transaminase 1, soluble (Got1), mRNA | 2.01 | (5 to 11) |
| NM_010354 | Mus musculus gelsolin (Gsn), mRNA | U: (C-IR) | F 2.34 |
| | | 2.03 | (5 to 19) |
| NM_010357 | Mus musculus glutathione S-transferase, | F: (C-IR) | U: 2.11 |
| | alpha 4 (Gsta4), mRNA | 2.17, | (5 to 19) |
| | | F: (C-D) | |
| | | 2.93 | |
| NM_010361 | Mus musculus glutathione S-transferase, | F: (C-IR) | U: 2.14 |
| | theta 2 (Gstt2), mRNA | 2.46, | (5 to 19) |
| | | F: (C-D) | |
| | | 2.25 | |
| NM_010634 | Mus musculus fatty acid binding protein 5, | U: (C-IR) | F 2.84 |
| | epidermal (Fabp5), mRNA | 3.17, | (5 to 19) |
| | | F: (IR-D) | |
| | | 5.62 | |
| NM_011087 | Mus musculus paired-Ig-like receptor A1 | F: (C-D) − 2.49 | F 2.03 |
| | (Piral), mRNA | | (Y to O) |
| NM_011125 | Mus musculus phospholipid transfer protein | F: (C-IR) | U: 3.1 |
| | (Pltp), mRNA | 2.01 | (Y to O) |
| NM_011128 | Mus musculus pancreatic lipase-related | U: (C-D) | U: 2.14 |
| | protein 2 (Pnliprp2), mRNA | 2.35, | (5 to 11) |
| | | U: (IR-D) | |
| | | 2.73 | |
| | | F: (C-D) | |
| | | 2.85 | |
| NM_011146 | Mus musculus peroxisome proliferator | F: (C-IR) | U: 2.68 |
| | activated receptor gamma (Pparg), mRNA | 2.17 | (5 to 11) |
| NM_011375 | Mus musculus sialyltransferase 9 | U: (C-IR) | F 2.12 |
| | (CMP-NeuAc: lactosylceramide | 2.65, | (5 to 19) |
| | alpha-2,3-sialyltransferase) (Siat9), mRNA | U: (C-D) | |
| | | 2.16 | |
| NM_011579 | Mus musculus T-cell specific GTPase (Tgtp), | U: (C-IR) | F 2.1 |
| | mRNA | 2.13 | (5 to 19) |
| | | F: (C-D) | |
| | | 2.1 | |
| NM_011704 | Mus musculus vanin 1 (Vnn1), mRNA | U (C-IR) | U: 2.87 |
| | | 4.37, U | (5 to 7) |
| | | (C-D) | |
| | | 3.14, U | |
| | | (IR-D) | |
| | | 2.37 | |
| NM_012006 | Mus musculus cytosolic acyl-CoA thioesterase | F: (C-D) | U: 3.07 |
| | 1 (Cte1), mRNA | 2.24 | (5 to 7) |
| NM_013459 | Mus musculus adipsin (Adn), mRNA | F: (C-IR) | U: 6.09 |
| | | 2.94 | (5 to 11) |
| NM_013584 | Mus musculus leukemia inhibitory factor | F: (C-IR) | F3.35 |
| | receptor (Lifr), mRNA | 2.31, | (5 to 19) |
| | | F: (C-D) | |
| | | 2.46 | |
| NM_013594 | Mus musculus methyl-CpG binding domain | U: (C-IR) | F 2.35 |
| | protein 1 (Mbd1), mRNA | 2.01, | (5 to 19) |
| | | U: (C-D) | |
| | | 2.15 | |
| NM_013623 | Mus musculus orosomucoid 3 (Orm3), mRNA | U: (C-D) + 4.05 | U: 3.35 |
| | | | (7 to 19) |
| NM_013786 | Mus musculus hydroxysteroid 17-beta | U: (C-D) + 3.68 | F3.08 |
| | dehydrogenase 9 (Hsd17b9), mRNA | | (Y to M) |
| NM_015763 | Mus musculus lipin 1 (Lpin1), mRNA | F: (C-IR) | F4.93 |
| | | 3.7, | (5 to 19) |
| | | U: (C-D) | |
| | | 3.14 | |
| NM_016704 | Mus musculus complement component 6 (C6), | F: (C-IR) | F 2.2 |
| | mRNA | 2.26, | (5 to 19) |
| | | U: (IR-D) | |
| | | 3.29 | |
| NM_016847 | Mus musculus arginine vasopressin receptor | U: (C-IR) | F 2.48 |
| | 1A (Avpr1a), mRNA | 2.02, | (5 to 19) |
| | | F: (IR-D) | |
| | | 2.03 | |
| NM_016875 | Mus musculus Y box protein 2 (Ybx2), mRNA | U: (IR-D) | F 2.26 |
| | | 2.73 | (Y to O) |
| | | F: (C-D) | |
| | | 4.72 | |
| NM_018779 | Mus musculus phosphodiesterase 3A, cGMP | F: (C-IR) | U: 2.15 |
| | inhibited (Pde3a), mRNA | 2.35, | (5 to 19) |
| | | F: (C-D) | |
| | | 2.43 | |
| NM_018861 | Mus musculus solute carrier family 1 | U: (C-IR) | U: 2.25 |
| | (glutamate/neutral amino acid transporter), | 2.18 | (Y to M) |
| | member 4 (Slc1a4), mRNA | | |
| NM_018887 | Mus musculus cytochrome P450, 39a1 | U: (C-D) + 2.54 | F3 |
| | (Oxysterol 7alpha-hydroxylase) | | (7 to 19) |
| | (Cyp39a1-pending), mRNA | | |
| NM_019415 | Mus musculus solute carrier family 12, | U: (C-IR) | U: 2.6 |
| | member 3 (Slcl2a3), mRNA | 2.06 | (5 to 11) |
| NM_019811 | Mus musculus acetyl-Coenzyme A synthetase 1 | F: (C-IR) | U: 2.07 |
| | (AMP forming) (Acas1), mRNA | 2.03, | (Y to M) |
| | | F: (C-D) | |
| | | 2.11 | |
| NM_019922 | Mus musculus cartilage associated protein | U: (C-D) | F 2.03 |
| | (Crtap), mRNA | 2.05 | (11to19) |
| | | F: (C-D) | |
| | | 2.29 | |
| NM_019977 | Mus musculus aldehyde reductase (aldose | U: (C-IR) | U: 2.18 |
| | reductase)-like 6 (Aldrl6), mRNA | 2.51 | (Y to O) |
| | | F: (C-D) | |
| | | 2.15 | |
| NM_019992 | Mus musculus BCR downstream signaling 1 | U: (C-IR) | U: 2.47 |
| | (Brdg1-pending), mRNA | 2.06, | (Y to O) |
| | | U: (C-D) | |
| | | 2.23, | |
| | | U: (IR-D) | |
| | | 2.12 | |
| NM_020277 | Mus musculus long transient receptor | U: (C-D) | U: 3.35 |
| | potential-related channel 5 | 2.05, | (5 to 11) |
| | (Ltrpc5-pending), mRNA | U: (IR-D) | |
| | | 2.32 | |
| | | F: (C-D) | |
| | | 4.69 | |
| NM_020564 | Mus musculus sulfotransferase-related | F: (C-IR) | F 2.32 |
| | protein SULT-X1 (Sult-x1), mRNA | 2.84, | (5 to 19) |
| | | F: (C-D) | |
| | | 2.36, | |
| | | U: (IR-D) | |
| | | 2.6 | |
| NM_020568 | Mus musculus plasma membrane associated | U: (C-D) + 2.12 | U: 6.5 |
| | protein, S3-12 (S3-12-pending), mRNA | | (Y to O) |
| NM_021468 | Mus musculus unc13 homolog (C. elegans) 1 | F: (C-D) − 2.18 | U: 3.58 |
| | (Unc13h1), mRNA | | (M to O) |
| NM_022331 | Mus musculus homocysteine-inducible, | U: (C-IR) | F3.44 |
| | endoplasmic reticulum stress-inducible, | 3.00, | (5 to 19) |
| | ubiquitin-like domain member 1 (Herpud1), | U: (C-D) | |
| | mRNA | 2.29 | |
| NM_023184 | Mus musculus Kruppel-like factor 15 (Klf15), | U: (C-IR) | F 2.87 |
| | mRNA | 2.34 | (5 to 11) |
| NM_023455 | Mus musculus camello-like 4 (Cm14), mRNA | F: (C-IR) | U: 2.75 |
| | | 2.39, | (5 to 19) |
| | | F: (C-D) | |
| | | 2.04 | |
| NM_023740 | Mus musculus RIKEN cDNA 15000151N03 gene | F: (C-IR) | U: 2.04 |
| | (1500015N03Rik), mRNA | 1.7, | (5 to 11) |
| | | F: (C-D) | |
| | | 2.35, | |
| | | U: (IR-D) | |
| | | 2.52 | |
| NM_025404 | Mus musculus RIKEN cDNA 1110036H21 gene | F: (C-IR) | F3.11 |
| | (1110036H21Rik), mRNA | 2.24, | (5 to 11) |
| | | F: (C-D) | |
| | | 2.03 | |
| NM_025429 | Mus musculus serine (or cysteine) proteinase | F: (C-IR) | U: 4.44 |
| | inhibitor, clade B (ovalbumin), member 1a | 3.51, | (5 to 19) |
| | (Serpinbla), mRNA | F: (C-D) | |
| | | 3.01 | |
| NM_026104 | Mus musculus RIKEN cDNA 1700095F04 gene | F: (C-IR) | F 2.72 |
| | (1700095F04Rik), mRNA | 2.22 | (5 to 7) |
| NM_029813 | Mus musculus RIKEN cDNA 2210418O10 gene | F: (C-D) | F 2.28 |
| | (2210418O10Rik), mRNA | 2.4 | (5 to 19) |
| NM_033373 | Mus musculus type I intermediate filament | U: (C-D) + 7.74 | F 2.05 |
| | cytokeratin (Haik1-pending), mRNA | | (Y to O) |
| NM_053215 | Mus musculus RIKEN cDNA 0610033E06 gene | F: (C-IR) | F 2.18 |
| | (0610033E06Rik), mRNA | 1.98, | (5 to 19) |
| | | F: (C-D) | |
| | | 3.23 | |
| U67189 | MMU67189 Mus musculus G protein signaling | U: (C-IR) | U: 2.23 |
| | regulator RGS16 (rgs16) mRNA, complete cds | 3.17 | (Y to M) |
| U70139 | MMU70139 Mus musculus probable nocturnin | U: (C-D) | F 2.05 |
| | protein mRNA, partial cds | 3.08, | (5 to 7) |
| | | U: (IR-D) | |
| | | 2.08 | |
| X03796 | MMALDCR5 Mouse mRNA 5′-region for aldolase C | F: (C-D) − 2.14 | U: 2.61 |
| | (aa 1-227) | | (Y to M) |

TABLE 201
Pairwise Differential Expression Comparisons for
Selected Mouse Genes

| | Age | Age | Age | Age | Age | Age | Age | Age | Age |
|---|---|---|---|---|---|---|---|---|---|
| Gene | 5_7 | 5_11 | 5_19 | 7_11 | 7_19 | 11_19 | Y.M | Y.O | M.O |
| AK002979 | U1.63 | U2.31 | U2.94 | U1.42 | U1.81 | U1.27 | U2.90 | U2.36 | F1.23 |
| AK004387 | F1.79 | F2.93 | F3.29 | F1.64 | F1.84 | F1.12 | F1.40 | F2.33 | F1.67 |
| NM_007702 | U1.22 | F1.07 | U2.59 | U1.30 | U2.13 | U2.78 | U16.09 | U57.01 | U3.54 |
| U67189 | F2.04 | F3.57 | F1.91 | F1.75 | U1.07 | U1.86 | F2.25 | F1.02 | U2.21 |

Differential expression is set forth as the ratio of greater expression level to lesser expression level for the indicated time points. The direction of the change of expression is indicated by “F” (favorable, i.e., younger > older) or “U” (unfavorable, i.e., older > younger). Significant differences (at least two fold) are bold faced.
Note that in identifying a mouse gene as favorable, unfavorable, or mixed, only the significant (at least two fold) differentials are considered.
For the first six comparisons, the time points are weeks, e.g., “7_19” is 7 weeks vs. 19 weeks.
For the last three comparisons, the “Y”, “M” and “O” represent
Y (young) = expression at 118 days
M (medium) = average of expression at 207 and 403 days
O (old) = average of expression at 558 and 725 daus

### Example 2

the Amersham CodeLink™ Uniset Mouse I Bioarray Platform was used (example 1) to identify differences in liver gene expression in aging mice. The mice were fed normal chow and were sacrificed at ages ranging from 35 to 725 days. A total of 190 genes were differentially expressed by at least a 2-fold magnitude (Master Table 1). Analysis of the differentially expressed genes identified CIDE-A as the most differentially expressed gene in liver during this age span. The level of mouse CIDE-A expression in these mice is shown in FIG. 1.

No CIDE-A expression was detected at 35 to 56 days of age (expression level less than 0.2). The expression of CID-A was barely detectable at 118 and 207 days of age (0.36±0.23 and 0.23±0.10, respectivley). However, CIDE-A is readily detected at 403 days of age (3.5±1.99) and the level of expression continues to increase to 7.7 (±0.12) at 558 days of age. Taken together, the level of CIDE-A expression in liver increases at least 38-fold as the mouse progresses from 35 days of age to maximal expression at 558 days of age (7.7±0.12). See FIG. 1.
The differentially expressed gene CIDE-A was subjected to further analysis.
   Northern Analysis

Total RNA (10 ug) from the appropriate tissues was resolved by denaturing agarose gel electrophoresis, transferred to positively charged nylon membrane, hybridized with the [α-³²P]dCTP-labeled mouse CIDE-A cDNA (Random Primed DNA Labeling Kit, Roche, Indianapolis, Ind.) and exposed to Bio-Max MR film (Easman Kodak Co., Rochester, N.Y.).

Immunoblot Analysis

Liver and heart tissue (100 mg) was homogenized in 0.5 ml phosphate buffered saline containing 7.5 ul protease inhibitor cocktail (sigma #P8340, St. Louis, Mo.). The samples were centrifuged for 5 min at 10,000×g. The supernatant was collected and protein concentration determined (Bio-Rad Laboratories #500-0006, Hercules, Calif.). Sixty micrograms of each extract was electrophoresed on a 12.5% SDS-polyacrylamide gel as described previously (25 Bowen). The resolved proteins were transferred to a nitrocellulose membrane and immunoblot-ted using a rabbit anti-mouse CIDE-A polyclonal antibody (*QED Bioscience Inc., San Diego, Calif.) as previously described, se Kelder, B., Richmond, C., Stavnezer, E., List, E. O. and Kopchick, J. J., “Production,characterization and functional activities of v-Ski in cultured cells,” Gene, 202:1521 (1997), and a goat anti-rabbit IgG polyclonal antibody conjugated to horseradish peroxidase.

Liver Histology

Liver tissues fixed in 4% paraformaldehyde were embedded in Tissue Path (Fisher Scientific, Pittsburgh, Pa.). Representative sections were prepared from each liver block, placed on a slide, subjected to H&E staining and evaluated by light microscopy. The percent white 'space was determined as a quantification of the level of steatosis.

Liver Steatosis is Observed in the CXDE-A Expressing Older Mice.

We performed histological examinations on H&E stained liver sections prepared from mice of various ages to determine if increased CIDE-A expression effected any noticeable changes, in the livers of these mice. Among other changes, we noticed an increased level of lipid accumulation within hepatocytes at 725 days of age. There was also an increased level of steatosis in liver tissue isolated from 558 day-old mice but the level of lipid accumulation did not approach that seen at 725 days.

CIDE-A is Expressed at an Early Age in Liver of High-Fat Fed Type-II Diabetic Mice Exhibiting Liver Steatosis.

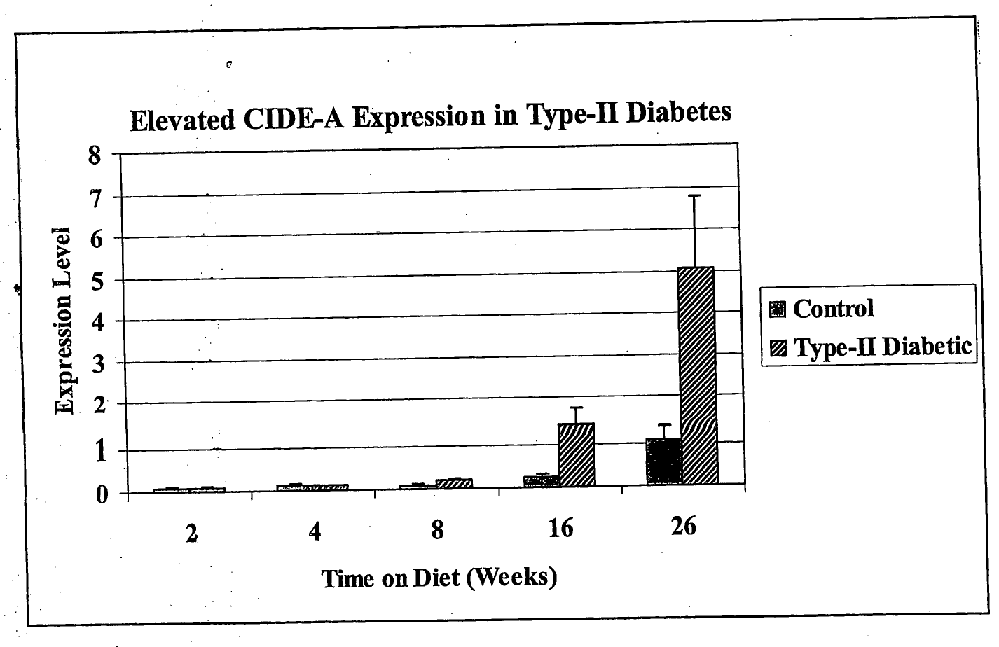

Due to the correlation of increased CIDE-A expression and liver steatosis with increasing age, we investigated whether CIDE-A expression would also be increased in other models of liver steatosis. We utilized a mouse model of diet-induced obesity, hyperinsulinemia and type-II diabetes, see Surwit, R. S., Kuhn, C. M., Cochrane, C., McCubbin, J. A., Feinglos, M. N. (1988) “Diet-induced type-II diabetes in C57BL/6J mice,” Diabetes 37:1163-1167. Mice were weaned onto either a normal diet or a high-fat diet for up to 26 weeks. Representative mice were sacrificed after 2, 4, 8, 16 and 26 weeks on the diet (35, 49, 77, 133 and 203 days of age) and CIDE-A expression levels were determined by DNA microarray analysis (FIG. 2).

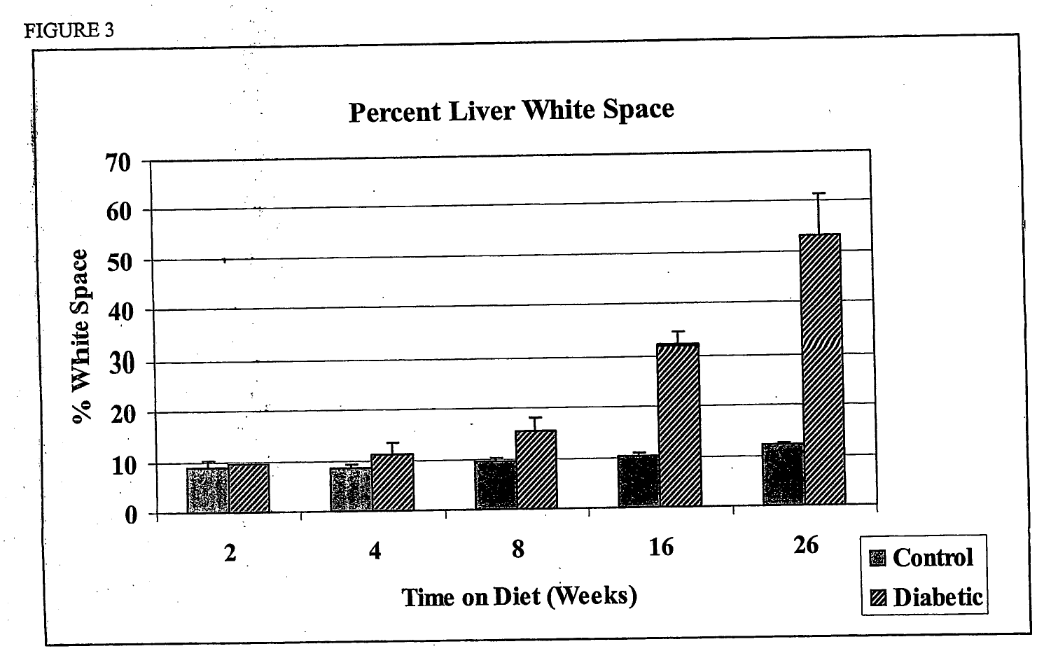

We performed histological examinations on H&E stained liver sections prepared from control and type-II diabetic mice after 2, 16 and 26 weeks of high fat diet feeding (diet started at 3 weeks of age) to assess the degree of diet-induced liver steatosis (FIG. 3). The percent white space of each liver sample was determined by a histomorphometric profiling method using machine vision. H&E stained liver sections isolated from mice fed a normal diet at 56, 558 and 725 days of age shows the accumulation of lipid in liver hepatocytes of older mice.

Histological analysis indicated that diabetic liver hepatocytes accumulate a small amount of lipid as soon as 2 weeks on a high-fat diet and by 8 weeks, liver tissue isolated from high fat-fed mice contain significantly more lipid than their control counterparts. Severe liver steatosis is observed in liver tissues isolated from mice fed the high-fat diet for 16 weeks and is even more pronounced after 26 weeks of high-fat feeding. The percent white space in these livers is 31_{—}6 and 53.2%, respectively. In.comparison, the percent white space in liver tissue of mice fed the normal diet for 16 and 2 6 weeks is 10.3 and 12.2%, respectively. In addition, liver tissue isolated from 16 week high-fat fed hyperinsulinemic mice demonstrate liver steatosis but at a much lower level compared to its diabetic counterpart.

Correlation of CIDE-A Gene Expression and Cell Protein Levels.

Since mRNA levels may not be indicative of the actual level of protein found in the tissue, we performed immunoblot analysis on heart and liver tissue isolated from control, hyperinsulinemic and type-II diabetic mice to confirm the increased CIDE-A levels.

Expression of Genes Involved in Caspase-Dependent Apoptosis

Several groups have reported increase gene expression of members of the caspase-dependent apoptotic pathway such as the FAS death receptor and Fas ligand in hepatocyte steatosis. See Feldstein,supra; Canbay A, Feldstein A E, Higuchi H, Werneburg N, Grambihler A, Bronk S F, Gores G J. (2003) Kupffer cell engulfment of apoptotic bodies stimulates death ligand and cytokine expression. Hepatology 38:1188-1198. We therefore examined the levels of expression of genes involved in this pathway by DNA microarray analysis. A summary of the expression for the genes represented on the microarray is presented in Table 201.

Caspase-3 and -7

Expression levels of Caspase 3 and 7 both decrease from control to hyperinsulinemic to type-II diabetic. But immunohistochemistry on NASH liver sections and a rabbit antibody that recognizes a “neoepitope” (new epitope that is generated upon caspase 3 and 7 cleavage and activation) demonstrated increases in Caspase 3 and 7. activation. The decrease in caspase, 3 and 7 gene expression may be an attempt by the cell to reduce apoptotic signaling within the cell (negative feedback).

Apoptosis in Liver

The level of apoptosis in liver may appear minor. However the rapid phagocytosis of apoptotic bodies-makes the detection of such bodies in tissue extremely difficult, see Savill, J. (2000). Apoptosis in resolutino of inflammation. Kidney Blood Press. Res. 23:173-174. A 4% rate of apoptosis would lead to a 25% reduction in liver tissue in 72 hours, see Schulte-Hermann, R., Bursch, W., Grasl-Kraupp, B. (1995) Active cell death (apoptosis) in liver biology and disease. Prog. Liver Dis. 13:1-35. Therefore, while it may be possible to observe only a small proportion of the ongoing apoptosis, the ongoing cell death may lead to major liver dysfunction.

Alternative Model

While increased apptosis may be a contributing factor to liver dysfunction, we would like to put forth an alternate model for CIDE-A function in liver. In this model: CIDE-A is a part of a redundant apoptotic pathway. According to this model, in the early time points of the genesis of insulin resistance and Type-II diabetes, the liver is capable of managing liver steatosis by the primary caspase-activated apoptotic pathway to eliminate unwanted (lipid accumulating) hepatocytes. However, as the disease progresses (and lipid accumulates), the primary apoptotic pathway becomes overwhelmed (or non-functional) and a secondary (CIDE-A based) pathway is employed as an emergency (last-ditched) effort to maintain liver homeostasis However, this secondary, redundant apoptotic pathway that includes CIDE-A, is either not as efficient or incapable of eliminating the overwhelming lipid accumulation and eventual pathogenesis results.

It is possible that the apoptosis-induced cell death of lipid-containing hepatocytes results in the release of intracellular lipid and the concurrent extracellular liver lipid accumulation. This accumulation may then affect liver functions.

TABLE 201
Expression of genes involved in caspase-dependent apoptosis. The
Amersham CodeLink ™ Uniset Mouse I Bioarray Platform
was used to determine the expression levels of control mice or
high-fat fed mice exhibiting hyperinsulinemia or type-II diabetes after
16 week of feeding (N = 2). Raw expression values are stated, those
resulting in 2-folg ofr greater differential expression are boldfaced.

| | Control | Hyperinsulinemic | Type-II Diabetic |
|---|---|---|---|
| FasL | 0.28 +/− 0.00 | 0.23 +/− 0.01 | 0.14 +/− 0.01 |
| Fas | 2.22 +/− 0.07 | 2.54 +/− 0.08 | 3.31 +/− 0.30 |
| Faim | 1.80 +/− 0.16 | 2.08 +/− 0.13 | 1.61 +/− 0.08 |
| Daxx | 1.19 +/− 0.21 | 1.03 +/− 0.05 | 1.09 +/− 0.11 |
| FADD | 2.86 +/− 0.15 | 3.00 +/− 0.43 | 2.27 +/− 0.12 |
| Caspase-1 | 0.72 +/− 0.03 | 0.93 +/− 0.06 | 0.55 +/− 0.14 |
| Caspase-2 | 1.17 +/− 0.07 | 1.45 +/− 0.09 | 1.24 +/− 0.06 |
| Caspase-3 | 2.03 +/− 0.14 | 1.33 +/− 0.29 | 1.45 +/− 0.43 |
| Caspase-6 | 14.67 +/− 0.71 | 17.41 +/− 2.43 | 18.00 +/− 1.96 |
| Caspase-7 | 4.01 +/− 0.11 | 3.43 +/− 0.66 | 2.89 +/− 0.14 |
| Caspase-8 | 3.81 +/− 0.39 | 3.44 +/− 0.10 | 3.37 +/− 0.63 |
| Caspase-11 | 0.56 +/− 0.00 | 0.73 +/− 0.02 | 0.56 +/− 0.03 |
| Cytochrome C | 49.46 +/− 0.01 | 53.79 +/− 6.79 | 54.59 +/− 4.64 |
| Apaf-1 | 0.26 +/− 0.02 | 0.19 +/− 0.00 | 0.20 +/− 0.03 |
| DFF45 | 1.34 +/− 0.01 | 1.44 +/− 0.13 | 1.76 +/− 0.19 |
| DFF40 | 0.48 +/− 0.04 | 0.55 +/− 0.07 | 0.46 +/− 0.01 |
| Bad | 1.32 +/− 0.06 | 1.29 +/− 0.08 | 1.38 +/− 0.07 |
| Bax | 2.77 +/− 0.29 | 3.58 +/− 0.06 | 3.48 +/− 0.15 |
| Bcl-2L | 0.37 +/− 0.05 | 0.35 +/− 0.08 | 0.45 +/− 0.05 |
| Bcl-2^{a} | 0.29 +/− 0.00 | 0.31 +/− 0.01 | 0.21 +/− 0.03 |
| Ptpn13 | 0.21 +/− 0.00 | 0.15 +/− 0.01 | 0.15 +/− 0.02 |

### REFERENCES

1. Semsei I. (2000) On thae nature of aging. Mech Aging Dev 117:93-108.
2. Sohal, R S, Weindruch, R (1998) Oxidative stress, caloric restriction, and aging. Science 273:59-63.
3. Finch, C E, Revkun, G. (2001) The genetics of aging. Annu. Rev. Genom. Hum. Genet. 2:435-462.
4. Roth, G S, Lasnikov, V, Lesnikov, M, Ingram, D K, Land, M A (2001) Dietary caloric restriction prevents the age-related decline in plasma melatonin levels of rhesus monkeys. J Clin Endocrinol Metab. 86:3292-5.
5. Roth G S, Lane M A, Ingramn D K, Mattison J A, Elahi D, Tobin J D, Muller D, Metter E J (2002) Biomarkers of caloric restriction may predict longevity in humans. Science. 297:811-813.
6. Walford R L, Mock D, Verdery R, MacCallum T. (2002) Calorie restriction in biosphere 2: alterations in physiologic, hematologic, hormonal, and biochemical parameters in humane restricted for a 2-year period. J Gerontol A Biol Sci Med Sci 57:211-24.
7. Kenyon C, Chang J, Gensch E, Rudner A, Tabtiang R. (1993) A C. elegans mutant that lives twice as long as wild type. Nature 366:461-464.
8. Lin, K, Dorman, J B, Rodan, A, Kenyon, C. (1997). daf-16: an HNF-3/Forkhead family member that can function to double the life-span of Caenorhabditis elegans. Science 278, 1319-1322.
9. Clancy D J, Gems D Harshman L G, Oldham S, Stocker H. Hafen
12. Ramaswamy S, Nakamura N, Sansal I, Bergeron L, Sellers W R. (2002) A novel mechanism of gene regulation and tumor suppression by the transcription factor FKHR. Cancer Cell 2002 2:81-91.
13. Hekimi, S, Guarente, L. (2003) Genetics and the specificity of the aging process. Science 299:1351-1354.
14. Brown-Borg, H M, Borg, K E, Meliska, C J, Bartke, A. (1996) Dwarf mice and the aging process. Nature 384:33.
15. Flurkey K, Papaconstantinou J, Miller R A, Harrison D E. (2001) Lifespan extension and delayed immune and collagen aging in mutant mice with defects in growth hormone production. Proc Natl Acad Sci USA 98:6736-6741.
16. Zhou, Y, Xu, B C, Maheshwari, H G, He, L, Reed, M, Lozykowski, M, Okada, S, Cataldo, L, Coschigano, K, Wagner, T E, Baumann, G, Kopchick, J J. (1997) A mammalian model for Laron syndrome produced by targeted disruption of the mouse growth hormone receptor/binding protein gene (the Laron mouse). Proc. Nat. Acad. Sci. USA 94:13215-13220.
17. Coschigano, K, Clemmons, D, Bellush, L L, Kopchick, J J. (2000) Assessment of growth parameters and life-span of GHR/BP gene-disrupted mice. Endocrinology 141:2 608-2613.
17a. Coschigano, K T, Holland, A N, Riders, M E, List, E O, Flyvberg, A, Kopchick, J J, Deletion, but not antagonism, of the mouse growth hormone receptor results in severely decreased body weights, insulin and IGF-1 levels and increased lifespan, Endocrinology (electronically published May 30, 2003 as doi:10.1210/en.2003-0374)
18. Iolzenberger M, Dupont J, Ducos B, Leneuve P, Geloen A, Even P C, Cervera P, Le Bouc Y. (2003) IGF-1 receptor regulates lifespan an-d resistance to oxidative stress in mice. Nature 421:182-187.
19. Migliaccio E, Giorgio M, Mele S, Pelicci G, Reboldi P, Pandolfi P P, Lanfrancone L, Pelicci P G. (1999) The p66shc adaptor protein controls oxidative stress response and life span in mammals. Nature 402:309-313.
20. Bartke A, Wright J C, Mattison J A, Ingram D K, Miller R A, Roth G S. (2001) Extending the lifespan of long-lived mice. Nature 414:412.
21. Weindruch R, Kayo T, Lee C K, Prolla T A. (2002) Gene expression profiling of aging using DNA microarrays. Mech Aging Dev 123:177-193.
22. Lee C K, Allison D B, Brand J, Weindruch R, Prolla T A. (2002) Transcriptional profiles associated with aging and middle age-onset caloric restriction in mouse hearts. Proc Natl Acad Sci USA 99:14988-14993.
23. Prolla T A. (2002) DNA microarray analysis of the aging brain. Chem Senses 27299-306.

Citation of documents herein is riot intended as an admission that any of the documents cited herein is pertinent prior art, or an admission that the cited documents is considered material to the patentability of any of the claims of the present application. All statements as to the date or representation as to the contents of these documents is based on the information available to the applicant and does not constitute any admission as to the correctness of the dates or contents of these documents.

The appended claims are to be treated as a non-limiting recitation of preferred embodiments.

In addition to those set forth elsewhere, the following references are hereby incorporated by reference, in their most recent editions as of the time of filing of this application: Kay, Phage Display of Peptides and Proteins: A Laboratory Manual; the John Wiley and Sons Current Protocols series, including Ausubel, Current Protocols in Molecular Biology; Coligan, Current Protocols in Protein Science; Coligan, Current Protocols in Immunology; Current Protocols in Human Genetics; Current Protocols in Cytometry; Current Protocols in Pharmacology; Current Protocols in Neuroscience; Current Protocols in Cell Biology; Current Protocols in Toxicology; Current Protocols in Field Analytical Chemistry; Current Protocols in Nucleic Acid Chemistry; and Current Protocols in Human Genetics; and the following Cold Spring Harbor Laboratory publications: Sambrook, Molecular Cloning: A Laboratory Manual; Harlow, Antibodies: A Laboratory Manual; Manipulating the Mouse Embryo: A Laboratory Manual; Methods in Yeast Genetics: A Cold Spring Harbor Laboratory Course Manual; Drosophila Protocols; Imaging Neurons: A Laboratory Manual; Early Development of Xenopus laevis: A Laboratory Manual; Using Antibodies: A Laboratory Manual; At the Bench: A Laboratory Navigator; Cells: A Laboratory Manual; Methods in Yeast Genetics: A Laboratory Course Manual; Discovering Neurons: The Experimental Basis of Neuroscience; Genome Analysis: A Laboratory Manual Series; Laboratory DNA Science; Strategies for Protein Purification and Characterization: A Laboratory Course Manual; Genetic Analysis of Pathogenic Bacteria: A Laboratory Manual; PCR Primer: A Laboratory Manual; Methods in Plant Molecular Biology: A Laboratory Course Manual; Manipulating the Mouse Embryo: A Laboratory Manual; Molecular Probes of the Nervous System; Experiments with Fission Yeast: A Laboratory Course Manual; A Short Course in Bacterial Genetics: A Laboratory Manual and Handbook for Escherichia coli and Related Bacteria; DNA Science: A First Course in Recombinant DNA Technology; Methods in Yeast Genetics: A Laboratory Course Manual; Molecular Biology of Plants: A Laboratory Course Manual.

All references cited herein, including journal articles or abstracts, published, corresponding, prior or otherwise related U.S. or foreign patent applications, issued U.S. or foreign patents, or any other references, are entirely incorporated by reference herein, including all data, tables, figures,: and text presented in the cited references. Additionally, the entire contents of the references cited within the references cited herein are also entirely incorporated by reference.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

Any description of a class or range as being useful or preferred in the practice of the invention shall he deemed a description of any subclass (e.g., a disclosed class with one or more disclosed members omitted) or subrange contained therein, as well as a separate description of each individual member or value in said class or range.

The description of preferred embodiments individually shall be deemed a description of any possible combination of such preferred embodiments, except for combinations which are impossible (e.g, mutually exclusive choices for an element of the invention) or which are expressly excluded by this specification.

If an embodiment of this invention is disclosed in the prior art, the description of the invention shall be deemed to include the invention as herein disclosed with such embodiment excised.

LENGTHY TABLE
The patent application contains a lengthy table section. A copy of the table is available in electronic form from the USPTO web site (http://seqdata.uspto.gov/?pageRequest=docDetail&DocID=US20070111933A1). An electronic copy of the table will also be available from the USPTO upon request and payment of the fee set forth in 37 CFR 1.19(b)(3).

1. A method of (I) reducing a rate of biological aging in a human subject, and/or (II) delaying the time of onset, or reducing the severity, of an undesirable age-related phenotype, and/or (III) protecting against an age-related (senescent) disease, which comprises administering to the subject a protective amount of an agent which is
(1) a polypeptide which is substantially structurally identical or conservatively identical in sequence to a reference protein which is (a) selected from the group consisting of mouse and human proteins set forth in master table 1, subtable 1A, or (b) selected from the group consisting of human proteins within at least one of the human protein classes set forth in master table 2, subtable 2A,
(2) an expression vector encoding the polypeptide of (1) above and expressible in a human cell, under conditions conducive to expression of the polypeptide of (1);
(3) an antagonist of a Polypeptide, occurring in said subject, which is substantially structurally identical or conservatively identical in sequence to a reference protein which is (a) selected from the group consisting of mouse and human proteins set forth in master table 1, subtable 1B, or (b) selected from the group consisting of human proteins belonging to at least one of the human protein classes set forth in master table 2, subtable 2B, or
(4) an anti-sense vector which inhibits expression, in said subject, of a polypeptide, occurring in said subject, which is substantially structurally identical or conservatively identical in sequence to a reference protein which is (a) selected from the group consisting of mouse and human proteins set forth in master table 1, subtable 1B, or (b) selected from the group consisting of human proteins belonging to at least one of the human protein classes set forth in master table 2, subtable 2B,
where said agent reduces a rate of biological aging in said subject, and/or delays the time of onset, or reduces the severity, of an undesirable age-related phenotype in said subject, and/or protects against an age-related disease.

2. (canceled)

3. A method of determining a biological age of a human subject, or a rate of biological aging of a human subject, which comprises
1) assaying tissue or body fluid samples from said subjects to determine the level of expression of a “favorable” human marker gene, said human marker gene encoding a human protein which is substantially structurally identical or conservatively identical in sequence to a reference protein which is (a) selected from the group consisting of mouse and human proteins set forth in master table 1, subtable 1A, or (b) selected from the group consisting of human proteins within at least one of the human protein classes set forth in master table 2, subtable 2A, and inversely correlating the level of expression of said marker gene with a biological age or a rate of biological aging of said patient, or
2) assaying tissue or body fluid samples from said subjects to determine the level of expression of an “unfavorable” human marker gene, said human marker gene encoding a human protein which is substantially structurally identical or conservatively identical in sequence to a reference protein which is (a) selected from the group consisting of mouse and human proteins set forth in master table 1, subtable 1B, or (b) selected from the group consisting of human proteins belonging to at least one of the human protein classes set forth in master table 2, subtable 2B,
and directly correlating the level of expression of said marker gene with a biological age or a rate of biological aging of said subject.

4. (canceled)

5. The method of claim 1 in which (I) applies.

6-7. (canceled)

8. The method of claim 5 in which biological age is measured by a biomarker.

9. The method of claim 8 in which the marker is a simple biomarker.

10. The method of claim 8 in which the marker is a composite biomarker.

11. The method of claim 5 in which the affected biological age is the overall biological age of the subject.

12. The method of claim 5 in which the affected biological age is the biological age of a body system of the subject.

13. The method of claim 5 in which the affected biological age is the biological age of an organ of the subject.

14. The method of claim 13 in which the organ is the liver.

15. The method of claim 8 in which at least one marker is the level of a biochemical in the blood of the subject.

16. The method of claim 15 in which the biochemical is growth hormone or IGF-1.

17. The method of claim 1 in which (a) applies.

18. The method of claim 1 in which the reference protein is a human protein.

19. The method of claim 1 in which the reference protein is a mouse protein.

20. The method of claim 3 in which the level of expression of the marker protein is ascertained by measuring the level of the corresponding messenger RNA.

21. The method of claim 3 in which the level of expression is ascertained by measuring the level of a protein encoded by said marker gene.

22. The method of claim 1 in which said polypeptide is at least 80% identical or at least highly conservatively identical to said reference protein.

23. The method of claim 1 in which said polypeptide is at least 90% identical to said reference protein.

24. The method of claim 23 in which said polypeptide is identical to said reference protein.

25-27. (canceled)

28. The method of claim claim 35, in which the antagonist is an antibody, or an antigen-specific binding fragment of an antibody.

29. The method of claim claim 35, in which the antagonist is a peptide, peptoid, nucleic acid, or peptide nucleic acid oligomer.

30. The method of claim claim 35, in which the antagonist is an organic molecule with a molecular weight of less than 500 daltons.

31. The method of claim 30 in which said organic molecule is identifiable as a molecule which binds said polypeptide by screening a combinatorial library.

32. The method of claim 35, in which the marker protein is CIDE-A.

33. The method of claim 1 in which the agent is the agent of (1) or (2).

34. The method of claim 1 in which the agent is the agent of (1).

35. The method of claim 1 in which the agent is the agent of (3) or (4).

36. The method of claim 1 in which the agent is the agent of (3).

37. The method of claim 3 in which (1) applies.

38. The method of claim 3 in which (2) applies.
